(19) United States
(12) Patent Application Publication
(10) Pub. No.: US 2021/0337826 A1
(43) Pub. Date: Nov. 4, 2021

(54) MODIFICATION OF PROTEIN GLYCOSYLATION IN MICROORGANISMS

(71) Applicant: Clara Foods Co., South San Francisco, CA (US)

(72) Inventors: Frank Douglas Ivey, South San Francisco, CA (US); Joel Andrew Kreps, South San Francisco, CA (US); Jason Helvey, South San Francisco, CA (US); David Anchel, South San Francisco, CA (US)

(21) Appl. No.: 17/179,100

(22) Filed: Feb. 18, 2021

Related U.S. Application Data
(63) Continuation of application No. PCT/US19/47521, filed on Aug. 21, 2019.
(60) Provisional application No. 62/720,785, filed on Aug. 21, 2018.

(51) Int. Cl. A23J 3/04; C12N 9/24; C12N 15/81; A23L 33/17; A23L 2/66

(52) U.S. Cl. CPC A23J 3/04; C12N 9/2402; C12N 15/815; A23V 2002/00; A23L 33/17; A23L 2/66; C12Y 302/0113

(57) ABSTRACT

The present disclosure contemplates methods for modifying post-translational modification of proteins recombinantly expressed a microbial host to improve one or more properties of the recombinant protein.

### CROSS-REFERENCE

This application is a Continuation application of International Patent Application PCT/US2019/047521 (Attorney Docket No. 49160-712.601), filed Aug. 21, 2019, which claims the benefit of U.S. Provisional Patent Application No. 62/720,785 (Attorney Docket No. 49160-712.101), filed Aug. 21, 2018; each of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Sep. 20, 2019, is named 49160 712 601 SL.txt and is 262,767 bytes in size.

### BACKGROUND OF THE INVENTION

There is a need to identify methods for creating proteins, especially for human and animal consumption, to provide enhanced safety, efficacy and nutritional value. Protein production in microbial hosts can be a valuable tool for protein production. However, post translational modifications (PTMs) of a recombinant protein peptide backbone can affect enzymatic efficacy, safety, ease of purification, secretion, and/or expression level of the protein.

For example, heterologous proteins produced in Pichia pastoris have been known to be “hypermannosylated”, in that the glycosylation sites of their peptide backbone can carry extended branches of mannosyl groups (sometimes exceeding 100 mannose groups; Ser Huy Teh,¹ Mun Yik Fong,² and Zulqarnain Mohamed^{1,3} Genet Mol Biol. 2011 July-September; 34(3): 464-470.). Such aberrant glycosylation can raise the risk of immunogenicity in cases where the heterologous protein is intended for therapeutic use.

In some cases, PTMs can be beneficial to the recombinant protein's intended use, however, there are instances in which a host's PTMs confers unwanted covalent attachments that are detrimental. There is a need to identify methods for creating proteins, especially for human and animal consumption, with improved methods to express a desired PTM profile to take advantage of the beneficial aspects of PTMs while avoiding detrimental characteristics.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

Provided herein are methods, protein sequences and products for producing animal proteins in a microbial host which incorporate advantageous PTMs and avoid other unwanted effects of PTMs. In some embodiments, the methods, components and resulting products herein utilize modifications of PTMs to improve the nutritional content and/or nutritional value of recombinant animal proteins produced in a microbial host. In some embodiments, the nutritional content and/or nutritional value is improved by altering the glycosylation of the recombinant protein produced by the microbial host.

In some embodiments, the recombinant protein finds use in food, nutritional or other products for human or animal consumption. In some embodiments, the recombinant protein may be an enzyme for use in one or more industrial processes.

Provided herein are methods of producing a consumable composition. The methods may comprise recombinantly expressing a nutritional protein in a host cell. wherein the nutritional protein may be secreted out of the host cell. The method may also comprise recombinantly expressing an α-1,2-mannosidase in the host cell. The α-1,2-mannosidase may reduce the glycosylation of greater than 50% of the nutritional protein secreted from the host cell. The nutritional protein may be mixed with at least one more component to form the consumable composition.

The α-1,2-mannosidase may have a sequence of SEQ ID No: 7, a functional equivalent thereof or a sequence homology of 85% or more identical to SEQ ID No: 7. The α-1,2-mannosidase may have a sequence of SEQ ID No: 150, a functional equivalent thereof or a sequence homology of 85% or more identical to SEQ ID No: 150.

The nutritional content of the consumable composition may be equal to or greater than the nutritional content of a control composition wherein the control composition is produced using the same protein isolated from a native source or the recombinant nutritional protein un-modified by the α-1,2-mannosidase.

The nutritional content may be a protein content of the composition. The protein content of the consumable composition may be at least 5% higher than the control composition. The protein content of the consumable composition may be at least 10% higher than the control composition. The protein content of the consumable composition may be at least 20% higher than the control composition.

At least 50% of the nutritional protein secreted from the host cell may have a modified glycosylation pattern. At least 75% of the nutritional protein secreted from the host cell may have a modified glycosylation pattern. At least 80% of the nutritional protein secreted from the host cell may have a modified glycosylation pattern. At least 90% of the nutritional protein secreted from the host cell may have a modified glycosylation pattern.

The thermal stability of the nutritional protein having a modified glycosylation pattern may be increased as compared to a control composition wherein the control composition is produced using the same protein isolated from a native source or the recombinant nutritional protein un-modified by the α-1,2-mannosidase.

The host cell may be a Pichia species, such as Pichia pastoris.

The nitrogen to carbon ratio of the nutritional protein may be equal to or greater than the ratio of the nutritional protein isolated from its native source.

The nutritional protein may be an animal protein. The nutritional protein may be an avian protein. The nutritional protein may be an egg-white protein.

In some embodiments, a consumable composition may be produced using the methods described herein. The consumable composition may be a beverage. The consumable composition may be a foodstuff.

In some embodiments, provided herein is a host cell used for the expression of a recombinant nutritional protein. The host cell may comprise a first promoter driving expression of a nutritional protein and a second promoter driving expression of an α-1,2-mannosidase with sequence of SEQ ID Nos: 7 or 150, a functional equivalent thereof or a sequence 85% or more identical to SEQ ID Nos: 7 or 150. The mannosylation of the nutritive protein may be reduced as a result of the expression of the α-1,2-mannosidase. The host cell may be a fungus or a yeast. The host cell may be a Pichia species, such as Pichia pastoris.

The nutritional protein and the α-1,2-mannosidase may be expressed using one or more expression cassettes. The nutritional protein and the α-1,2-mannosidase may be expressed on separate expression constructs.

The nutritional protein may be secreted out of the host cell. The secreted nutritive protein may have an equal to or higher nutritive content as compared to a control composition wherein the control composition is produced using the same protein isolated from a native source or the recombinant nutritional protein un-modified by the α-1,2-mannosidase.

The nutritive content may be the protein content. The secreted nutritive protein may have varying degrees of glycosylation. At least 50% of the secreted nutritive protein may have a modified glycosylation pattern.

Provided herein are consumable compositions. The consumable composition may comprise a recombinant animal protein produced in a heterologous host cell and one or more additional ingredients. The animal protein may comprise a level of glycosylation suitable for use in a consumable composition. The animal protein may provide one or more food-functional features to the consumable composition.

In some embodiments, provided herein are microorganisms comprising a first nucleic acid encoding a nutritive protein and a second nucleic acid encoding an α-1,2-mannosidase. The α-1,2-mannosidase may be heterologous to the microorganism and the α-1,2-mannosidase may be capable of modifying the glycosylation structure of the nutritive protein.

The nutritive protein may be used as a food ingredient or food product. The α-1,2-mannosidase may comprise an amino acid sequence of SEQ ID NO:150, SEQ ID NO:7 or a sequence with greater than 80% or 85% homology thereto.

The first and second nucleic acid sequences may be contained in one or more expression cassettes. The microorganism may be a Pichia species. The α-1,2 mannosidase may be a Gallus gallus α-1,2 mannosidase. The α-1,2 mannosidase may be a Trichoderma reesei α-1,2 mannosidase and the microorganism may be a Pichia species.

The nutritive protein may be an egg white protein. The egg white protein may comprise an amino acid sequence of any one of SEQ ID Nos: 11-26 or any sequence having 80% homology thereto. At least one of the nucleic acid sequences may be codon optimized for expression in the microorganism.

In some embodiments, the recombinant animal protein expressed in the microbial host has nutritional value and can be used on its own or in compositions as a source of nutrition. In some embodiments, the heterologously expressed protein is a nutritional source of protein for an animal or human. In some embodiments herein, the modification of glycosylation of a recombinant animal protein alters the ratio of nitrogen to carbon in the protein as compared to the same recombinant protein expressed in the microbial host cell without modification of its glycosylation structure. In some embodiments, the modification of glycosylation alters or increases the nutritional value of the recombinant animal protein in comparison to the protein from its naturally occurring source.

In some embodiments, the recombinant animal protein has enzymatic activity. In some embodiments, the recombinant animal protein has functionality for use in industrial processes. In some embodiments, the modification of glycosylation of the recombinant animal protein enhances, reduces or otherwise alters one or more functional properties of the recombinant protein as compared to the same protein expressed without modification of its glycosylation structure.

In some embodiments of the methods herein, the steps include altering the glycosylation machinery of the microbial host by altering, deleting or adding one or more glycosylation enzymes. In some embodiments, the alteration of the microbial host's glycosylation machinery results in the production of a recombinant protein with improved nutritional content or improved nutritional value. In some embodiments, the microbial host for use in the methods is a filamentous fungi. In some embodiments, the microbial host is Pichia pastoris (now known as Komagataella phaffii).

In some embodiments herein, the nutritional content or nutritional value of the recombinantly expressed animal protein is improved by also expressing an alpha-1,2 mannosidase (α-1,2 mannosidase) in the microbial host. In some embodiments of the method, the steps include recombinantly expressing an animal protein in a filamentous fungi host cell; recombinantly expressing an alpha-1,2 mannosidase (α-1,2 mannosidase) in the same host cell; and isolating the recombinant animal protein from the host. In some embodiments of the method, the microorganism for recombinant expression is altered in two or more components of the glycosylation machinery. Such alterations can include, for example, a deletion or knockout of OCH1 in a yeast host.

In some embodiments of the method, the recombinant animal protein is secreted from the host cell, and the α-1,2 mannosidase is not secreted from the host cell. In some embodiments of the method, the α-1,2 mannosidase is expressed without any heterologous secretion signal or heterologous intra-cellular targeting sequence and the recombinant animal protein is expressed with a secretion signal sequence or other amino acid sequence that results in the secretion of the animal protein. In this case the α-1,2 mannosidase is retained inside the cell because the host recognizes a non-native localization signal, the α-1,2 mannosidase acts on the recombinantly expressed animal protein inside the cell and then the recombinant animal protein with the altered glycosylation modification is secreted. In some embodiments of the method, the secreted animal protein may then be isolated apart from the mannosidase and other microbial-related proteins. In some embodiments of the method, the recombinant animal protein is isolated from growth medium external to the host cell.

In some embodiments of the method, the α-1,2 mannosidase is heterologous to the microbial host cell. The α-1,2 mannosidase may be from a fungal source, an avian source, or a mammalian source. In some embodiments, the α-1,2 mannosidase is derived from Trichoderma reesei. In other embodiments, the α-1,2 mannosidase is derived from an avian species such as the species Gallus gallus. In some embodiments, two or more α-1,2 mannosidase proteins are recombinantly expressed in the method. The two or more α-1,2 mannosidase proteins may be derived from the same, similar or different species. In some embodiments, the one or more α-1,2 mannosidase proteins for expression is any one or more of SEQ ID: Nos. 1-10, or 145-151, an amino acid sequence encoded by SEQ ID Nos. 152-153, or a sequence having at least 80% or 85% homology thereto.

In some embodiments, the one or more α-1,2 mannosidases are expressed in a host cell that also recombinantly expressed a recombinant animal protein. In some embodiments, the microorganism contains the first and second nucleic acid sequences that are contained in one or more expression cassettes. These cassettes may be integrated at one or more sites in the host genome through homologous or non-homologous recombination. In some embodiments, the first and second nucleic acid sequences are contained in the same expression cassette. In other embodiments, the first and second nucleic acid sequences are contained in separate expression cassettes, and these separate cassettes may be integrated into the host genome together, separately, concomitantly or sequentially.

In some embodiments, the first nucleic acid further contains a heterologous promoter. In some embodiments, the second nucleic acid contains a heterologous promoter. In some embodiments, the first and second nucleic acids may each contain a heterologous promoter, and such promoters may be the same or different from one another.

The methods herein for expressing α-1,2 mannosidase and a recombinant animal protein include a variety of host microorganisms including yeasts. In some embodiments of the methods, the microorganism is a methylotrophic yeast. In some embodiments, the yeast is a Pichia sp. or a Komagataella sp. In some embodiments, the yeast is Pichia Pastoris or Komagataella phaffii.

The methods provided herein are amenable to the production of a recombinant animal protein with improved nutritional content or improved nutritional value. In some embodiments, the improved nutritional content or improved nutritional value alters the nitrogen to carbon ratio of recombinant animal protein. In some embodiments the nitrogen to carbon ratio of recombinant animal protein is greater than about 0.25, about 0.3, about 0.35 and/or about 0.4. In some embodiments, the recombinant animal protein has a degree of glycosylation that is equal to or reduced as compared with the animal protein when isolated from its naturally-occurring source.

In some embodiments, the recombinant animal protein is equal to or reduced in mannosylation as compared with the protein when isolated from its naturally-occurring source. In some embodiments, the recombinantly produced animal protein contains one or more Man₅GlcNAc₂ residues. In some embodiments, the recombinant animal protein has a proportion of Man₅GlcNAc₂ that is greater than the proportion of Man₈GlcNAc₂ associated with the protein. In some embodiments, the recombinant animal protein has a ratio of ManₓGlcNAc₂ to Man_{y}GlcNAc₂ is greater than 1, and X of ManₓGlcNAc₂s an integer selected from 1, 2, 3, 4, and 5, and Y of Man_{y}GlcNAc₂ is an integer greater than or equal to 6. In some embodiments, Y is an integer selected from 6, 7, 8, 9 and 10. Provided herein are compositions containing one or more recombinant animal protein(s), having one or more Man₅GlcNAc₂ residues where the recombinant protein has an improved nutritional content or improved nutritional value. In some embodiments, the improved nutritional content or improved nutritional value includes having a nitrogen to carbon ratio of the recombinant animal protein that is greater than or equal to about 0.25, about 0.30, about 0.35, or about 0.4.

The compositions described herein can be formulated as a foodstuff, a nutritional supplement, a nutritional powder, or a consumable drink. The compositions described herein can also be formulated as an animal feed or feed supplement.

In some embodiments of the methods and compositions herein, the recombinant animal protein is a recombinant egg white protein. In some embodiments, the egg white protein is one or more of ovomucoid (OVD), ovalbumin (OVA), ovoglobulin, β-ovomucin, α-ovomucin and lysozyme. In some embodiments, the recombinant animal protein is a recombinant egg white protein and the host cell for protein production is Pichia. In some embodiments, the recombinant animal protein is a recombinant egg white protein and the glycosylation structure of the expressed protein in Pichia is modified such that the ratio of nitrogen to carbon of the recombinant egg white protein is equal to or greater than the egg white protein when isolated from naturally-occurring chicken egg. In some embodiments, the recombinant animal protein is a recombinant egg white protein and the glycosylation structure of the expressed protein in Pichia is modified such that the nutritional value of the protein is substantially the same as or better than the protein from its native source.

In some embodiments, the recombinant egg white protein has a degree of glycosylation that is equal to or reduced as compared with the egg white protein when isolated from naturally-occurring chicken egg. In some embodiments, the recombinant egg white protein is equal to or reduced in mannosylation as compared with the egg white protein when isolated from naturally-occurring chicken egg. In some embodiments, the recombinant egg white protein contains one or more Man₅GlcNAc₂ residues. In some embodiments, the recombinant egg white protein has a proportion of Man₅GlcNAc₂ that is greater than the proportion of Man₈GlcNAc₂ associated with the egg white protein. In some embodiments, the recombinant egg white protein has a ratio of ManₓGlcNAc₂ to Man_{y}GlcNAc₂ is greater than 1, and X of ManₓGlcNAc₂s an integer selected from 1, 2, 3, 4, and 5, and Y of Man_{y}GlcNAc₂ is an integer greater than or equal to 6. In some embodiments, Y is an integer selected from 6, 7, 8, 9 and 10.

The methods provided herein are amenable to the production of a recombinant egg white protein such that the nitrogen to carbon ratio of recombinant egg white protein is greater than about 0.25, about 0.3, about 0.35 and/or about 0.4. In some embodiments, the composition contains a second egg white protein which may be a native egg white protein, a recombinant egg white protein or an egg white protein (native or recombinant) that has been modified to alter the glycosylation structure and/or nitrogen to carbon ratio of the second protein. The compositions produced by the methods described herein can be formulated as a foodstuff, a nutritional supplement, a nutritional powder, or a consumable drink.

In some embodiments, the recombinant egg white protein with the altered nitrogen to carbon ratio is ovomucoid, ovalbumin, ovoglobulin, β-ovomucin, α-ovomucin, cystatin, ovoinhibitor and lysozyme. In some embodiments, the recombinant egg white protein according with the altered nitrogen to carbon ratio is any one or more of proteins set forth in SEQ ID NOs: 11-26 or a sequence having at least 80% homology thereto.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1D illustrate ManₓGlcNAc₂ substructures.

FIG. 2 illustrates an exemplary vector comprising a promoter operably linked to a transgene.

FIGS. 3A-B illustrate mass spectra results for samples showing the relative amounts of each glycoform present in samples.

FIGS. 4A-B illustrate SDS-Page band patterning of Strain 2 (a TrMDS2 expressing strain) compared to its parent strain Strain 1 in SF17 (a) and SF22 (b). The 2 strains produce a similar amount of OVD. Strain 1 produces the characteristic OVD pattern seen in K. phaffii thus far with 7 main bands labeled in (a). With the exception of bands 6 and 7, all the main bands appear to have shifted.

FIG. 5 illustrates Common N-glycosylation patterns of K. phaffii. A square indicates N-acetylglucosamine (GlcNAc) while circles indicate mannose (Man).

FIG. 6 illustrates a comparison of deglycosylation function of TrMDS2 and GgMAN1A1.

FIG. 7 illustrates a result of coexpression of TrMDS2 and GgMAN1A1.

FIG. 8 illustrates SDS-PAGE results of culture supernatants of individual transformants expressing HsORM1.

FIGS. 9A-C illustrate SDS-PAGE results of TrMDS2-induced deglycosylation of HsORM1 and the vector schematic used for transformation.

FIG. 10 illustrates SDS-PAGE results of the deglycosylation of Ovalbumin (OVA).

FIG. 11 illustrates SDS-PAGE results of native OVA and denatured OVA.

FIG. 12 illustrates SDS-PAGE results of the deglycosylation of OVA with TrMDS2.

FIG. 13 illustrates results of lack of deglycosylation activity of MDS1 on GgOVD.

FIG. 14 illustrates results of the deglycosylation activity of TrMDS2 on GgOVD.

### DETAILED DESCRIPTION OF THE INVENTION

The methods, nucleic acids, expression constructs, microorganisms, compositions and methods provided herein provide tools, methods and compositions for expressing recombinant animal protein in a host and modifying the glycosylation of the expressed protein. One such host contemplated herein is Pichia sp. (now reclassified as Komagataella sp.) The present disclosure contemplates modifying a Pichia species glycosylation machinery, such as in a Pichia pastoris in any one or more of the methods described herein.

The present disclosure contemplates modifying glycosylation of the recombinant protein to alter or enhance one or more functional characteristics of the protein and/or its production.

By such modifications, a recombinant protein can be made that has a higher nutrition value as compared to the recombinant protein produced in the host microorganism absent modification to the glycosylation machinery. The recombinant animal protein may have a higher nitrogen to carbon ratio as compared to the recombinant protein produced in the host microorganism absent modification to the glycosylation machinery, and/or as compared to the same protein produced from its native source or another heterologous host. By such modifications, in concert with recombinantly expressing one or more proteins, a recombinant protein can be made that has improved expression, secretion, purification as compared to the recombinant protein produced in the host absent modification to the glycosylation machinery. By such modifications, in concert with recombinantly expressing one or more proteins, a recombinant protein can be made that has improved enzymatic functionality or activity as compared to the recombinant protein produced in the host microorganism absent modification to the glycosylation machinery.

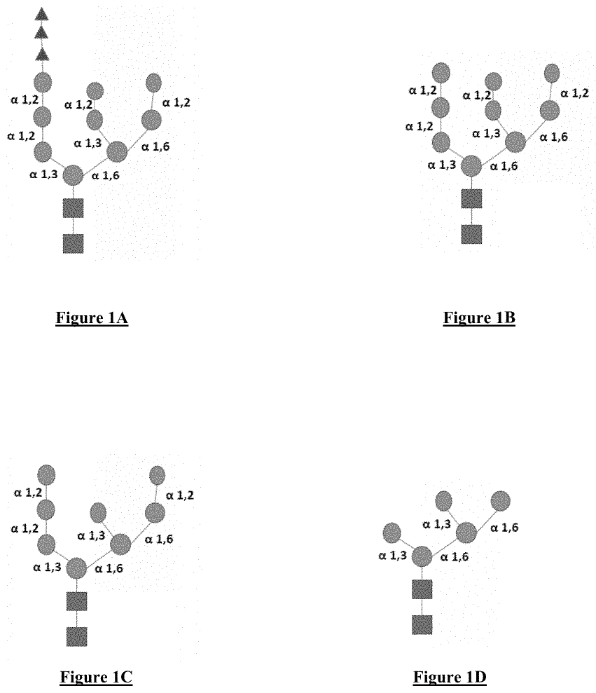

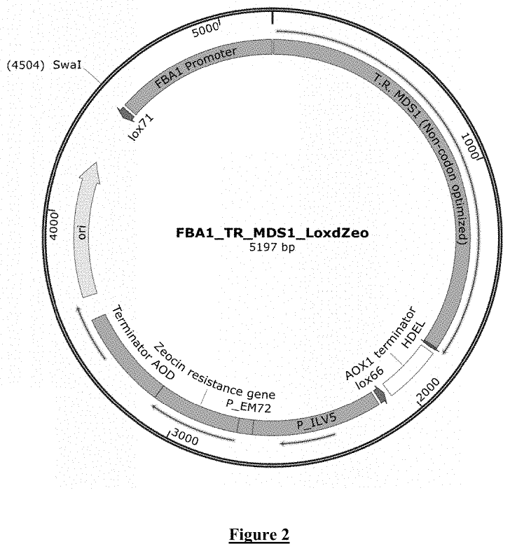

One approach to effect glycosylation in a yeast host exploits the required alpha-1,6-Mannosyltransferase activity of OCH1 protein in the Golgi on the core Man₈GlcNAc₂ substrate (FIG. 1C) as a necessary step for further extending mannosylation of the glycan structure in what is deemed “outer chain elongation”. In knockouts or mutants with disrupted OCH1 function, mannosylation cannot proceed past this base substrate in the Golgi, and hypermannosylation is eliminated.

In some embodiments, the yeast host may be modified to knockout OCH1 function. In some embodiments, the yeast host may be modified to have a partial disruption or knockdown of OCH1 function.

Alternatively, or additionally, one can also knock in an ER resident, heterologous mannosidase such as Trichoderma reesei alpha-1,2 mannosidase, or other similarly functional enzymes, to cleave glycans to Man₅GlcNAc₂ core structures before a nascent polypeptide's translocation to the Golgi, thereby effectively eliminating the Man₈GlcNAc₂ substrate required for efficient alpha-1,6-Mannosyltransferase activity of OCH1. It has been suggested that OCH1's alpha-1,6-Mannosyltransferase activity is specific for the Man₈GlcNAc₂ glycan structure and not the Man₅GlcNAc₂ structure. It is therefore possible that OCH1 activity can be effectively eliminated if the majority of peptide bound ER-processed glycan structures translocated to the Golgi are cleaved to Man₅GlcNAc₂ structures by the activity of an ER resident, heterologous alpha-1,2-mannosidase. Following this rationale, disclosed here in a simplified method of making a microorganism with altered glycosylation relative to wild type, wherein the microorganism only comprises one or more heterologous alpha-1,2 mannosidases and in some embodiments, also retains a fully functional wild type OCH1.

In various embodiments the homogeneity of glycosylation (i.e. the proportion of proteins that carry only Man₅GlcNAc₂ structures on their peptide backbone) can be tuned by controlling the expression of the heterologous mannosidases. In some embodiments, the host microorganism expresses one or more heterologous alpha-1,2 mannosidases. The heterologous alpha-1,2 mannosidases may be of fungal origin, avian origin and/or mammalian origin. The heterologous alpha-1,2 mannosidase is from Trichoderma reesei, such as the MDS2 enzyme with a SEQ ID NO: 7. In some embodiments, the heterologous alpha-1,2 mannosidase is from a chicken such as from Gallus gallus, such as the SEQ Id NO: 150. In other embodiments certain alpha-1,2 Mannosidases chosen from but not limited to those proteins corresponding to SEQ ID Nos 1 to 10 and SEQ ID Nos. 145-150, an amino acid sequence encoded by SEQ ID Nos. 151-152.

In some embodiments, the proteins may have a sequence that has 80%, 85%, or more sequence identity with any of SEQ ID Nos 1 to 10 or SEQ ID Nos. 145-151. In some cases, the sequence identity may be greater than 90%, 95%, 98%. In some embodiments, the proteins may be encoded by a nucleic acid sequence having a sequence that has 80%, 85% or more sequence identity with any of SEQ ID Nos. 152-153. In some cases, the nucleotide sequence identity may be greater than 90%, 95%, 98%. The heterologous mannosidases may be one with more than 70%, 75%, 80%, 85%, 90%, 92%, 95%, 97%, 98%, 99% sequence identity with SEQ ID NO: 7. The heterologous mannosidases may be one with more than 70%, 75%, 80%, 85%, 90%, 92%, 95%, 97%, 98%, 99% sequence identity with SEQ ID NO: 150.

The mannosidases used may be a functional equivalent or functional fragment of an enzyme with any of SEQ ID Nos. 1 to 10 or SEQ ID Nos. 145-151. As used herein “functional fragment” means a polypeptide fragment of an enzyme which substantially retains the enzymatic activity of the full-length protein. A mannosidase may be a substantially equivalent functional fragment of SEQ ID No: 7. A mannosidase may be a substantially equivalent functional fragment of SEQ ID No: 150. By “substantially” is meant at least about 40%, or preferably, at least 50% or more of the enzymatic activity of the full-length α-1,2-mannosidase is retained.

Certain alpha-1,2 mannosidases can have more efficient activity on a target protein than others. In some embodiments, two or more heterologous alpha-1,2 mannosidases are recombinantly expressed. The two or more alpha-1,2 mannosidases may be from the same, similar or different origins.

The combination of two or more interventions described herein can further be used to reduce hypermannosylation of recombinant proteins. For example, one can express recombinant alpha-1,2 mannosidase in a host along with a recombinant protein in a strain that contains a mutation, deletion or otherwise reduced or eliminated expression of OCH1.

In other embodiments the resultant microorganism expressing one or more heterologous alpha-1,2 mannosidases is so designed in order to effect a desired homogeneity and or reduction in the degree of glycosylation of one or more target proteins (chosen from but not limited to those proteins or peptide subsequences corresponding to SEQ ID Nos 11 to 26) also expressed as heterologous proteins in the same microorganism.

In some embodiments herein, recombinant alpha-1,2 mannosidase is expressed in a host along with expressing one or more recombinant proteins. In some embodiments herein, expression of a recombinant alpha-1,2 mannosidase along with expressing one or more recombinant proteins results in a recombinant protein with an improved nutritional value or nutritional content. In some embodiments herein, expression of a recombinant alpha-1,2 mannosidase along with expressing one or more recombinant proteins provides a recombinant protein having a nitrogen to carbon ratio equal to or greater than the protein when isolated from its naturally-occurring source and/or from a different heterologous host. The recombinant protein may be secreted out of the host cell.

The recombinant protein may be a nutritional protein. The nutritional protein may be a protein that contains a desirable amount of essential amino acids. The nutritive protein may comprise at least 30% essential amino acids by weight. The nutritive protein may comprise at least 40% essential amino acids by weight. The nutritive protein may comprise at least 50% essential amino acids by weight. The nutritive protein may comprises or consists of a protein or fragment of a protein that naturally occurs in an edible form. The nutritional protein may be an animal protein. The nutritional protein may be an avian protein. The nutritional protein may be an egg-white protein.

In some embodiments herein, recombinant alpha-1,2 mannosidase is expressed in a host along with expressing one or more egg white proteins. In some embodiments, the proteins or peptides may have a sequence that has 80% or more sequence identity with any of SEQ ID Nos 11 to 26. In some cases, the sequence identity may be greater than 90%, 92%, 95%, 98%.

In some embodiments herein, expression of a recombinant alpha-1,2 mannosidase along with expressing one or more egg white proteins provides an egg white protein with an improved nutritional value. In some embodiments herein, expression of a recombinant alpha-1,2 mannosidase along with expressing one or more egg white proteins provides an egg white protein having a nitrogen to carbon ratio equal to or greater than the egg white protein when isolated from naturally-occurring chicken egg.

A nutritional protein may be produced recombinantly in a host cell which expresses a heterologous mannosidase enzyme in addition to the nutritional protein. Alternatively, a recombinant nutritional protein may be treated with a mannosidase described herein. The resulting recombinant protein may be a reduced glycosylated protein or deglycosylated protein.

Reduced glycosylation or deglycosylation may refer to a reduced size of the carbohydrate moiety on the recombinant glycoprotein, particularly with fewer mannose residues, when the recombinant glycoprotein is expressed in a microorganism which has been modified as described herein as compared to a wild type, unmodified strain of the microorganism. “De-glycosylated” proteins can have a level of N-linked glycosylation that is reduced by at least about 10 percent (e.g., 10 percent, 20 percent, 30 percent, 40 percent, 50 percent, 60 percent, 70 percent, 80 percent, 90 percent, or 100 percent) as compared to the level of N-linked glycosylation of the same proteins that are not produced in the presence of or otherwise exposed to a mannosidase.

The enzymes used to reduce the glycosylation of one or greater proteins may include mannosidases, greater preferably an alpha-1,2 mannosidase. The enzyme may reduce the glycosylation of the recombinant proteins secreted from the host cell. For instance, a fraction of the recombinant protein may be deglycosylated by the enzyme. The enzyme may reduce the glycosylation of greater than 1% of the nutritional protein secreted from the host cell. The enzyme may reduce the glycosylation of greater than 5% of the nutritional protein secreted from the host cell. The enzyme may reduce the glycosylation of greater than 10% of the nutritional protein secreted from the host cell. The enzyme may reduce the glycosylation of greater than 20% of the nutritional protein secreted from the host cell. The enzyme may reduce the glycosylation of greater than 30% of the nutritional protein secreted from the host cell. The enzyme may reduce the glycosylation of greater than 40% of the nutritional protein secreted from the host cell. The enzyme may reduce the glycosylation of greater than 50% of the nutritional protein secreted from the host cell. The enzyme may reduce the glycosylation of greater than 60% of the nutritional protein secreted from the host cell. The enzyme may reduce the glycosylation of greater than 75% of the nutritional protein secreted from the host cell. The enzyme may reduce the glycosylation of greater than 80% of the nutritional protein secreted from the host cell. The enzyme may reduce the glycosylation of greater than 90% of the nutritional protein secreted from the host cell. The enzyme may reduce the glycosylation of greater than 95% of the nutritional protein secreted from the host cell.

The degree of glycosylation or the number of glycan units on a single protein may be modified in the host cell. The degree of glycosylation of the recombinant protein may be less than 90% of the degree of glycosylation of a control protein. The degree of glycosylation of the recombinant protein may be less than 80% of the degree of glycosylation of a control protein. The degree of glycosylation of the recombinant protein may be less than 75% of the degree of glycosylation of a control protein. The degree of glycosylation of the recombinant protein may be less than 50% of the degree of glycosylation of a control protein. The degree of glycosylation of the recombinant protein may be less than 30% of the degree of glycosylation of a control protein. The degree of glycosylation of the recombinant protein may be less than 20% of the degree of glycosylation of a control protein. The degree of glycosylation of the recombinant protein may be less than 15% of the degree of glycosylation of a control protein. The degree of glycosylation of the recombinant protein may be less than 10% of the degree of glycosylation of a control protein. The degree of glycosylation of the recombinant protein may be less than 5% of the degree of glycosylation of a control protein. The degree of glycosylation of the recombinant protein may be less than 1% of the degree of glycosylation of a control protein.

### Compositions Comprising Recombinant Proteins

A consumable composition may comprise one or more recombinant proteins. As used herein, the term “consumable composition” refers to a composition, which comprises an isolated recombinant protein and may be consumed by an animal, including but not limited to humans and other mammals. Consumable food compositions include food products, beverage products, dietary supplements, food additives, and nutraceuticals as non-limiting examples. The consumable composition may comprise one or more components in addition to the recombinant protein. The one or more components may include ingredients, solvents used in the formation of foodstuff, beverages, etc. For instance, the recombinant protein may be in the form of a powder which can be mixed with solvents to produce a beverage or mixed with other ingredients to form a food product.

The nutritional content of the deglycosylated recombinant protein may be higher than the nutritional content of an identical quantity of a control protein. The control protein may be the same protein produced recombinantly but not treated with a mannosidase. The control protein may be the same protein produced recombinantly in a host cell which does not express a heterologous mannosidase. The control protein may be the same protein isolated from a naturally occurring source. For instance, the control protein may be an isolated an egg white protein such as OVD, OVA, or other protein that can be isolated from native egg white.

The nutritional content of a composition comprising the recombinant nutritional protein can be more than the nutritional content of the composition comprising a control protein. The nutritional content may be the protein content of the protein. The protein content of the composition may be about 1% to 80% more than the protein content of a composition comprising a control protein. The protein content of the composition may be about 1% to 5% more than the protein content of a composition comprising a control protein. The protein content of the composition may be about 1% to 10% more than the protein content of a composition comprising a control protein. The protein content of the composition may be about 1% to 20% more than the protein content of a composition comprising a control protein. The protein content of the composition may be about 1% to 50% more than the protein content of a composition comprising a control protein. The protein content of the composition may be about 1% to 80% more than the protein content of a composition comprising a control protein. The protein content of the composition may be about 5% to 10%, 5-15%, 5-20%, 5-30%, 5-50%, 5-80% more than the protein content of a composition comprising a control protein. The protein content of the composition may be about 10% to 80%, 10-20%, 10-30%, 10-50%, 10-70%, 10-80% more than the protein content of a composition comprising a control protein. The protein content of the composition may be about 1%, 5%, 10%, 20%, 30%, 40%, 50%, 60%, 70%, or 80% more than the protein content of a composition comprising a control protein.

Protein content of a composition may be measured using conventional methods. For instance, protein content may be measured using nitrogen quantitation by combustion and then using a conversion factor to estimate quantity of protein in a sample followed by calculating the percentage (w/w) of the dry matter.

The nitrogen to carbon ratio of a deglycosylated protein be higher than the nitrogen to carbon ratio of a control protein. The nitrogen to carbon ratio of a recombinant protein may be greater than or equal to about 0.1. The nitrogen to carbon ratio of a deglycosylated protein be higher than the nitrogen to carbon ratio of a control protein. The nitrogen to carbon ratio of a recombinant protein may be greater than or equal to about 0.25. The nitrogen to carbon ratio of a recombinant protein may be greater than or equal to about 0.3. The nitrogen to carbon ratio of a recombinant protein may be greater than or equal to about 0.35. The nitrogen to carbon ratio of a recombinant protein may be greater than or equal to about 0.4. The nitrogen to carbon ratio of a recombinant protein may be greater than or equal to about 0.5.

Solubility of a deglycosylated protein may be greater than the solubility of a control protein. Solubility of a composition comprising a deglycosylated protein may be higher than the solubility of a composition comprising the control protein. Thermal stability of the deglycosylated protein may be greater than the thermal stability of a control protein.

The degree of glycosylation of the recombinant protein may be dependent on the consumable composition being produced. For instance, a consumable composition may comprise a lower degree of glycosylation to increase the protein content of the composition. Alternatively, the degree of glycosylation may be higher to increase the solubility of the protein in the composition.

### A Microorganism Carrying a Heterologously Expressed Alpha-1,2 Mannosidase

The following outlines the construction of a microorganism expressing a heterologous alpha-1,2 mannosidase.

Herein an “alpha-1,2 mannosidase” refers to any protein that recognized as catalyzing the cleavage of an alpha-1,2 glycosidic bond between mannose groups in a glycan structure that contains ManₓGlcNAc₂ (where x>=6) as a substructure (with reference to bonds illustrated in FIG. 1). Examples of alpha-1,2 mannosidase to those proteins encoded by any of the polynucleotide sequences or subsequences therein represented in the list comprised of SEQ ID Nos 1 to 10 and SEQ ID Nos. 145-151 or encoded by SEQ ID Nos. 152-153.

In eukaryotic organisms, precursor oligosaccharides structures (Glc₃Man₉GlcNAc₂) synthesized in the Endoplasmic Reticulum (ER) can be added to asparagine residues of a polypeptide (at consensus Asn-X-Ser or Asn-X-Thr or Asn-X-Cys sites where X is any amino acid except a Proline) in the first step of what is known as N-glycosylation. In the lumen of the ER, the precursor oligosaccharide is cleaved to remove the glucose residues of each attached Glc₃Man₉GlcNAc₂ oligosaccharide (FIG. 1A). The additional removal of a mannose group results in a Man₈GlcNAc₂ core structure (FIG. 1B). This core structure is further processed upon translocation of the glycoprotein to the Golgi. In yeast Golgi, this processing involves the activity of OCH1, an alpha-1,6 mannosyltransferase that acts on Man₈GlcNAc₂ core structures in a step necessary to initiate the further addition of mannosyl groups that can ultimately give rise to hypermannosylated glycan groups on the fully processed protein. (FIG. 1D) illustrates Man₅GlcNAc₂, a possible product upon cleavage of Man₈GlcNAc₂ at alpha-1,2 glycosidic bonds by an alpha-1,2 mannosidase. Unlike Man₈GlcNAc₂, OCH1 does not carry out efficient alpha-1,6 mannosyltransferase activity on Man₅GlcNAc₂ as a substrate. Triangle—glucose; square—N-acetylglucosamine; circle-Mannose.

Herein a “transformation” of a microorganism refers to the introduction of polynucleotides into a microorganism.

Herein a “transformant” refers to a microorganism that has been transformed.

Herein a “transgene” refers to a polynucleotide that can form a gene product if contained in a microorganism.

Herein an “expression cassette” is any polynucleotide that contains a subsequence that codes for a transgene and can confer expression of that subsequence when contained in a microorganism and is heterologous to that microorganism.

Herein a “promoter” refers to a polynucleotide subsequence of an expression cassette that is located upstream or 5′ to a transgene and is involved in initiating transcription from that transgene when the expression cassette is contained in a microorganism.

Herein a “glycoprotein” refers to a protein that carry carbohydrates covalently bound to their peptide backbone.

Herein a “glycoform” refers to any of several different forms of a glycoprotein where each is differentiated from the other by the different structures of peptide-bound polysaccharides.

In some embodiments the host microorganism carries one or more stably integrated heterologous transgenes that when expressed as proteins in the host are intended targets for alterations of their glycan groups by the heterologous alpha-1,2 mannosidase. Herein such transgenes are referred as the “target proteins”.

A. Synthesis of Vectors Containing Expression Cassettes:

First a vector carrying an expression cassette, containing an alpha-1,2 mannosidase to be transformed is made. In some embodiments multiple different alpha-1,2 mannosidases could be transformed, either on vectors carrying multiple expression cassettes, or on separate vectors. The expression cassettes described herein can be obtained using chemical synthesis, molecular cloning or recombinant methods, DNA or gene assembly methods, artificial gene synthesis, PCR, or any combination thereof. Methods of chemical polynucleotide synthesis are well known in the art and need not be described in detail herein. One of skill in the art can use the sequences provided herein and a commercial DNA synthesizer to produce a desired DNA sequence. For preparing polynucleotides using recombinant methods, a polynucleotide comprising a desired sequence can be inserted into a suitable cloning or expression vector, and the cloning or expression vector in turn can be introduced into a suitable host cell for replication and amplification. Suitable cloning vectors may be constructed according to standard techniques, or may be selected from a large number of cloning vectors available in the art. While the cloning vector selected may vary according to the host cell intended to be used, useful cloning vectors will generally may the ability to self-replicate, may possess a single target for a particular restriction endonuclease, and/or may carry genes for a marker that can be used in selecting clones containing the expression vector. Methods for obtaining cloning and expression vectors are well-known (see, e.g., Green and Sambrook, Molecular Cloning: A Laboratory Manual, 4th edition, Cold Spring Harbor Laboratory Press, New York (2012)).

FIG. 2 provides examples of a vectors created by these means; FIG. 2 describes a vector containing (A) a promoter (FBA1 promoter in FIG. 2) operably linked to a transgene (T. reesei alpha-1,6 mannosidase 1—T.R. MDS1 in FIG. 2). The vector further comprises a C-terminus sequence encoding an HDEL ER retention signal fused in frame with the transgene (HDEL FIG. 2). The vector further comprises a Terminator Element (AOX1 terminator in FIG. 2). These elements are collectively referred to herein as an “Expression Cassette”, although in some embodiments a signal peptide can also be included in the design. In some embodiments the ER retention signal may or may not be present. To aide in the amplification of the vector prior to transformation into the host microorganism, those skilled in the art may rely on a replication origin (E) contained in the vector (ORI in FIG. 2). To aide in the selection of a microorganism stably transformed with the expression vector from those microorganisms that don't contain the expression vector, those skilled in the art may rely on a selection marker (F) contained in the vector downstream of a promoter element (Zeocin resistance gene in FIG. 2) The expression vector can also contain a restriction enzyme site (G) (SwaI in FIG. 2) that allows for linearization of the expression vector prior to transformation into the host microorganism to facilitate the expression vectors stable integration into the host genome. In FIG. 2, elements E,F may be removed from their genomic location post transformation by one skilled in the art due to the presence flanking LoxP sites that can catalyze excision of the intervening region by the CRE/lox recombination (https://en.wikipedia.org/wiki/Cre-Lox recombination). In general, the expression cassette is designed to mediate the transcription of the transgene when integrated into the genome of a cognate host microorganism. For the elements comprising the expression vectors in FIG. 2, this host microorganism is Pichia Pastoris although in other embodiments this host organism can be any microorganism where one skilled in the art can introduce the expression vector into its genome such that the elements in the expression vector are recognized by the cell to sufficiently induce the transcription and subsequent processing of transcript into the intended full-length protein. In some embodiments the transgene may be codon optimized for optimal expression in the host organism.

The genetic elements of the expression vector can be designed to be suitable for expression in the intended microorganism host by one trained in the art. In some embodiments an additional vector and or additional elements may be designed to aide (as deemed necessary by one skilled in the art) for the particular method of transformation (e.g. CAS9 and gRNA vectors for a CRISPR/CAS9 based method).

The Promoter Element (A) may include, but is not limited to, a constitutive promoter, inducible promoter, and hybrid promoter. Promoters include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, inv1+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PETS, peroxin 8 (PEX8), phosphoglycerate kinase (PGK, PGK1), pho1, PHO5, PH089, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha-(TEF1), THI11, homoserine kinase (THR1), tpi, TPS1, triose phosphate isomerase (TPI1), XRP2, YPT1, GCW14, GAP, a sequence or subsequence chosen from SEQ ID Nos: 31 to 47, and any combination thereof. In some embodiments, the nucleotides used may have a sequence that has 80% or more sequence identity with any of SEQ ID Nos 31 to 47. In some cases, the sequence identity may be greater than 90%, 95%, 98%.

A promoter used to express the mannosidases described herein may be heterologous to the host cell. A promoter used to express the mannosidases described herein may be native to the host cell. A promoter used to express the mannosidases described herein may be constitutive or inducible. A strong promoter may be used to drive the expression of the α-1,2-mannosidase. For instance, if a higher protein content is desired, the vector may comprise a strong promoter to increase the degree of deglycosylation of the recombinant protein. Alternatively, a weaker promoter may be used to drive the expression of the α-1,2-mannosidase. For instance, if a lower degree of deglycosylation is required, a weaker promoter may be used to drive the expression of the mannosidase.

A host cell may comprise a first promoter driving the expression of the recombinant nutritional protein and a second promoter driving the expression of the α-1,2-mannosidase. The first and second promoter may be selected from the list of promoters provided herein. In some cases, the expression of α-1,2-mannosidase and the recombinant nutritional protein may be derived from the same promoters. Alternatively, the first and the second promoter may be different.

The Signal peptide (B) A signal peptide, also known as a signal sequence, targeting signal, localization signal, localization sequence, signal peptide, transit peptide, leader sequence, or leader peptide, may support secretion of a protein or polynucleotide. Extracellular secretion of a recombinant or heterologously expressed protein from a host cell may facilitate protein purification. A signal peptide may be derived from a precursor (e.g., prepropeptide, preprotein) of a protein. Signal peptides may be derived from a precursor of a protein including, but not limited to, acid phosphatase (e.g., Pichia pastoris PHO1), albumin (e.g., chicken), alkaline extracellular protease (e.g., Yarrowia lipolytica XRP2), α-mating factor (α-MF, MATa) (e.g., Saccharomyces cerevisiae), amylase (e.g., α-amylase, Rhizopus oryzae, Schizosaccharomyces pombe putative amylase SPCC63.02c (Amyl)), β-casein (e.g., bovine), carbohydrate binding module family 21 (CBM21)-starch binding domain, carboxypeptidase Y (e.g., Schizosaccharomyces pombe Cpy1), cellobiohydrolase I (e.g., Trichoderma reesei CBH1), dipeptidyl protease (e.g., Schizosaccharomyces pombe putative dipeptidyl protease SPBC1711.12 (Dpp1)), glucoamylase (e.g., Aspergillus awamori), heat shock protein (e.g., bacterial Hsp70), hydrophobin (e.g., Trichoderma reesei HBFI, Trichoderma reesei HBFII), inulase, invertase (e.g., Saccharomyces cerevisiae SUC2), killer protein or killer toxin (e.g., 128 kDa pGKL killer protein, α-subunit of the K1 killer toxin (e.g., Kluyveromyces lactis), K1 toxin KILM1, K28 pre-pro-toxin, Pichia acaciae), leucine-rich artificial signal peptide CLY-L8, lysozyme (e.g., chicken CLY), phytohemagglutinin (PHA-E) (e.g., Phaseolus vulgaris), maltose binding protein (MBP) (e.g., Escherichia coli), P-factor (e.g., Schizosaccharomyces pombe P3), Pichia pastoris Dse, Pichia pastoris Exg, Pichia pastoris Pir1, Pichia pastoris Scw, and cell wall protein Pir4 (protein with internal repeats). Examples of signal peptides can also comprise a sequence or subsequence chosen from SEQ ID Nos 48 to 144, and any combination thereof. In some embodiments a signal peptide is not present. In some embodiments, the signal proteins or peptides may have a sequence that has 80% or more sequence identity with any of SEQ ID Nos 48 to 144. In some cases, the sequence identity may be greater than 90%, 95%, 98%.

### ER Targeting/Retention Signal

This motif will signal the retention of the resultant protein to the ER. An ER retention signal may be derived from a precursor (e.g., prepropeptide, preprotein) of a protein. ER retention signals may be derived from a precursor of a protein including, but not limited to, polynucleotides that encode the amino acid sequence KDEL, HDEL, or transmembrane domains that may be encoded by subsequences contained in SEQ ID Nos 1 to 10 or 145 to 149. The ER retention signal is typically fused in frame on the C-terminus of the transgene ORF, although in some embodiments it may be fused in frame on the transgene N-terminus immediately downstream of the cleavage site of the signal peptide if it is present. In some embodiments an ER retention signal is not present. In some embodiments, the expressed protein, such as an alpha-1,2 mannosidase, will be retained in the ER or otherwise not require an ER retention signal to provide intracellular deglycosylation of a heterologous protein.

The Transgene (C) may include, but is not limited to, nucleic acids encoding polypeptides such as those polynucleotides chosen from the list comprised of SEQ ID Nos: 1 to 30 or 145 to 150. These sequences can be designed to be altered to encode the same protein, and be optimized for expression in the chosen host (i.e. codon optimized); for example, the nucleic acid sequence encoding an alpha-1,2 mannosidase and a codon optimized form SEQ ID Nos. 151-152.

The Terminator Element (D) in this example is the AOX1 terminator, but it may chosen to be any suitable sequences that serves to abort continuing elongation of the nascent transcript containing the mRNA corresponding to the transgene.

The Selectable Marker (F) may include, but is not limited to: an antibiotic resistance gene (e.g. zeocin, ampicillin, blasticidin, kanamycin, nurseothricin, chloroamphenicol, tetracycline, triclosan, ganciclovir, and any combination thereof), an auxotrophic marker (e.g. f ade1, arg4, his4, ura3, met2, and any combination thereof).

Transformation of Microorganism Host with Vectors

Next, expression vectors or polynucleotides (DNA or RNA) containing genetic information encoding expression cassettes derived from expression vectors are inserted into host cells and clonal populations of successful transformants may be isolated by any means known in the art.

Microorganisms that are suitable for transformation with a polynucleotide carrying an expression cassette that contains a subsequence that encodes for an alpha-1,2 mannosidase by someone trained in the art. These can include but are not limited to: Arxula spp., Arxula adeninivorans, Kluyveromyces spp., Kluyveromyces lactis, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia hpolytica, Agaricus spp., Agaricus bisporus, Aspergillus spp., Aspergillus awamori, Aspergillus fumigatus, Aspergillus nidulans, Aspergillus niger, Aspergillus oryzae, Colletotrichum spp., Colletotrichum gloeosporiodes, Endothia spp., Endothia parasitica, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculosum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, Trichoderma vireus, Aspergillus oryzae, Bacillus subtilis, Escherichia coli, Myceliophthora thermophila, Neurospora crassa, Pichia pastoris, Komagatella phaffii and Komagatella pastoris.

Cells may be transformed by introducing an exogenous polynucleotide, for example, by direct uptake, endocytosis, transfection, F-mating, PEG-mediated protoplast fusion, Agrobacterium tumefaciens-mediated transformation, biolistic transformation, chemical transformation, or electroporation. Once introduced, the exogenous polynucleotide can be maintained within the cell as a non-integrated expression vector (such as a plasmid) or integrated into the host cell genome. The cell population can be selected for those cells that take up the exogeneous expression vectors (by virtue of resistance genes carried on the expression vectors) by plating onto agar plates containing some agent (e.g. the antibiotic Zeocin) that negatively selects cells that are not carrying a gene conferring resistance to that agent.

Alternatively, one can create an auxotrophic strain by knocking out a gene (e.g. URA3 gene in Pichia pastoris) required for synthesis of an essential metabolite (e.g. uracil), transform this strain using expression vectors that contain as a selection marker a gene that complements the knock out (i.e. the URA3 gene) and select for transformed cells by virtue of their ability to grow on a media that lacks this essential metabolite.

With either approach after incubating plates that have been spread with a population of cells containing putative transformants for time and temperature appropriate for growth of colonies that can be manually selected (as known to one trained in the art), individual colonies can be picked and verified for the integration of expression vectors into the host cell genome by standard molecular biological methods that are known to one trained in the art (i.e. colony PCR, genomic sequencing). Individual colonies from these plates can then be used to inoculate individual culture vessels containing appropriate growth medium for the cell line containing a selection agent chosen as appropriate for the selection marker(s) contained in the transformed expression vectors. After an appropriate amount of time (e.g. overnight at 30 degrees Celsius in a shaker flask; otherwise known to one trained in the art) The successful transformation of a cell line with recombinant vector can be determined in each culture vessel by the presence of protein coded by the transgene on the transformed expression cassettes (referred to henceforth as “recombinant protein”). This expression can be determined by standard molecular biology methods (e.g. Western blot, SDS-PAGE with known standard protein). Colonies from those plates that correspond to culture vessels that show the recombinant protein expression can then be used to inoculate vessels containing selection media appropriate for the transformed cell line to promote growth of the cell line and expression of the recombinant protein. Alternatively, colonies from those plates that correspond to culture vessels that showed recombinant protein expression can be stored for later use (e.g. at −80 degrees Celsius in a glycerol stock).

### Determination of Efficacy of Transformed Strain

Resultant strains confirmed to be stably transformed with an integrated transgene encoding an alpha-1,2 mannosidase are tested for the effect of its expression on the glycosylation of either endogenous or heterologously expressed target proteins.

The expression and purification of proteins expressed in parental wild type strains or parental strains that contain a heterologous alpha-1,2 mannosidase are known to one trained in the art. For example, in a methylotrophic yeast strain (such as Pichia Pastoris) a target protein can be induced if it is operably linked to a methanol induced promoter (i.e. AOX1) for strong over expression. If this target protein also contains a signal peptide it can be recovered from the media, and be sufficiently purified for analysis using techniques known to one trained in the art. In general, one can compare the glycan groups present on a protein of interest (e.g. the target proteins) between protein samples purified from cells with and without (herein referred to as the “control proteins”) the alpha-1,2 mannosidases or as compared to the the same protein isolated from a native source. Such measures of sample preparation and comparison can be carried out using techniques included, but not limited to methods such as: capillary electrophoresis or SDS-PAGE for size comparison of protein of interest, immunostaining techniques (e.g. Western blotting) using glycan specific antibodies, and quantitative mass spectrometry methods to identify glycan groups within a sample (e.g. N-linked glycan profiling by MALDI-TOF/TOF MS). See, e.g., Ziv Roth, Galit Yehezkel, and Isam Khalaila International Journal of Carbohydrate Chemistry Volume 2012 (2012).

In some embodiments, a ratio for ManₓGlcNAc₂ and Man_{y}GlcNAc₂ values may be calculated for a recombinantly expressed egg white protein. In some cases, the x value may be less than or equal to 1, 2, 3, 4 or 5. In some cases, they value may be greater than or equal to 6, 7, 8, 9 or 10. In some cases, the ratio of ManₓGlcNAc₂:Man_{y}GlcNAc₂ may be greater than 1. In some embodiments, a recombinantly expressed egg white protein may have a degree of polymerization that is less than or equal to 9. In some cases, the degree of polymerization may be less than 9, 8, 7 or 6.

The following example outlines the preparation and analysis of samples for determining the glycan groups present on a target protein (namely the protein corresponding to SEQ ID NO: 12). In some embodiments, the target proteins or peptides may have a sequence that has 80% or more sequence identity with any of SEQ ID No. 12. In some cases, the sequence identity may be greater than 90%, 95%, or 98%.

In some embodiments, the recombinant egg white protein may have a nitrogen to carbon (N to C) ratio greater than 0.25. In some cases, the N to C ratio for the recombinantly expressed protein may be greater than about 0.25, about 0.3, about 0.35 or about 0.4.

N-Linked Glycan Profiling by MALDI-TOF/TOF MS

An aliquot of each sample corresponding to 300 μg can be used for analysis. The glycoprotein is reduced, alkylated, then digested with trypsin in Tris-HCl buffer overnight. After protease digestion, the sample is passed through a C18 sep pak cartridge, washed with a low w/w percentage acetic acid and the glycopeptides are eluted with a blend of isopropanol in low concentration acetic acid, before being dried by SpeedVac. The dried glycopeptides eluate are treated with PNGase F to release the N-linked glycans and the digest is passed through a C18 sep pak cartridge to recover the N-glycans.

Per-O-Methylation of N-Linked Glycans

The N-linked glycans is permethylated for structural characterization by mass spectrometry (Anumula and Taylor, 1992). Briefly, the dried eluate is dissolved with dimethyl sulfoxide and methylated with NaOH and methyl iodide. The reaction is quenched with water and per-O-methylated carbohydrates is extracted with methylene chloride and dried under N₂.

Profiling by Matrix-Assisted Laser-Desorption Time-of-Flight Mass Spectrometry (MALDI-TOF/TOF MS)

The permethylated glycans is dissolved with methanol and crystallized with α-dihyroxybenzoic acid (DHBA) matrix. Analysis of glycans present in the samples is performed by MALDI-TOF/TOF-MS using AB SCIEX TOF/TOF 5800 (Applied Biosystems).

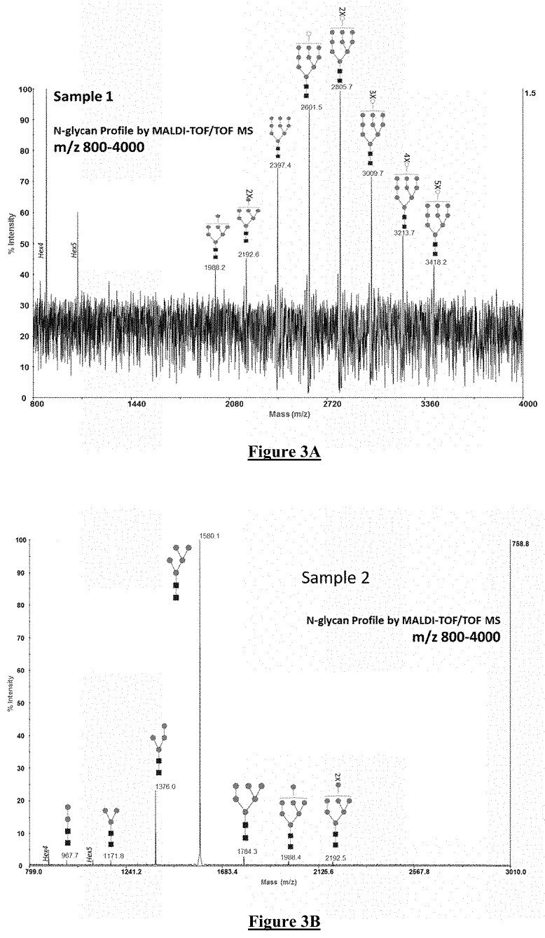

FIGS. 3A and 3B illustrate a sample mass spectra results from the above procedure, intended to inform the practitioner of the relative amounts of each glycoform present in a control sample (FIG. 3A) relative to a sample obtained from a cell line expressing a heterologous alpha-1,2 mannosidase (FIG. 3B). The relative amounts for each identified glycoform are laid out in Tables 1 and 2 corresponding to the control sample and alpha-1,2 mannosidase sample respectively. The data presented in this figure represents a prophetic result in which the activity of the mannosidase is effecting an increase in the relative presence of Man₅GlcNAc₂ type structures relative to other glycan structures within the sample relative to the control sample. In sample 2, Man₅GlcNAc₂ comprises 77.1% of identified glycoforms (Table 1), while in sample 1, Man₅GlcNAc₂ is not represented among the identified glycoforms (Table 2). Square—N-acetylglucosamine (GlcNac); green circle Mannose (Man); white circle—Hexose (Hex).

TABLE 1
N-linked glycans from Sample 1 (rOVD expressed in Pichia) detected by
MALDI TOF/TOF MS.

| Permethylated | Text description of | Cartoon representation | |
|---|---|---|---|
| mass (m/z)¹ | structures | of possible structures | Percentage |
| 1988.0 | Man₇GlcNAc₂ | 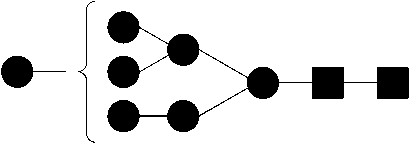 | 8.0 |
| 2192.1 | Man₈GlcNAc₂ | 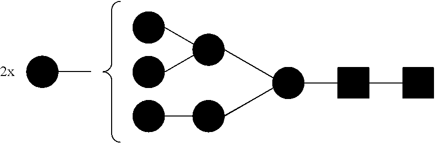 | 8.6 |
| 2396.2 | Man₉ GlcNAc₂ | 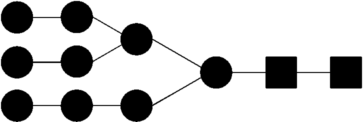 | 14.2 |
| 2600.3 | Man₉ GlcNAc₂ Hex | 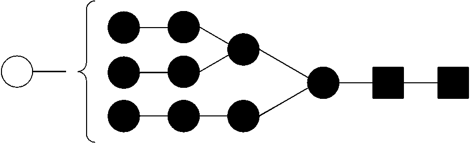 | 17.8 |
| 2804.4 | Man₉ GlcNAc₂Hex₂ | 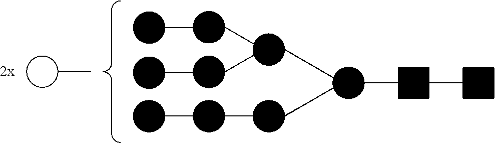 | 18.9 |
| 3008.5 | Man₉ GlcNAc₂Hex₃ | 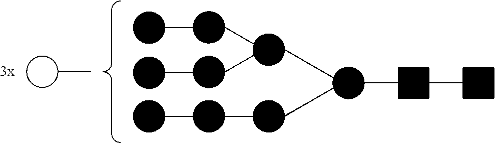 | 13.7 |
| 3212.6 | Man₉ GlcNAc₂Hex₄ | 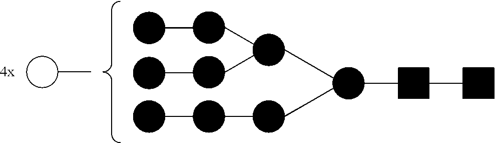 | 10.0 |
| 3416.7 | Man₉ GlcNAc₂Hex₅ | 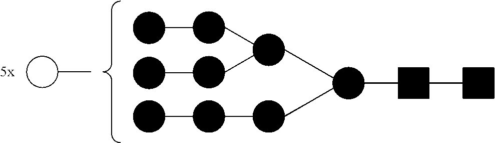 | 8.7 |

¹All masses (mass + Na) are single-charged.
²Calculated from the area units of detected N-linked glycans.

TABLE 2
N-linked glycans from Sample 2 (rOVD expressed in a modified Pichia strain)
detected by MALDI TOF/TOF MS.

| Theoretical | | | |
|---|---|---|---|
| Permethylated | Text description of | Cartoon representation | |
| mass (m/z)¹ | structures | of possible structures | Percentage |
| 967.5 | Man₂GlcNAc₂ | 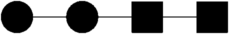 | 1.4 |
| 1171.6 | Man₃GlcNAc₂ | 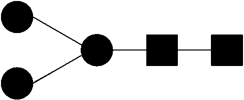 | 1.7 |
| 1375.7 | Man₄GlcNAc₂ | 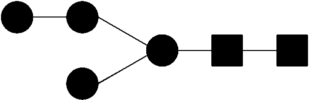 | 15.4 |
| 1579.8 | Man₅GlcNAc₂ | 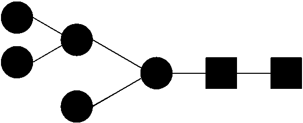 | 77.1 |
| 1783.9 | Man₆GlcNAc₂ | 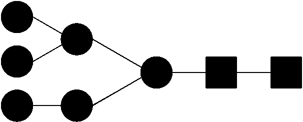 | 2.3 |
| 1988.0 | Man₇GlcNAc₂ | 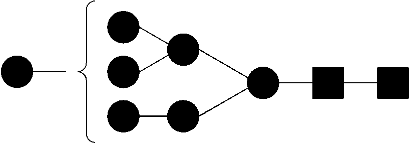 | 1.1 |
| 2192.1 | Man₈GlcNAc₂ | 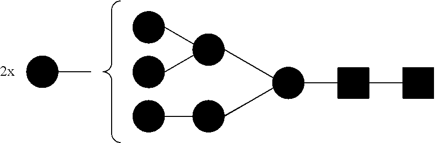 | 1.1 |

### EXAMPLES

### Example 1: Identification of alpha-1,2 mannosidases

Blast P was used to search for protein sequences with identity to known alpha-1,2 mannosidases that could confer modification of the glycan structures on proteins expressed heterologously in Pichia sp. (currently reclassified as Komagataella species). Exemplary fungal alpha-1,2 mannosidase protein sequences identified including SEQ ID Nos. 1-10. A further search was performed for sequences in Gallus gallus. Exemplary Gallus gallus alpha-1,2 mannosidase protein sequences include SEQ ID Nos. 145-150.

### Example 2: Construction of Expression Vectors for Alpha-1,2 Mannosidase Expression in Pichia

A fungal alpha-1,2 mannosidase protein sequence, SEQ ID NO. 7 (referred to as TrMDS2), was selected for expression, along with a Gallus gallus alpha-1,2 mannosidase protein sequence, SEQ ID NO. 150 (referred to as GgMAN1A1). For GgMAN1A1, the cDNA (SEQ ID NO. 152) was codon optimized to increase expression in Pichia (SEQ ID NO. 153, referred to as GgMAN1A1C).

Each cDNA, TrMDS2 and GgMAN1A1C was cloned into a Pichia expression vector downstream of a methanol inducible promoter, the vectors containing the selectable marker for zeocin resistance, The alpha-1,2 mannosidase expression vectors were transformed by electroporation into a K. phaffii strain (Strain 1) previously confirmed to be secreting OVD. Expression cassettes for the 2 alpha-1,2 mannosidase enzymes were transformed both individually and together into the OVD-expressing strain. Transformed cells were selected on zeocin containing agar plates and individual colonies were grown up in a microtiter 96 well plate format to evaluate quality of secreted OVD.

### Example 3: Expression of Alpha-1,2 Mannosidase in Pichia

Bradford protein assays were conducted in a high throughput format to confirm presence of secreted protein in the growth media. The supernatant from select wells were then screened by SDS-PAGE. Clones displaying desired protein patterns from SDS-PAGE were then scaled up in 40 mL shake flask format and/or up to 40 L bioreactor to confirm activity of transformed deglycosidase. External glycan analysis by LC/MS was conducted on one strain expressing TrMDS2 (Strain 2) using material generated in shake flask format. Inspection of SDS-PAGE results from TrMDS2-expressing Pichia indicated that this heterologous protein was not secreted under the conditions tested. This means that the native TrMDS2 protein sequence contains intracellular localization signals that were recognized by Pichia. TrMDS2 protein is large enough that it would run well above OVD and should be visible on the protein gel.

### Example 4: Activity Analysis of Heterologous Expression of TrMDS2 in Pichia

Heterologous expression of TrMDS2 in Strain 2 did not significantly reduce OVD expression compared to its parent strain Strain 1 in shake flask experiments. In its initial shake flask run, SF17, Strain 2 made 95% secreted OVD compared to the average secretion level of a Strain 1 duplicate (FIG. 4A). However, this difference is within the error of shake flask experiments. In a subsequent run, SF22, a duplicate of Strain 2 made 109% secreted OVD compared to a duplicate of Strain 1 (FIG. 4B).

In all experiments, Strain 2 produced a visible band pattern downshift in the secreted OVD as seen by SDS-PAGE analysis (FIGS. 4A-B). This band shift indicated a decrease in the apparent molecular weight of OVD from Strain 1 to Strain 2, theorized to be a result of reduction in glycan presence on the protein.

The reduction of OVD glycosylation in the Strain 2 strain was confirmed by external LC/MS (Table 3). Almost all glycans found on Strain 1 produced OVD have a branch pattern of 9 mannose or more. In contrast, the majority of glycans found on Strain 2 produced OVD contain branches of 8 mannose or less. The known branching patterns of K. phaffii mannosylation are shown in FIG. 5.

TABLE 3
Summary of relative distribution of glycans found on OVD secreted by Strain 1 and Strain 2.
Glycosylation Fragment Distribution

| | Man16 | Man15 | Man14 | Man13 | Man12 | Man11 | Man10 | Man9 | Man8 | Man7 | Man6 | Man5 |
|---|---|---|---|---|---|---|---|---|---|---|---|---|
| STRAIN1 | 2 | 4 | 4 | 6 | 8 | 10 | 8 | 4 | 0 | 0 | 1 | 0 |
| STRAIN2 | 1 | 0 | 1 | 0 | 3 | 2 | 3 | 3 | 7 | 3 | 11 | 12 |

### Example 5: Heterologous Expression of GgMAN1A1 in Pichia

Heterologous expression of GgMAN1A1 in Strain 1 produce a range of deglycosylation effect, the strongest of which approach the band pattern of Strain 2, the weakest of which approximate Strain 1 band pattern with a very slight downshift.

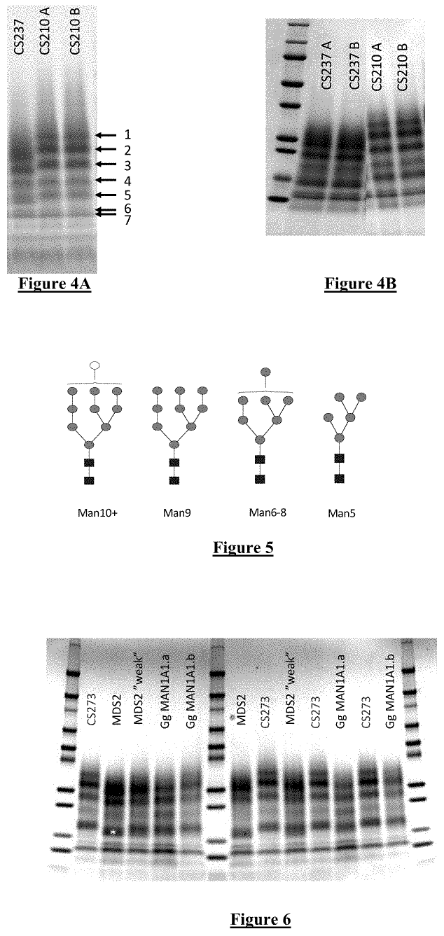

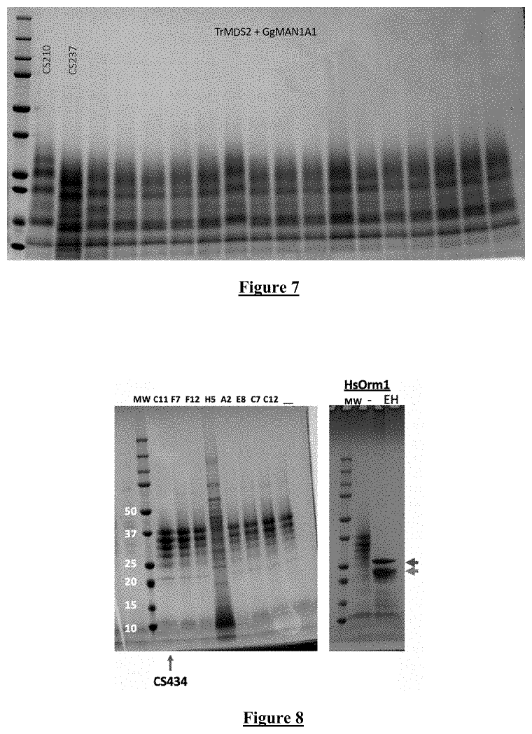

SDS-PAGE analysis was conducted to compare the two extremes of GgMAN1A1 functionality with TrMDS2 as well as Strain 1 pattern (FIG. 6). In the analysis, Strain 3, a derivative strain of Strain 1 making more OVD but maintaining the same glycosylation pattern, was used as the standard OVD band pattern. While TrMDS2 expression varied between transformants, the weaker TrMDS2 clones still showed band patterning very close to that of Strain 2. A “weak” MDS2 clone was included in the comparison in FIG. 6 as well. There were minute differences in the band patterning of TrMDS2 vs GgMAN1A1.

### Example 6: Localization of GgMAN1A1 in Pichia

The sample GgMAN1A1.a represents the strongest deglycosylation effect found during screening, and GgMAN1A1.b represents the weakest. There is a progressive upward band shift from MDS2 to GgMAN1A1.b on the left side of the gel, indicating a range of deglycosylation function. Each sample is then compared to Strain 3 individually on the right side of the gel to confirm deglycosylation. Inspection of SDS-PAGE results from GgMAN1A1-expressing Pichia indicated that this heterologous protein was not secreted under the conditions tested. GgMAN1A1 protein is large enough that it would run well above OVD and should be visible on the protein gel. This means that the native GgMAN1A1 protein sequence contains intracellular localization signals that were recognized by Pichia.

The major difference between the strong and weak TrMDS2 deglycosylation is seen in the band marked by an asterisk. This band appears to be a close doublet. In the strong TrMDS2 pattern, the doublet favors the bottom band, while the weak TrMDS2 pattern favors the top band. GgMAN1A1.a displays a band pattern close to that of MDS2, with the exception of the asterisk-marked band. This band in GgMAN1A1.a appears to be sized between the doublet. GgMAN1A1.b displays a further upward shift of all the bands. When compared immediately next to the standard OVD pattern on the right side of the gel, it is very slightly downshifted and displays the characteristic disappearance of the topmost band seen in TrMDS2 deglycosylated patterns.

TrMDS2 and GgMAN1A1 were coexpressed in Strain 1 and the glycosylation patterns examined by SDS-PAGE analysis. A range of deglycosylation patterns were seen, including that of TrMDS2 alone. (FIG. 7).

### Example 7: Deglycosylation of HsORM1

Human serum glycoprotein, “Orosomucoid 1” (Homo sapiens ORM1; HsORM1; uniport P02763) possesses five predicted N-glycosylation consensus motifs at asparagine residues 33, 56, 72, 93 and 103. An HsORM1 coding sequence was placed downstream of a methanol-inducible promoter. An alpha-mating factor signal sequence was fused to the N-terminus of the HsORM1 coding sequence. The translated fusion provided the polypeptide sequence SEQ ID NO: 154 (bold indicating the HsORM1 sequences and the non-bolded indicating the signal sequence amino acids).

The expression construct was transformed into a Pichia pastoris (also referred to as K. phaffii) mutS strain, primary transformants were selected and then subjected to a 96 h time course using methanol as an inducer of HsORM1 transcription. Expression was analyzed by SDS-polyacrylamide gel electrophoresis (SDS-PAGE) of culture supernatants. Pichia-expressed HsOrm1 migrated as six distinct polypeptide species (see FIG. 8, below); the lowest molecular weight species (21.5 kDa) is predicted to be the non-glycosylated form, and the other forms likely correspond to mono- through penta-glycosylated forms. To demonstrate that Pichia expressed HsORM1 possesses high mannose glycans, the HsOrm1-containing supernatant from Strain 4 was treated in vitro with 1000 units of Endoglucanase H (EH) for 1 h at 37° C. Following EH treatment, the sample was analyzed by SDS-PAGE and only the fully deglycosylated 21.5 kDa polypeptide species remained, further supporting the observation that this is the fully de-glycosylated form.

FIG. 8: Left panel—MW is a molecular weight protein reference ladder; the lanes to the right of MW are individual transformants expressing HsORM1. Right panel—lane 1 is the molecular weight protein reference ladder; lane 2 is an extract of a transformant expressing HsOrm1; lane 3 is extract of the same transformant treated with endoglycosidase H. Black arrow indicates exogenously added Endo H enzyme; grey arrow indicates in vitro deglycosylated HsOrm1 protein species at 21.5 kDa.

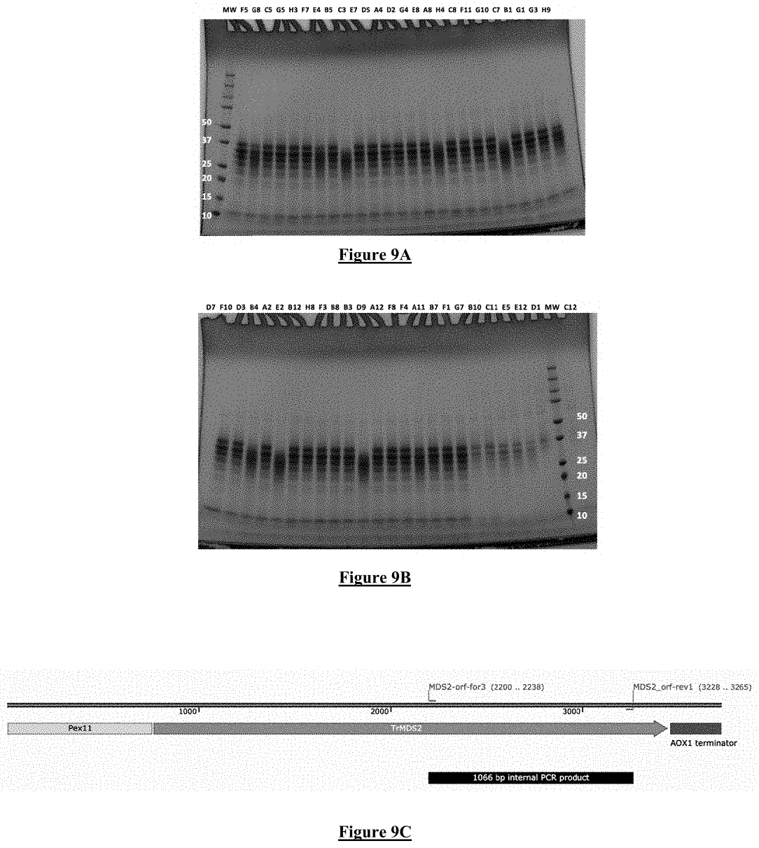

Following strain purification, Strain 4 (corresponding to well C11 supernatant; red arrow above) was made competent for DNA electroporation and subsequently transformed with the TrMDS2 cDNA expression construct under control of the methanol inducible promoter (SEQ ID NO: 38) and a methanol-inducible transcriptional terminator. HsORM1⁺/Pex11-TrMDS2 co-expressors were selected for by their HsORM1 band-shifting patterns following a 96 h time course experiment in methanol-containing induction media. FIGS. 9A and 9B show the banding pattern of HsORM1 on SDS-PAGE of the putative TrMDS2 transformants.

For a subset of the above tested transformants, the presence of TrMDS2 was verified by PCR using primers to amplify an internal 1066 bp PCR product in the open reading frame, as shown in FIG. 9C.

PCR produced a 1066 bp product is all of the tested transformants A2, A8, B3, C3, C7, D3, E4, F4, G8, whereas the PCR product was not found in an untransformed control.

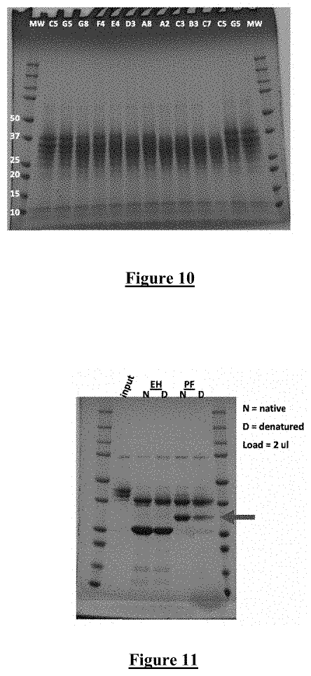

Following the initial induction experiments, a subset of the HsORM1+/TrMDS2 co-expressors were compared for degree of HsORM1 deglycosylation (FIG. 10 below. From left to right, PCR-genotyped strains (positive for the TrMDS2 construct) displayed varying levels of HsOrm1 deglycosylation from very slight to significant deglycosylation, as observed by the increase in smaller HsORM1 polypeptide species on SDS-PAGE. The comparison of these strains indicated that the extent of deglycosylation of an expressed animal protein (such as HsOrm1) can be fine-tuned by selection of a variety of levels of deglycosylation patterns, such as created by differing levels of TrMDS2 expression.

### Example 8: Deglycosylation of Ovalbumin (OVA)

Native G. gallus ovalbumin (OVA) is post-translationally modified by asparagine-linked (N-linked) glycosylation at amino acid residue 292 (SEQ ID NO: 26 in BOLD font) and it has also been noted in the literature that amino acid residue 311 is occasionally glycosylated (SEQ ID NO: 26 BOLD/underlined font).

An OVA expression construct was made containing the Pichia codon-biased ovalbumin cDNA under transcriptional control of an a methanol inducible promoter and a methanol-inducible terminator. This multicopy expression construct was subsequently transformed into a mutS Pichia strain Strain 5 to create Strain 6. Pichia strain Strain 6 was then subjected to antibiotic resistance marker (ARM) removal to create Strain 7, and this strain subsequently made competent for TrMDS2 transformation.

Following Pichia DNA transformation, expressed recombinant OVA (rOVA) appeared in culture supernatants of transformants as three distinct species following a 96 h timecourse in methanol-containing media; unglycosylated and mono- and diglycosylated that migrate together as a triplet on SDS-PAGE (see “Input” FIG. 11). To further characterize the OVA expressed by Pichia, supernatants were treated in vitro with commercially available endoglycosidases, EndoH (EH; New England Biolabs) and PNGase (PF; New England Biolabs) using both “native” (N) and “denaturing” (D) protocols for each, as described by the manufacturer (https://www.neb.com/protocols/2012/10/18/endo-hf-protocol; https://www.nebcom/protocols/2014/07/31/pngase-f-protocol). Treatment using either of the endoglycosidases leads to the band-shifted pattern of unglycosylated OVA. The black arrow indicates PNGase F added to the reaction and the grey arrow on the gel indicates the Endo H added to the reaction; the bands appearing above the grey and black arrows are the deglycosylated OVA protein.

An OVA-expressing Pichia strain (Strain 7; described above) was transformed with the Methanol-inducible-TrMDS2 construct (see Example 7). OVA⁺/TrMDS2⁺ transformants were subjected to 10% SDS-PAGE to visualize band-shifting patterns. Shown in FIG. 12, below, is a molecular weight (MW) ladder (lane 1, far left). Lanes labelled “C” contain rOVA produced by the parental OVA-expressing strain (no TrMDS2). Lanes A9, D10, F5, G5, G7, G10, H1 and H2 are from OVA strains transformed with the methanol inducible-TrMDS2 construct. These results suggest that TrMDS2 is capable of removing approximately 1.5-2.5 kDa in carbohydrate from each glycan chain on the Pichia-expressed rOVA.

Transformants were verified by PCR for the presence of TrMDS2 (see Example 7). Transformants A9, D10, F5, G5, G7, G10, H1 and H2 (all shown in the band-shifting gel above) were TrMDS2 positive transformants.

### Example 9: Tr MDS1 Testing

Two different codon-biased TrMDS1 constructs were transformed into a strain expressing Gallus gallus OVD (GgOVD). For expression, the TrMDS1 was placed behind several inducible and constitutive promoters. Construct 1 was engineered for expression of a non-Pichia codon biased (NCO) TrMDS1 cDNA behind the constitutive promoter, construct 2 was engineered for expression of a Pichia codon-optimized (CO) TrMDS1 cDNA behind the constitutive GAP1 promoter, construct 3 was engineered for expression of a Pichia codon-optimized TrMDS1 cDNA behind a methanol-inducible promoter, construct 4 was engineered for expression of a Pichia codon-optimized TrMDS1 cDNA behind a methanol-inducible promoter, construct 5 was engineered for expression of non-Pichia codon-optimized TrMDS1 cDNA behind a methanol-inducible promoter and construct 6 was engineered for expression of a non-Pichia codon-optimized TrMDS1 cDNA behind a methanol-inducible promoter.

Following a timecourse under methanol induction, supernatants were analyzed for GgOVD band shifts. Despite efforts to express these many versions of MDS1, bandshift analysis indicated that the MDS1 was unable to deglycosylate GgOVD. This was in contrast to the new mannosidases exemplified above, MDS2 and the Gallus mannosidase.

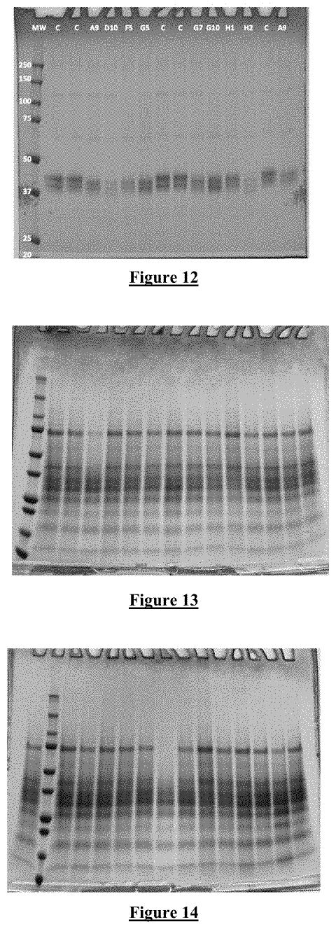

Bandshift gels showing the lack of deglycosylation activity of MDS1 on GgOVD are shown in FIG. 13. Gel 1 (left to right): Molecular weight ladder, Construct 2 GAP-CO_TrMDS1 transformants 1-8, GgOVD strain alone (no mannosidase expression), Construct 1 constitutive-NCO_TrMDS1 transorformant 1, Construct 3 methanol-inducible-TrMDS1 transformants 1 and 2, GgOVD strain alone (no mannosidase expression), Construct 3 transorformant 3.

FIG. 14: Gel 2 (left to right): GgOVD strain alone (no mannosidase expression), Molecular weight ladder, Construct 4 methanol inducible-CO_TrMDS1 transformants 1-8, GgOVD strain alone (no mannosidase expression), Construct 5 methanol inducible-CO_TrMDS1 transformants 1-4.

In total, 240 separate transformants of MDS1 constructs were screened for the ability to deglycosylate GgOVD and none had activity.

### Example 10: Comparison of OVD Glycosylation Patterns

Dry powders consisting of protein samples from Pichia fermentations and from a commercially available source of native chicken ovomucoid were analyzed for total crude protein using a standard combustion method. In this method, total crude protein is calculated from the nitrogen content of the feed material, based on sample type and presented as Percent Protein for the powder in Table 4. The protein factor applied to the nitrogen result is 6.25. The method has a detection limit of 0.1% protein (dry basis). MDS2 (Seq 7) was co-expressed in a Pichia cell along with chicken OVD and the resulting recombinant OVD (rOVD) was purified from the fermentation supernatant using standard protein chromatography methods. Non-protein contaminants were removed from the resulting protein solution using membrane filtration. The purified protein solution was dried to powder using lyophilization. The protein powder was then sent for total crude protein analysis. rOVD powder produced without any MDS2 function had 74% protein on average but that went up to 85% protein when MDS2 was co-expressed. The 85% MDS2-processed material was also a higher % protein relative to the native chicken OVD sample OVD, due to the function of MDS2 removing carbohydrate on the protein.

TABLE 4
Protein content of OVD samples

| Sample type | Strain | N (Total) | % Protein |
|---|---|---|---|
| rOVD with MDS2 | Strain 2 | 13.7 | 85.625 |
| rOVD no | Strain 1 | Not | 74 |
| deglycosylation | | available | |
| Native OVD repeat 1 | — | 12.35 | 77.1875 |
| Native OVD repeat 2 | — | 12.44 | 77.75 |

TABLE 5
Sequences

| Protein | SEQ ID NO | Sequence |
|---|---|---|
| MDS1 | SEQ ID NO: 1 | MRFPSIFTAVLFAASSALAAPVNTTTEDETAQIPAEAVIGYSDLEGDFDVAVLPFSNSTNN |
| | | GLLFINTTIASIAAKEEGVSLEKREAEAATKRGSPNPTRAAAVKAAFQTSWNAYHHFAFP |
| | | HDDLHPVSNSFDDERNGWGSSAIDGLDTAILMGDADIVNTILQYVPQINFTTTAVANQGS |
| | | SVFETNIRYLGGLLSAYDLLRGPFSSLATNQTLVNSLLRQAQTLANGLKVAFTTPSGVPD |
| | | PTVFFNPTVRRSGASSNNVAEIGSLVLEWTRLSDLTGNPQYAQLAQKGESYLLNRKGSPE |
| | | AWPGLIGTFVSTSNGTFQDSSGSWSGLMDSFYEYLIKMYLYDPVAFAHYKDRWVLGAD |
| | | STIGHLGSHPSTRKDLTFLSSYNGQSTSPNSGHLASFGGGNFILGGILLNEQKYIDFGIKLA |
| | | SSYFGTYTQTASGIGPEGFAWVDSVTGAGGSPPSSQSGFYSSAGFWVTAPYYILRPETLES |
| | | LYYAYRVTGDSKWQDLAWEALSAIEDACRAGSAYSSINDVTQANGGGASDDMESFWF |
| | | AEALKYAYLIFAEESDVQVQATGGNKFVFNTEAHPFSIRSSSRRGGHLA* |
| XP_417735.4 | SEQ ID NO: 2 | MVLPRKLPGMPGWPAALGLRLPQKFLFLLFLSGLLTLCFGALFLLPDSSRFKRLFLPRRA |
| PREDICTED: | | TSSSSSSSSSSTRDTELPRSPPAAAEPRHASPAAPRRLREKLRARNAAPAAHTAPASRPQG |
| mannosyl- | | PDGERPAEVGTGAPRESRAPFHFDYERFRQSLRHPVRGGRPDQDPDTRARKMKIKEMM |
| oligosaccharide | | KFAWDNYKQYALGKNELRPLTKNGHIGNMFGGLRGATVVDALDTLYIMELEEEFQEAK |
| 1,2-alpha- | | TWVEKSFDLNVNGEASLFEVNIRYIGGLLAAYYLTGEEVFKSKALELGEKLLPAFNTPTG |
| mannosidase IC | | IPRGVINLGSGMSWSWGWASAGSSILAEFGTLHLEFLHLSELSGNPVFAEKVLNIRKVLK |
| [Gallus gallus] | | RVEKPQGLYPNFLSPVTGNWVQHHVSIGGLGDSFYEYLIKSWLMSDKKDSEAKKMYDD |
| | | ALEAIEKHLVKKSAGGLTYIAEWRGGILDHKMGHLACFSGGMIALGAEHGGEERKQHY |
| | | MDLAAEITNTCHESYARSDTKLGPEAFRFDAGTEAMATRLSERYYILRPEVVESYVYMW |
| | | RLTHDVKYRQWGWEVVKALEKHCRVEAGFSGIRDVYTTVPTHDNMQQSFFLAETLKY |
| | | LYLLFCEDDVLSLDDWVFNTEAHPLPVNHSNFKAKASVQ* |
| no5ManI | SEQ ID NO: 3 | MRCSLFLRLHYESYFWTTLPTNYPPKQIRPLPTTSPLKFPKIQAASPSELPEALKTRLQRQT |
| | | AVKDVFSKCWASYKRHAWKADELAPVSGGQKNPFGGWAATLVDSLDTLYLMDMKPE |
| | | FDEAVAAAASIDFTKTDLDEVNVFETTIRYLGGFLSAYDLSADARLLSKAVEVGEMLYH |
| | | AFDTPNRMPITRWAIHAAMAGKKQVAPAGLLVAEIGSLSMEFTRLSMLTRDPKWFDAV |
| | | QRITEGMAAQQNATALPGLWPLVVSAQDEIYSVGDTFTLGAMADSVYEYLPKMSALTG |
| | | GQLPVYREMYEAAMATALKHNLFRPMTPSNQDILVAGTVKADGGVKTTLEPQGQHLV |
| | | CFLGGLLTLGGKLFGRQQDLDAARRLVDGCVWTYKALPRGIMPETFFMLPCPSSTCAW |
| | | DEASWKRGVLARAAKDAADKASDDDDADAIISRDRLPKGFTSIPDRRYILRPEAIESVFV |
| | | SYRATAEPSLMESAWDMFTAINATTSTRLANSAYWDVTRPMGEDPGMADSMESFWMG |
| | | ETLKYFYLVFAAWDDVSLDEWVFNTEAHPFRRLLP* |
| no4ManI | SEQ ID NO: 4 | MLNQLQGRVPRRYIALVAFAFFVAFLLWSGYDFVPRTATVGRFKYVPSSYDWSKAKVY |
| | | YPVKDMKTLPQGTPVTFPRLQLRNQSEAQDDTTKARKQAVKDAFVKSWEAYKTYAWT |
| | | KDQLQPLSLSGKETFSGWSAQLVDALDTLWIMDLKDDFFLAVKEVAVIDWSKTKDNKV |
| | | INLFEVTIRYLGGLIAAYDLSQEPVLRAKAIELGDTLYATFDTPNRLPSHWLDYSKAKKG |
| | | TQRADDSMSGAAGGTLCMEFTRLSQITGDPKYYDATERIKQFFYRFQNETTLPGMWFV |
| | | MMNYREETMVESRYSMGGSADSLYEYLVKMPALLGGLDPQYPEMAIRALDTARDNLL |
| | | FRPMTEKGDNILALGNALVDHGNVQRTTEMQHLTCFAGGMYAMAGKLFKRDDYVDLG |
| | | SRISSGCVWAYDSFPSGIMPESADMAACAKLDGPCPYDEVKAPVDPDGRRPHGFIHVKS |
| | | RHYLLRPEAIESVFYMWRITGDQVWRDTAWRMWENIVREAETEHAFAIVEDVTRTASK |
| | | LTNNTYLLQTFWLAETLKYFYLIFDDESAIDLDKWVFNTEAHPFKRPAV* |
| no3ManI | SEQ ID NO: 5 | MVMLVAIALAWLGCSLLRPVDAMRADYLAQLRQETVDMFYHGYSNYMEHAFPEDELR |
| | | PISCTPLTRDRDNPGRISLNDALGNYSLTLIDSLSTLAILAGGPQNGPYTGPQALSDFQDG |
| | | VAEFVRHYGDGRSGPSGAGIRARGFDLDSKVQVFETVIRGVGGLLSAHLFAIGELPITGY |
| | | VPRPEGVAGDDPLELAPIPWPNGFRYDGQLLRLALDLSERLLPAFYTPTGIPYPRVNLRSG |
| | | IPFYVNSPLHQNLGEAVEEQSGRPEITETCSAGAGSLVLEFTVLSRLTGDARFEQAAKRAF |
| | | WEVWHRRSEIGLIGNGIDAERGLWIGPHAGIGAGMDSFFEYALKSHILLSGLGMPNASTS |
| | | RRQSTTSWLDPNSLHPPLPPEMHTSDAFLQAWHQAHASVKRYLYTDRSHFPYYSNNHR |
| | | ATGQPYAMWIDSLGAFYPGLLALAGEVEEAIEANLVYTALWTRYSALPERWSVREGNV |
| | | EAGIGWWPGRPEFIESTYHIYRATRDPWYLHVGEMVLRDIRRRCYAECGWAGLQDVQT |
| | | GEKQDRMESFFLGETAKYMYLLFDPDHPLNKLDAAYVFTTEGHPLIIPKSKRGSGSHNR |
| | | QDRARKAKKSRDVAVYTYYDESFTNSCPAPRPPSEHHLIGSATAARPDLFSVSRFTDLYR |
| | | TPNVHGPLEKVEMRDKKKGRVVRYRATSNHTIFPWTLPPAMLPENGTCAAPPERIISLIEF |
| | | PANDITSGITSRFGNHLSWQTHLGPTVNILEGLRLQLEQVSDPATGEDKWRITHIGNTQLG |
| | | RHETVFFHAEHVRHLKDEVFSCRRRRDAVEIELLVDKPSDTNNNNTLASSDDDVVVDAK |
| | | AEEQDGMLADDDGDTLNAETLSSNSLFQSLLRAVSSVFEPVYTAIPESDPSAGTAKVYSF |
| | | DAYTSTGPGAYPMPSLSDTPIPGNPFYNFRNPASNFPWSTVFLAGQACEGPLPASAPREHQ |
| | | VTVMLRGGCSFSRKLDNIPSFSPHDRALQLVVVLDEPPPPPPPPPANDRRDVTRPLLDTEQ |
| | | TTPKGMKRLHGIPMVLVRAARGDYELFGHAIGVGMRRKYRVESQGLVVENAVVL* |
| no2ManI | SEQ ID NO: 6 | MRFPSSSVLALGLIGPALAYPKPGATKRGSPNPTRAAAVKAAFQTSWNAYHHFAFPHDD |
| | | LHPVSNSFDDERNGWGSSAIDGLDTAILMGDADIVNTILQYVPQINFTTTAVANQGISVFE |
| | | TNIRYLGGLLSAYDLLRGPFSSLATNQTLVNSLLRQAQTLANGLKVAFTTPSGVPDPTVF |
| | | FNPTVRRSGASSNNVAEIGSLVLEWTRLSDLTGNPQYAQLAQKGESYLLNPKGSPEAWP |
| | | GLIGTFVSTSNGTFQDSSGSWSGLMDSFYEYLIKMYLYDPVAFAHYKDRWVLAADSTIA |
| | | HLASHPSTRKDLTFLSSYNGQSTSPNSGHLASFAGGNFILGGILLNEQKYIDFGIKLASSYF |
| | | ATYNQTASGIGPEGFAWVDSVTGAGGSPPSSQSGFYSSAGFWVTAPYYILRPETLESLYY |
| | | AYRVTGDSKWQDLAWEAFSAIEDACRAGSAYSSINDVTQANGGGASDDMESFWFAEAL |
| | | KYAYLIFAEESDVQVQANGGNKFVFNTEAHPFSIRSSSRRGGHLA* |
| no1ManI | SEQ ID NO: 7 | MARRRYRLFMICAAVILFLLYRVSQNTWDDSAHYATLRHPPASNPPAAGGESPLKPAAK |
| | | PEHEHEHENGYAPESKPKPQSEPKPESKPAPEHAAGGQKSQGKPSYEDDEETGKNPPKSA |
| | | VIPSDTRLPPDNKVHWRPVKEHFPVPSESVISLPTGKPLKVPRVQHEFGVESPEAKSRRVA |
| | | RQERVGKEIERAWSGYKKFAWMHDELSPVSAKHRDPFCGWAATLVDSLDTLWIAGLKE |
| | | QFDEAARAVEQIDFTTTPRNNIPVFETTIRYLGGLLGAFDVSGGHDGGYPMLLTKAVELA |
| | | EILMGIFDTPNRMPILYYQWQPEYASQPHRAGSVGIAELGTLSMEFTRLAQLTSQYKYYD |
| | | AVDRITDALIELQKQGTSIPGLFPENDASGCNHTATALRSSLSEAAQKQMDEDLSNKPE |
| | | NYRPGKNSKADPQTVEKQPAKKQNEPVEKAKQVPTQQTAKRGKPPFGANGFTANWDC |
| | | VPQGLVVGGYGFQQYHMGGGQDSAYEYFPKEYLLLGGLESKYQKLYVDAVEAINEWL |
| | | LYRPMTDGDWDILFPAKVSTAGNPSQDLVATFEVTHLTCFIGGMYGLGGKIFGREKDLE |
| | | TAKRLTDGCVWAYQSTVSGIMPEGSQVLACPTLEKCDFNETLWWEKLDPAKDWRDKQ |
| | | YADDKDKATVGEALKETANSHDAAGGSKAVHKRAAVPLPKPGADDDVGSELPQSLKD |
| | | KIGFKNGEQKKPTGSSVGIQRDPDAPVDSVLEAHRLPPQEPEEQQVILPDKPQTHEEFVK |
| | | QRIAEMGFAPGVVHIQSRQYILRPEAIESVWYMYRITGDPIWMEKGWKMFEATIRATRTE |
| | | INSAIDDVNSEEPGLKDEMESFWLAETLKYYYLLFSEPSVISLDEWVLNTEAHPFKRPG |
| | | GSYIGHSI* |
| patMannI | SEQ ID NO: 8 | MRFPSSSVLALGLIGPALAYPKPGATKRGSPNPTRAAAVKAAFQTSWNAYHHFAFPHDD |
| | | LHPVSNSFDDERNGWGSSAIDGLDTAILMGDADIVNTILQYVPQINFTTTAVANQGISVFE |
| | | TNIRYLGGLLSAYDLLRGPFSSLATNQTLVNSLLRQAQTLANGLKVAFTTPSGVPDPTVF |
| | | FNPTVRRSGASSNNVAEIGSLVLEWTRLSDLTGNPQYAQLAQKGESYLLNPKGSPEAWP |
| | | GLIGTFVSTSNGTFQDSSGSWSGLMDSFYEYLIKMYLYDPVAFAHYKDRWVLAADSTIA |
| | | HLASHPSTRKDLTFLSSYNGQSTSPNSGHLASFAGGNFILGGILLNEQKYIDFGIKLASSYF |
| | | ATYNQTASGIGPEGFAWVDSVTGAGGSPPSSQSGFYSSAGFWVTAPYYILRPETLESLYY |
| | | AYRVTGDSKWQDLAWEAFSAIEDACRAGSAYSSINDVTQANGGGASDDMESFWFAEAL |
| | | KYAYLIFAEESDVQVQANGGNKFVFNTEAHPFSIRSSSRRGGHLA* |
| AAF34579.1 1,2-a- | SEQ ID NO: 9 | MRFPSSSVLALGLIGPALAYPKPGATKRGSPNPTRAAAVKAAFQTSWNAYHHFAFPHDD |
| D-mannosidase | | LHPVSNSFDDERNGWGSSAIDGLDTAILMGDADIVNTILQYVPQINFTTTAVANQGSSVF |
| [Trichoderma | | ETNIRYLGGLLSAYDLLRGPFSSLATNQTLVNSLLRQAQTLANGLKVAFTTPSGVPDPTV |
| reesei] | | FFNPTVRRSGASSNNVAEIGSLVLEWTRLSDLTGNPQYAQLAQKGESYLLNPKGSPEAW |
| | | PGLIGTFVSTSNGTFQDSSGSWSGLMDSFYEYLIKMYLYDPVAFAHYKDRWVLGADSTI |
| | | GHLGSHPSTRKDLTFLSSYNGQSTSPNSGHLASFGGGNFILGGILLNEQKYIDFGIKLASSY |
| | | FGTYTQTASGIGPEGFAWVDSVTGAGGSPPSSQSGFYSSAGFWVTAPYYILRPETLESLY |
| | | YAYRVTGDSKWQDLAWEALSAIEDACRAGSAYSSINDVTQANGGGASDDMESFWFAE |
| | | ALKYAYLIFAEESDVQVQATGGNKFVFNTEAHPFSIRSSSRRGGHLA* |
| Hypacrea MDS1 | SEQ ID NO: 10 | MRFPSIFTAVLFAASSALAAPVNTTTEDETAQIPAEAVIGYSDLEGDFDVAVLPFSNSTNN |
| | | GLLFINTTIASIAAKEEGVSLEKREAEAATKRGSPNPTRAAAVKAAFQTSWNAYHHFAFP |
| | | HDDLHPVSNSFDDERNGWGSSAIDGLDTAILMGDADIVNTILQYVPQINFTTTAVANQGS |
| | | SVFETNIRYLGGLLSAYDLLRGPFSSLATNQTLVNSLLRQAQTLANGLKVAFTTPSGVPD |
| | | PTVFFNPTVRRSGASSNNVAEIGSLVLEWTRLSDLTGNPQYAQLAQKGESYLLNPKGSPE |
| | | AWPGLIGTFVSTSNGTFQDSSGSWSGLMDSFYEYLIKMYLYDPVAFAHYKDRWVLGAD |
| | | STIGHLGSHPSTRKDLTFLSSYNGQSTSPNSGHLASFGGGNFILGGILLNEQKYIDFGIKLA |
| | | SSYFGTYTQTASGIGPEGFAWVDSVTGAGGSPPSSQSGFYSSAGFWVTAPYYILRPETLES |
| | | LYYAYRVTGDSKWQDLAWEALSAIEDACRAGSAYSSINDVTQANGGGASDDMESFWF |
| | | AEALKYAYLIFAEESDVQVQATGGNKFVFNTEAHPFSIRSSSRRGGHLA* |
| α-ovomucin | SEQ ID NO: 11 | KEPVQIVQVSTVGRSECTTWGNFHFHTFDHVKFTFPGTCTYVFASHCNDSYQDFNIKIRR |
| | | SDKNSHLIYFTVTTDGVILEVKETGITVNGNQIPLPFSLKSILIEDTCAYFQVTSKLGLTLK |
| | | WNWADTLLLDLEETYKEKICGLCGNYDGNKKNDLILDGYKMHPRQFGNFHKVEDPSEK |
| | | CPDVRPDDHTGRHPTEDDNRCSKYKKMCKKLLSRFGNCPKVVAFDDYVATCTEDMCN |
| | | CVVNSSHSDLVSSCICSTLNQYSRDCVLSKGDPGEWRTKELCYQECPSNMEYMECGNSC |
| | | ADTCADPERSKICKAPCTDGCFCPPGTILDDLGGKKCVPRDSCPCMFQGKVYSSGGTYST |
| | | PCQNCTCKGGHWSCTSLPCSGSCSIDGGFHITTFDNKKFNFHGNCHYVLAKNTDDTFVVI |
| | | GEIIQCGTSKT*MTCLKNVLVTLGRTTIKICSCGSIYMNNFIVKLPVSKDGITIFRPSTFFIKI |
| | | LSSTGVQIRVQMKPVMQLSITVDHSYQNRTSGLCGNFNNIQTDDFRTATGAVEDSAAAF |
| | | GNSWKTRASCFDVEDSFEDPCSNSVDKEKFAQHVVCALLSNISSTFAACHSVVDPSVYIKR |
| | | CMYDTCNAEKSEVALCSVLSTYSRDCAAAGMTLKGWRQGICDPSEECPETMVYNYSVK |
| | | YCNQSCRSLDEPDPLCKVQIAPMEGCGCPEGTYLNDEEECVTPDDCPCYYKGKIVQPGN |
| | | SFQEDKLLCKCIQGRLDCIGETVLVKDCPAPMYYFNCSSAGPGAIGSECQKSCKTQDMH |
| | | CYVTECVSGCMCPDGLVLDGSGGCIPKDQCPCVHGGHFYKPGETIRVDCNTCTCNKRQ |
| | | WNCTDSPCKGTCTVYGNGHYMSFDGEKFDFLGDCDYILAQDFCPNNMDAGTFRIVIQN |
| | | NACGKSLSICSLKITLIFESSEIRLLEGRIQEIATDPGAEKNYKVDLRGGYIVIETTQGMSFM |
| | | WDQKTTVVVHVTPSFQGKVCGLCGDFDGRSRNDFTTRGQSVEMSIQEFGNSWKITSTCS |
| | | NINMTDLCADQPFKSALGQKHCSIIKSSVFEACHSKVNPIPYYESCVSDFCGCDSVGDCEC |
| | | FCTSVAAYARSCSTAGVCINWRTPAICPVFCDYYNPPDKHEWFYKPCGAPCLKTCRNPQ |
| | | GKCGNILYSLEGCYPECSPDKPYFDEERRECVSLPDCTSCNPEEKLCTEDSKDCLCCYNG |
| | | KTYPLNETIYSQTEGTKCGNAFCGPNGMIIETFIPCSTLSVPAQEQLMQPVTSAPLLSTEAT |
| | | PCFCTDNGQLIQMGENVSLPMNISGHCAYSICNASCQIELIWAECKVVQTEALETCEPNSE |
| | | ACPPTAAPNATSLVPATALAPMSDCLGLIPPRKFNESWDFGNCQIATCLGEENNIKLSSIT |
| | | CPPQQLKLCVNGFPFMKHHDETGCCEVFECQCICSGWGNEHYVTFDGTYYHFKENCTY |
| | | VLVELIQPSSEKFWIHIDNYYCGAADGAICSMSLLIFHSNSLVILTQAKEHGKGTNLVLFN |
| | | DKKVVPDISKNGIRITSSGLYIIVEIPELEVYVSYSRLAFYIKLPFGKYYNNTMGLCGTCTN |
| | | QKSDDARKRNGEVTDSFKEMALDWKAPVSTNRYCNPGISEPVKIENYQHCEPSELCKII |
| | | WNLTECHRVVPPQPYYEACVASRCSQQHPSTECQSMQTYAALCGLHGICVDWRGQTNG |
| | | QCEATCARDQVYKPCGEAKRNTCFSREVIVDTLLSRNNTPVFVEGCYCPDGNILLNEHD |
| | | GICVSVCGCTAQDGSVKKPREAWEHDCQYCTCDEETLNISCFPRPCAKSPPINCTKEGFV |
| | | RKIKPRLDDPCCTETVCECDIKTCIINKTACDLGFQPVVAISEDGCCPIFSCIPKGVCVSEG |
| | | VEFKPGAVVPKSSCEDCVCTDEQDAVTGTNRIQCVPVKCQTTCQQGFRYVEKEGQCCSQ |
| | | CQQVACVANFPFGSVTIEVGKSYKAPYDNCTQYTCTESGGQFSLTSTVKVCLPFEESNCV |
| | | PGTVDVTSDGCCKTCIDLPHKCKRSMKEQYIVHKHCKSAAPVPVPFCEGTCSTYSVYSFE |
| | | NNEMEHKCICCHEKKSHVEKVELVCSEHKTLKFSYVHVDECGCVETKCPMRRT* |
| Ovomucoid | SEQ ID NO: 12 | AEVDCSRFPNATDKEGKDVLVCNKDLRPICGTDGVTYTNDCLLCAYSIEFGTNISKEHDG |
| (canonical) | | ECKETVPMNCSSYANTTSEDGKVMVLCNRAFNPVCGTDGVTYDNECLLCAHKVEQGAS |
| | | VDKRHDGGCRKELAAVSVDCSEYPKPDCTAEDRPLCGSDNKTYGNKCNFCNAVVESNG |
| | | TLTLSHFGKC* |
| Ovomucoid | SEQ ID NO: 13 | AEVDCSRFPNATDMEGKDVLVCNKDLRPICGTDGVTYTNDCLLCAYSVEFGTNISKEHD |
| | | GECKETVPMNCSSYANTTSEDGKVMVLCNRAFNPVCGTDGVTYDNECLLCAHKVEQG |
| | | ASVDKRHDGGCRKELAAVSVDCSEYPKPDCTAEDRPLCGSDNKTYGNKCNFCNAVVES |
| | | NGTLTLSHFGKC* |
| Ovomucoid | SEQ ID NO: 14 | AEVDCSRFPNATDMEGKDVLVCNKDLRPICGTDGVTYTNDCLLCAYSVEFGTNISKEHD |
| G162MF167A | | GECKETVPMNCSSYANTTSEDGKVMVLCNRAFNPVCGTDGVTYDNECLLCAHKVEQG |
| | | ASVDKRHDGGCRKELAAVSVDCSEYPKPDCTAEDRPLCGSDNKTYMNKCNACNAVVE |
| | | SNGTLTLSHFGKC* |
| Ovoglobulin G2 | SEQ ID NO: 15 | TRAPDCGGILTPLGLSYLAEVSKPHAEVVLRQDLMAQRASDLFLGSMEPSRNRITSVKVA |
| | | DLWLSVIPEAGLRLGIEVELRIAPLHAVPMPVRISIRADLHVDMGPDGNLQLLTSACRPTV |
| | | QAQSTREAESKSSRSILDKVVDVDKLCLDVSKLLLFPNEQLMSLTALFPVTPNCQLQYLP |
| | | LAAPVFSKQGIALSLQTTFQVAGAVVPVPVSPVPFSMPELASTSTSHLILALSEHFYTSLYF |
| | | TLERAGAFNMTIPSMLTTATLAQKITQVGSLYHEDLPITLSAALRSSPRVVLEEGRAALKL |
| | | FLTVHIGAGSPDFQSFLSVSADVTAGLQLSVSDTRMMISTAVIEDAELSLAASNVGLVRA |
| | | ALLEELFLAPVCQQVPAWMDDVLREGVHLPHLSHFTYTDVNVVVHKDYVLVPCKLKLR |
| | | STMA* |
| Ovoglobulin G3 | SEQ ID NO: 16 | MDSISVTNAKFCFDVFNEMKVHHVNENILYCPLSILTALAMVYLGARGNTESQMKKVL |
| | | HFDSITGAGSTTDSQCGSSEYVHNLFKELLSEITRPNATYSLEIADKLYVDKTFSVLPEYLS |
| | | CARKFYTGGVEEVNFKTAAEEARQLINSWVEKETNGQIKDLLVSSSIDFGTTMVFINTIYF |
| | | KGIWKIAFNTEDTREMPFSMTKEESKPVQMMCMNNSFNVATLPAEKMKILELPYASGDL |
| | | SMLVLLPDEVSGLERIEKTINFDKLREWTSTNAMAKKSMKVYLPRMKIEEKYNLTSILM |
| | | ALGMTDLFSRSANLTGISSVDNLMISDAVHGVFMEVNEEGTEATGSTGAIGNIKHSLELE |
| | | EFRADHPFLFFIRYNPTNAILFFGRYWSP* |
| β-ovomucin | SEQ ID NO: 17 | CSTWGGGHFSTFDKYQYDFTGTCNYIFATVCDESSPDFNIQFRRGLDKKIARIIIELGPSVII |
| | | VEKDSISVRSVGVIKLPYASNGIQIAPYGRSVRLVAKLMEMELVVMWNNEDYLMVLTE |
| | | KKYMGKTCGMCGNYDGYELNDFVSEGKLLDTYKFAALQKMDDPSEICLSEEISIPAIPH |
| | | KKYAVICSQLLNLVSPTCSVPKDGFVTRCQLDMQDCSEPGQKNCTCSTLSEYSRQCAMS |
| | | HQVVFNWRTENFCSVGKCSANQIYEECGSPCIKTCSNPEYSCSSHCTYGCFCPEGTVLDD |
| | | ISKNRTCVHLEQCPCTLNGETYAPGDTMKAACRTCKCTMGQWNCKELPCPGRCSLEGG |
| | | SFVTTFDSRSYRFHGVCTYILMKSSSLPHNGTLMAIYEKSGYSHSETSLSAIIYLSTKDKIVI |
| | | SQNELLTDDDELKRLPYKSGDITIFKQSSMFIQMHTEFGLELVVQTSPVFQAYVKVSAQF |
| | | QGRTLGLCGNYNGDTTDDFMTSMDITEGTASLFVDSWRAGNCLPAMERETDPCALSQL |
| | | NKISAETHCSILTKKGTVFETCHAVVNPTPFYKRCVYQACNYEETFPYICSALGSYARTCS |
| | | SMGLILENWRNSMDNCTITCTGNQTFSYNTQACERTCLSLSNPTLECHPTDIPIEGCNCPK |
| | | GMYLNHKNECVRKSHCPCYLEDRKYILPDQSTMTGGITCYCVNGRLSCTGKLQNPAESC |
| | | KAPKKYISCSDSLENKYGATCAPTCQMLATGIECIPTKCESGCVCADGLYENLDGRCVPP |
| | | EECPCEYGGLSYGKGEQIQTECEICTCRKGKWKCVQKSRCSSTCNLYGEGHITTFDGQRF |
| | | VFDGNCEYILAMDGCNVNRPLSSFKIVTENVICGKSGVTCSRSISIYLGNLTIILRDETYSIS |
| | | GKNLQVKYNVKKNALHLMFDIIIPGKYNMTLIWNKHMNFFIKISRETQETICGLCGNYNG |
| | | NMKDDFETRSKYVASNELEFVNSWKENPLCGDVYFVVDPCSKNPYRKAWAEKTCSIINS |
| | | QVFSACHNKVNRMPYYEACVRDSCGCDIGGDCECMCDAIAVYAMACLDKGICIDWRTP |
| | | EFCPVYCEYYNSHRKTGSGGAYSYGSSVNCTWHYRPCNCPNQYYKYVNIEGCYNCSHD |
| | | EYFDYEKEKCMPCAMQPTSVTLPTATQPTSPSTSSASTVLTETTNPPV* |
| Lysozyme | SEQ ID NO: 18 | KVFGRCELAAAMKRHGLDNYRGYSLGNWVCAAKFESNFNTQATNRNTDGSTDYGILQI |
| | | NSRWWCNDGRTPGSRNLCNIPCSALLSSDITASVNCAKKIVSDGNGMNAWVAWRNRCK |
| | | GTDVQAWIRGCRL* |
| Lysozyme | SEQ ID NO: 19 | KVFGRCELAAAMKRHGLDNYRGYSLGNWVCVAKFESNFNTQATNRNTDGSTDYGILQI |
| | | NSRWWCNDGRTPGSRNLCNIPCSALLSSDITASVNCAKKIVSDGNGMSAWVAWRNRCK |
| | | GTDVQAWIRGCRL* |
| Lysozyme C | SEQ ID NO: 20 | KVFERCELARTLKRLGMDGYRGISLANWMCLAKWESGYNTRATNYNAGDRSTDYGIF |
| (Human) | | QINSRYWCNDGKTPGAVNACHLSCSALLQDNIADAVACAKRVVRDPQGIRAWVAWRN |
| | | RCQNRDVRQYVQGCGV* |
| Lysozyme C (Bos | SEQ ID NO: 21 | KVFERCELARTLKKLGLDGYKGVSLANWLCLTKWESSYNTKATNYNPSSESTDYGIFQI |
| taurus) | | NSKWWCNDGKTPNAVDGCHVSCRELMENDIAKAVACAKHIVSEQGITAWVAWKSHCR |
| | | DHDVSSYVEGCTL* |
| Ovoinhibitor | SEQ ID NO: 22 | IEVNCSLYASGIGKDGTSWVACPRNLKPVCGTDGSTYSNECGICLYNREHGANVEKEYD |
| | | GECRPKHVMIDCSPYLQVVRDGNTMVACPRILKPVCGSDSFTYDNECGICAYNAEHHTN |
| | | ISKLHDGECKLEIGSVDCSKYPSTVSKDGRTLVACPRILSPVCGTDGFTYDNECGICAHNA |
| | | EQRTHVSKKHDGKCRQEIPEIDCDQYPTRKTTGGKLLVRCPRILLPVCGTDGFTYDNECG |
| | | ICAHNAQHGTEVKKSHDGRCKERSTPLDCTQYLSNTQNGEAITACPFILQEVCGTDGVTY |
| | | SNDCSLCAHNIELGTSVAKKHDGRCREEVPELDCSKYKTSTLKDGRQVVACTMIYDPVC |
| | | ATNGVTYASECTLCAHNLEQRTNLGKRKNGRCEEDITKEHCREFQKVSPICTMEYVPHC |
| | | GSDGVTYSNRCFFCNAYVQSNRTLNLVSMAAC* |
| Cystatin | SEQ ID NO: 23 | MAGARGCVVLLAAALMLVGAVLGSEDRSRLLGAPVPVDENDEGLQRALQFAMAEYNR |
| | | ASNDKYSSRVVRVISAKRQLVSGIKYILQVEIGRTTCPKSSGDLQSCEFHDEPEMAKYTTC |
| | | TFVVYSIPWLNQIKLLESKCQ* |
| Ovalbumin related | SEQ ID NO: 24 | MFFYNTDFRMGSISAANAEFCFDVFNELKVQHTNENILYSPLSIIVALAMVYMGARGNTE |
| protein X | | YQMEKALHFDSIAGLGGSTQTKVQKPKCGKSVNIHLLLFKELLSDITASKANYSLRIANRL |
| | | YAEKSRPILPIYLKCVKKLYRAGLETVNFKTASDQARQLINSWVEKQTEGQIKDLLVSSS |
| | | TDLDTTLVLVNAIYFKGMWKTAFNAEDTREMPFHVTKEESKPVQMMCMNNSFNVATL |
| | | PAEKMKILELPFASGDLSMLVLLPDEVSGLERIEKTINFEKLTEWTNPNTMEKRRVKVYL |
| | | PQMKIEEKYNLTSVLMALGMTDLFIPSANLTGISSAESLKISQAVHGAFMELSEDGIEMA |
| | | GSTGVIEDIKHSPELEQFRADHPFLFLIKHNPTNTIVYFGRYWSP* |
| Ovalbumin related | SEQ ID NO: 25 | MDSISVTNAKFCFDVFNEMKVHHVNENILYCPLSILTALAMVYLGARGNTESQMKKVL |
| protein Y | | HFDSITGAGSTTDSQCGSSEYVHNLFKELLSEITRPNATYSLEIADKLYVDKTFSVLPEYLS |
| | | CARKFYTGGVEEVNFKTAAEEARQLINSWVEKETNGQIKDLLVSSSIDFGTTMVFINTIYF |
| | | KGIWKIAFNTEDTREMPFSMTKEESKPVQMMCMNNSFNVATLPAEKMKILELPYASGDL |
| | | SMLVLLPDEVSGLERIEKTINFDKLREWTSTNAMAKKSMKVYLPRMKIEEKYNLTSILM |
| | | ALGMTDLFSRSANLTGISSVDNLMISDAVHGVFMEVNEEGTEATGSTGAIGNIKHSLELE |
| | | EFRADHPFLFFIRYNPTNAILFFGRYWSP* |
| Ovalbumin | SEQ ID NO: 26 | MGSIGAASMEFCFDVFKELKVHHANENIFYCPIAIMSALAMVYLGAKDSTRTQINKVVR |
| | | FDKLPGFGDSIEAQCGTSVNVHSSLRDILNQITKPNDVYSFSLASRLYAEERYPILPEYLQC |
| | | VKELYRGGLEPINFQTAADQARELINSWVESQTNGIIRNVLQPSSVDSQTAMVLVNAIVF |
| | | KGLWEKAFKDEDTQAMPFRVTEQESKPVQMMYQIGLFRVASMASEKMKILELPFASGT |
| | | MSMLVLLPDEVSGLEQLESIINFEKLTEWTSSNVMEERKIKVYLPRMKMEEKYNLTSVL |
| | | MAMGITDVFSSSANLSGISSAESLKISQAVHAAHAEINEAGREVVGSAEAGVDAASVSEE |
| | | FRADHPFLFCIKHIATNAVLFFGRCVSP* |
| Porcine Lipase | SEQ ID NO: 27 | SEVCFPRLGCFSDDAPWAGIVQRPLKILPWSPKDVDTRFLLYTNQNQNNYQELVADPSTI |
| | | TNSNFRMDRKTRFIIHGFIDKGEEDWLSNICKNLFKVESVNCICVDWKGGSRTGYTQASQ |
| | | NIRIVGAEVAYFVEVLKSSLGYSPSNVHVIGHSLGSHAAGEAGRRTNGTIERITGLDPAEP |
| | | CFQGTPELVRLDPSDAKFVDVIHTDAAPIIPNLGFGMSQTVGHLDFFPNGGKQMPGCQK |
| | | NILSQIVDIDGIWEGTRDFVACNHLRSYKYYADSILNPDGFAGFPCDSYNVFTANKCFPCP |
| | | SEGCPQMGHYADRFPGKTNGVSQVFYLNTGDASNFARWRYKVSVTLSGKKVTGHILVS |
| | | LFGNEGNSRQYEIYKGTLQPDNTHSDEFDSDVEVGDLQKVKFIWYNNNVINPTLPRVGA |
| | | SKITVERNDGKVYDFCSQETVREEVLLTLNPC* |
| Kid Lipase | SEQ ID NO: 28 | GLVAADRITGGKDFRDIESKFALRTPEDTAEDTCHLIPGVTESVANCHFNHSSKTFVVIHG |
| | | WTVTGMYESWVPKLVAALYKREPDSNVIVVDWLSRAQQHYPVSAGYTKLVGQDVAKF |
| | | MNWMADEFNYPLGNVHLLGYSLGAHAAGIAGSLTSKKVNRITGLDPAGPNFEYAEAPS |
| | | RLSPDDADFVDVLHTFTRGSPGRSIGIQKPVGHVDIYPNGGTFQPGCNIGEALRVIAERGL |
| | | GDVDQLVKCSHERSVHLFIDSLLNEENPSKAYRCNSKEAFEKGLCLSCRKNRCNNMGYE |
| | | INKVRAKRSSKMYLKTRSQMPYKVFHYQVKRIFSGTESNTYTNQAFEISLYGTVAESENI |
| | | PFTLPEVSTNKTYSFLLYTEVDIGELLMLKLKWISDSYFSWSNWWSSPGFDIGKIRVKAG |
| | | ETQKKVIFCSREKMSYLQKGKSPVIFVKCHDKSLNRKSG* |
| Porcine | SEQ ID NO: 29 | APKKGVRWCVISTAEYSKCRQWQSKIRRTNPMFCIRRASPTDCIRAIAAKRADAVTLDG |
| Lactoferrin | | GLVFEADQYKLRPVAAEIYGTEENPQTYYYAVAVVKKGFNFQLNQLQGRKSCHTGLGR |
| | | SAGWNIPIGLLRRFLDWAGPPEPLQKAVAKFFSQSCVPCADGNAYPNLCQLCIGKGKDK |
| | | CACSSQEPYFGYSGAFNCLHKGIGDVAFVKESTVFENLPQKADRDKYELLCPDNTRKPV |
| | | EAFRECHLARVPSHAVVARSVNGKENSIWELLYQSQKKFGKSNPQEFQLFGSPGQQKDL |
| | | LFRDATIGFLKIPSKIDSKLYLGLPYLTAIQGLRETAAEVEARQAKVVWCAVGPEELRKC |
| | | RQWSSQSSQNLNCSLASTTEDCIVQVLKGEADAMSLDGGFIYTAGKCGLVPVLAENQKS |
| | | RQSSSSDCVHRPTQGYFAVAVVRKANGGITWNSVRGTKSCHTAVDRTAGWNIPMGLLV |
| | | NQTGSCKFDEFFSQSCAPGSQPGSNLCALCVGNDQGVDKCVPNSNERYYGYTGAFRCLA |
| | | ENAGDVAFVKDVTVLDNTNGQNTEEWARELRSDDFELLCLDGTRKPVTEAQNCHLAV |
| | | APSHAVVSRKEKAAQVEQVLLTEQAQFGRYGKDCPDKFCLFRSETKNLLFNDNTEVLA |
| | | QLQGKTTYEKYLGSEYVTAIANLKQCSVSPLLEACAFMMR* |
| Bovine | SEQ ID NO: 30 | APRKNVRWCTISQPEWFKCRRWQWRMKKLGAPSITCVRRAFALECIRAIAEKKADAVT |
| Lactoferrin | | LDGGMVFEAGRDPYKLRPVAAEIYGTKESPQTHYYAVAVVKKGSNFQLDQLQGRKSCH |
| | | TGLGRSAGWIIPMGILRPYLSWTESLEPLQGAVAKFFSASCVPCIDRQAYPNLCQLCKGE |
| | | GENQCACSSREPYFGYSGAFKCLQDGAGDVAFVKETTVFENLPEKADRDQYELLCLNNS |
| | | RAPVDAFKECHLAQVPSHAVVARSVDGKEDLIWKLLSKAQEKFGKNKSRSFQLFGSPPG |
| | | QRDLLFKDSALGFLRIPSKVDSALYLGSRYLTTLKNLRETAEEVKARYTRVVWCAVGPE |
| | | EQKKCQQWSQQSGQNVTCATASTTDDCIVLVLKGEADALNLDGGYIYTAGKCGLVPVL |
| | | AENRKSSKHSSLDCVLRPTEGYLAVAVVKKANEGLTWNSLKDKKSCHTAVDRTAGWNI |
| | | PMGLIVNQTGSCAFDEFFSQSCAPGADPKSRLCALCAGDDQGLDKCVPNSKEKYYGYTG |
| | | AFRCLAEDVGDVAFVKNDTVWENTNGESTADWAKNLNREDFRLLCLDGTRKPVTEAQ |
| | | SCHLAVAPNHAVVSRSDRAAHVKQVLLHQQALFGKNGKNCPDKFCLFKSETKNLLFND |
| | | NTECLAKLGGRPTYEEYLGTEYVTAIANLKKCSTSPLLEACAFLTR* |
| AOX1 | SEQ ID NO: 31 | GATCTAACATCCAAAGACGAAAGGTTGAATGAAACCTTTTTGCCATCCGACATCCAC |
| | | AGGTCCATTCTCACACATAAGTGCCAAACGCAACAGGAGGGGATACACTAGCAGCA |
| | | GACCGTTGCAAACGCAGGACCTCCACTCCTCTTCTCCTCAACACCCACTTTTGCCATC |
| | | GAAAAACCAGCCCAGTTATTGGGCTTGATTGGAGCTCGCTCATTCCAATTCCTTCTAT |
| | | TAGGCTACTAACACCATGACTTTATTAGCCTGTCTATCCTGGCCCCCCTGGCGAGGTT |
| | | CATGTTTGTTTATTTCCGAATGCAACAAGCTCCGCATTACACCCGAACATCACTCCAG |
| | | ATGAGGGCTTTCTGAGTGTGGGGTCAAATAGTTTCATGTTCCCCAAATGGCCCAAAA |
| | | CTGACAGTTTAAACGCTGTCTTGGAACCTAATATGACAAAAGCGTGATCTCATCCAA |
| | | GATGAACTAAGTTTGGTTCGTTGAAATGCTAACGGCCAGTTGGTCAAAAAGAAACTT |
| | | CCAAAAGTCGGCATACCGTTTGTCTTGTTTGGTATTGATTGACGAATGCTCAAAAATA |
| | | ATCTCATTAATGCTTAGCGCAGTCTCTCTATCGCTTCTGAACCCCGGTGCACCTGTGC |
| | | CGAAACGCAAATGGGGAAACACCCGCTTTTTGGATGATTATGCATTGTCTCCACATT |
| | | GTATGCTTCCAAGATTCTGGTGGGAATACTGCTGATAGCCTAACGTTCATGATCAAA |
| | | ATTTAACTGTTCTAACCCCTACTTGACAGCAATATATAAACAGAAGGAAGCTGCCCT |
| | | GTCTTAAACCTTTTTTTTTATCATCATTATTAGCTTACTTTCATAATTGCGACTGGTTC |
| | | CAATTGACAAGCTTTTGATTTTAACGACTTTTAACGACAACTTGAGAAGATCAAAAA |
| | | ACAACTAATTATTGGATCCCGA |
| DAS1 | SEQ ID NO: 32 | AAATCTGAACACGATGAAACCTCCCCGTAGATTCCACCGCCCCGTTACTTTTTTGGGC |
| | | AATCCCGTTGATAAGATCCATTTTAGAGTTGTTTCTGAAAGGATTACAGGCGTTGAA |
| | | GGGTCAGAGAGATGCCAGAGAACAGACCAATTGGTAGTTTGCTAAAGTGGACGTCT |
| | | GGCAGGTGCTCTATCGTGTTCTTTATTTAGGGCGTTACACTTAGTAGGATTACGTAAC |
| | | AATTTGGCTTAACCTTCTAAGTTAGAAAGAAACCAAGAGGGGTCCTCTTTAACGTTC |
| | | AGCAGTATCTAAAACACAAAACCTGCCCTCATAATACATCATTCTATCTGTCAAGCT |
| | | GTGCTACCCCACAGAAATACCCCCAAGAGTTAAAGTGAAAAGAAAAGCTAAATCTG |
| | | TTAGACTTCACCCCATAACAAACTTGATAGTTCCTGTAGCCAATGAAAGTTAACCCC |
| | | ATTCAATGTTCCGAGATCTAGTATGCTTGCTCCTATAAGGAACGAAGGGTTCCAGCTT |
| | | CCTTACCCCATCAATGGAAATCTCCTATTTACCCCCCACTGGAAAGATCCGTCCGAAC |
| | | GAACGGATAATAGAAAAAAGAAATTCGGACAAAATAGAACACTTATTTAGCCAATG |
| | | AAATCCATTTCCAGCATCTCCTTCAACTGCCGTTCCATCCCCTTTGTTGAGCTACACC |
| | | ATCGTCAGCCAGTACCGAATAGGAAACTTAACCGATATCTTGGAGAATTCTAATGCG |
| | | CGAATGAGTTTAGCCTAGATATCCTTAGTGAAGGGTTGTTCCGATACTTCTCCACATT |
| | | CAGTCATTTCAGATGGGCAGCATTGTTATCATGAAGAAACGGAAACGGGCAGTAAG |
| | | GGTTAACCGCCAAATTATATAAAGACAACATGTCCCCAGTTTAAAGTTTTTCTTTCCT |
| | | ATTCTTGTATCCTGAGTGACCGTTGTGTTTAAAATAACAAGTTCGTTTTAACTTAAGA |
| | | CCAAAACCAGTTACAACAAATTATTCCCCAACTAAACACTAAAGTTCACTCTTATCA |
| | | AACTATCAAACATCAAAG |
| DAS2 | SEQ ID NO: 33 | CCTGTTGATAAGACGCATTCTAGAGTTGTTTCATGAAAGGGTTACGGGTGTTGATTG |
| | | GTTTGAGATATGCCAGAGGACAGATCAATCTGTGGTTTGCTAAACTGGAAGTCTGGT |
| | | AAGGACTCTAGCAAGTCCGTTACTCAAAAAGTCATACCAAGTAAGATTACGTAACAC |
| | | CTGGGCATGACTTTCTAAGTTAGCAAGTCACCAAGAGGGTCCTATTTAACGTTTGGC |
| | | GGTATCTGAAACACAAGACTTGCCTATCCCATAGTACATCATATTACCTGTCAAGCT |
| | | ATGCTACCCCACAGAAATACCCCAAAAGTTGAAGTGAAAAAATGAAAATTACTGGT |
| | | AACTTCACCCCATAACAAACTTAATAATTTCTGTAGCCAATGAAAGTAAACCCCATT |
| | | CAATGTTCCGAGATTTAGTATACTTGCCCCTATAAGAAACGAAGGATTTCAGCTTCCT |
| | | TACCCCATGAACAGAAATCTTCCATTTACCCCCCACTGGAGAGATCCGCCCAAACGA |
| | | ACAGATAATAGAAAAAAGAAATTCGGACAAATAGAACACTTTCTCAGCCAATTAAA |
| | | GTCATTCCATGCACTCCCTTTAGCTGCCGTTCCATCCCTTTGTTGAGCAACACCATCG |
| | | TTAGCCAGTACGAAAGAGGAAACTTAACCGATACCTTGGAGAAATCTAAGGCGCGA |
| | | ATGAGTTTAGCCTAGATATCCTTAGTGAAGGGTTGTTCCGATACTTCTCCACATTCAG |
| | | TCATAGATGGGCAGCTTTGTTATCATGAAGAGACGGAAACGGGCATTAAGGGTTAAC |
| | | CGCCAAATTATATAAAGACAACATGTCCCCAGTTTAAAGTTTTTCTTTCCTATTCTTG |
| | | TATCCTGAGTGACCGTTGTGTTTAATATAACAAGTTCGTTTTAACTTAAGACCAAAAC |
| | | CAGTTACAACAAATTATAACCCCTCTAAACACTAAAGTTCACTCTTATCAAACTATCA |
| | | AACATCAAAAGAATTCGCG |
| FLD1 | SEQ ID NO: 34 | AAATCAGCCATTAATCTCACCTCAGTTTTMAATCAGTAGAATTITCAATGAAACAA |
| | | ACGGTTGGTATATTATTTGATAGGGTAGCCAAATTTCCAAAAATGAACTTTTCATCAG |
| | | GTAATATCTTGAATACCGTAATGTAGTGACTATTGGAAGAAACTGCTATCAAATTAT |
| | | ATTTCGGATAGAAATCCAAACCCCAGACTGATCTCTTGAGTCTCAACTCTAAGTCAG |
| | | CCGCGACTCTAATTATCTGTGGATTAGGAGTTAGTGTGGACAAAGCATCAGTATAGT |
| | | ATAACTTTACGGTTCCATTATCAGACGCTATTGCAAGAACTTCCTTTCCATTGATCTC |
| | | TCCAATTCGACAGTAATTGATATCATAAGGTAGGTCTGGAAACACACTGGCGCTTGT |
| | | ATCCCATTCTGCAGGAATTTCTGGAACGGTGGTAATGGTAGTTATCCAACGGAGTTG |
| | | GGGTAGTTGGTATATCTGGATATGCCGCCTATAGGATAAAAACAGGAGAGAGTGAA |
| | | CCTTGCTTACGGCTACTAGATTGTTCTTGTACTCGGAATTGTCGTTATCGGAAACTAG |
| | | ACTAATCTCATCTGTGTGTTGCAGTACTATTGAGTCGTTGTAGTATCTACCAGGAGGG |
| | | CATTCCATGAACTAGTGAGACAAATGAGTTGGATTTTCTCAATAGACATATGCAAGA |
| | | ATGCTACACAACGGATGTCGCACTCTTTTTCTTAGTTGATAATATCATCCAATCAGAA |
| | | GACACGGGCTAGAAGGACTTGCTCCCGAAGGATAATCCACTGCTACTATCTCCCTTV |
| | | CTCACATATAGTCTTGCAGGGCTCATGCCCCTTTCTCCTTCGAACTGCCCGATGAGGA |
| | | AGTCTTTAGCCTATCAAGGAATTCGGGACCATCATCAATTTTTAGAGCCTTACCTGAT |
| | | CGCAATCAGGATTIVACTACTCATATAAATACATCACTCAAACTCCAACTTTGCTTGT |
| | | TCATACAATTCTTGATATTCACAGGATC |
| PEX8 | SEQ ID NO: 35 | AAATTAACCAGTGTTTTCTTATCTATTTGTCTTTTTACACTAAAGTGAAGTACGAATC |
| | | CATGCGATTGATTCCTCCTCAGATATCAGCTGAATTCTTGCTTATGTAATACTTGCGC |
| | | GAACTACATGTGAACTTAGGATTCGATAAGGCTGGGGGGTCAACCAACCCCACTTCA |
| | | AAGAGCCGACCCGTATAAATAGCCTCTGCGTCCTCAGATCAACAAGACGAAGCAATT |
| | | TTTTTTTACCTATCTTCAGGTGCCTGTTAG |
| SHB17 | SEQ ID NO: 36 | AAATTCTTTTTACGTGGTGCGCATACTGGACAGAGGCAGAGTCTCAATTTCTTCTTTT |
| | | GAGACAGGCTACTACAGCCTGTGATTCCTCTTGGTACTMGATTTGCTTTTATCTGGC |
| | | TCCGTTGGGAACTGTGCCTGGGTTTTGAAGTATCTTGTGGATGTGTTTCTAACACTTT |
| | | TTCAATCTTCTTGGAGTGAGAATGCAGGACTTTGAACATCGTCTAGCTCGTTGGTAGG |
| | | TGAACCGTTTTACCTTGCATGTGGTTAGGAGTTTTCTGGAGTAACCAAGACCGTCTTA |
| | | TCATCGCCGTAAAATCGCTCTTACTGTCGCTAATAATCCCGCTGGAAGAGAAGTTCG |
| | | AACAGAAGTAGCACGCAAAGCTCTTGTCAAATGAGAATTGTTAATCGTTTGACAGGT |
| | | CACACTCGTGGGCTATGTACGATCAACTTGCCGGCTGTTGCTGGAGAGATGACACCA |
| | | GTTGTGGCATGGCCAATTGGTATTCAGCCGTACCACTGTATGGAAAATGAGATTATC |
| | | TTGTTCTTGATCTAGTTTCTTGCCATTTTAGAGTTGCCACATTCGTAGGTTTCAGTACC |
| | | AATAATGGTAACTTCCAAACTTCCAACGCAGATACCAGAGATCTGCCGATCCTTCCC |
| | | CAACAATAGGAGCTTACTACGCCATACATATAGCCTATCTATTTTCACTTTCGCGTGG |
| | | GTGCTTCTATATAAACGGTTCCCCATCTTCCGTITCATACTACTTGAATTTTAAGCACT |
| | | AAAGAATT |
| FGH1 | SEQ ID NO: 37 | GTGAATTTGTCACGGAATTGACCAAGAGGTCAGACGATCCTGTATCCCATTGAGCCG |
| | | TTATGCTTTGTGGGGGAAACCCTATTTCTATCGTACTAAGAAAACCAATGGTGAACT |
| | | CATATTCGGTATCAATGGCGACGATTCCAGCATAGCCTGTAGACAGTAACAACACTA |
| | | GGGCAACAGCAACTAACATATCTTCATTGATGAAACGTTGTGATCGGTGTGACTTTT |
| | | ATAGTAAAAGCTACAACTGTTTGAAATACCAAGATATCATTGTGAATGGCTCAAAAG |
| | | GGTAATACATCTGAAAAACCTGAAGTGTGGAAAATTCCGATGGAGCCAACTCATGAT |
| | | AACGCAGAAGTCCCATTTTGCCATCTTCTCTTGGTATGAAACGGTAGAAAATGATCC |
| | | GAGTATGCCAATTGATACTCTTGATTCATGCCCTATAGTTTGCGTAGGGTTTAATTGA |
| | | TCTCCTGGTCTATCGATCTGGGACGCAATGTAGACCCCATTAGTGGAAACACTGAAA |
| | | GGGATCCAACACTCTAGGCGGACCCGCTCACAGTCATTTCAGGACAATCACCACAGG |
| | | AATCAACTACTTCTCCCAGTCTTCCTTGCGTGAAGCTTCAAGCCTACAACATAACACT |
| | | TCTTACTTAATCTTTGATTCTCGAATTGTTTACCCAATCTTGACAACTTAGCCTAAGC |
| | | AATACTCTGGGGTTATATATAGCAATTGCTCTTCCTCGCTGTAGCGTTCATTCCATCT |
| | | TTCTAGAATTCGT |
| Methanol | SEQ ID NO: 38 | CTTCCCCATTTCACTGACAGTTTGTAGAAATAGGGCAACAATTGATGCAAATCGATTT |
| inducible | | TCAACGCATTGGTTTTGATAGCATTGATGATCTTGGAGCTGTAAAAGTCCGGCTGGA |
| promoter | | TAAGCTCAATGAAATAGGTTGGTTGATCTGGATCTTCTTTTGGGTCATTTTGTTCGCT |
| | | CTGTATTTCACAAATTGCCAGAATCTCTGCCAACCACAGTGGTAGGTCCAACTTGGT |
| | | GTTCTGAATCACAGGCTTCCCCGGGTTGTTCTCTAAATAACCGAGGCCCGGCACAGA |
| | | AATCGTAAACCGACACGGTATCTTTTGTCCGTCCGCCAGTATCTCATCAAGGTCGTAG |
| | | TAGCCCATGATGAGTATCAAAGGGGATTTGGTTATGCGATGCAACGAGAGATTGTTT |
| | | ATCCCAGATGCTGATGTAAAAACCTTAACCAGCGTGACAGTAGAAATAAGACACGTT |
| | | AAAATTACCCGCGCTTCCCTAACAATTGGCTCTGCCTTTCGGCAAGTTTCTAACTGCC |
| | | CTCCCCTCTCACATGCACCACGAACTTACCGTTCGCTCCTAGCAGAACCACCCCAAA |
| | | GTTTAATCAGGACCGCATTTTAGCCTATTGCTGTAGAACCCCACAACATAACCTGGTC |
| | | CAGAGCCAGCCCTTTATATATGGTAAATCCCGTTTGAACTTCGAAGTGGAATCGGAA |
| | | TTTTTACATCAAAGAAACTGATACTGAAACTTTTGGCTTCGACTTGGACTTTCTCTTA |
| | | ATCGAATTCGT |
| PMP20 | SEQ ID NO: 39 | ACACAGTTATTATTCATTTAAATGTCAAAACAGTAGTGATAAAAGGCTATGAAGGAG |
| | | GTTGTCTAGGGGCTCGCGGAGGAAAGTGATTCAAACAGACCTGCCAAAAAGAGAAA |
| | | AAAGAGGGAATCCCTGTTCTTTCCAATGGAAATGACGTAACTTTAACTTGAAAAATA |
| | | CCCCAACCAGAAGGGTTCAAACTCAACAAGGATTGCGTAATTCCTACAAGTAGCTTA |
| | | GAGCTGGGGGAGAGACAACTGAAGGCAGCTTAACGATAACGCGGGGGGATTGGTGC |
| | | ACGACTCGAAAGGAGGTATCTTAGTCTTGTAACCTCTTTTTTCCAGAGGCTATTCAAG |
| | | ATTCATAGGCGATATCGATGTGGAGAAGGGTGAACAATATAAAAGGCTGGAGAGAT |
| | | GTCAATGAAGCAGCTGGATAGATTTCAAATTTTCTAGATTTCAGAGTAATCGCACAA |
| | | AACGAAGGAATCCCACCAAGACAAAAAAAAAAATTCTAAGG AATTCCGAAACG |
| DAK2 | SEQ ID NO: 40 | AAATAAGCATGTTTGTTTCAGATCAAAGATTAGCGTTTCAAAGTTGTGGAAAAGTGA |
| | | CCATGCAACAATATGCAACACATTCGGATTATCTGATAAGTTTCAAAGCTACTAAGT |
| | | AAGCCCGTTTCAAGTCTCCAGACCGACATCTGCCATCCAGTGATTTTCTTAGTCCTGA |
| | | AAAATACGATGTGTAAACATAAACCACAAAGATCGGCCTCCGAGGTTGAACCCTTAC |
| | | GAAAGAGACATCTGGTAGCGCCAATGCCAAAAAAAAATCACACCAGAAGGACAATT |
| | | CCCTTCCCCCCCAGCCCATTAAAGCTTACCATTTCCTATTCCAATACGTTCCATAGAG |
| | | GGCATCGCTCGGCTCATTTTCGCGTGGGTCATACTAGAGCGGCTAGCTAGTCGGCTG |
| | | TTTGAGCTCTCTAATCGAGGGGTAAGGATGTCTAATATGTCATAATGGCTCACTATAT |
| | | AAAGAACCCGCTTGCTCAACCTTCGACTCCTTTCCCGATCCTTTGCTTGTTGCTTCTTC |
| | | TTTTATAACAGGAAACAAAGGAATTTATACACTTTAAGAATT |
| GCW14 | SEQ ID NO: 41 | CAGGTGAACCCACCTAACTATTTTTAACTGGCATCCAGTGAGCTCGCTGGGTGAAAG |
| | | CCAACCATCTTTTGTTTCGGGGAACCGTGCTCGCCCCGTAAAGTTAATTTTTTTTTCCC |
| | | GCGCAGCTTTAATCTTTCGGCAGAGAAGGCGTTTTCATCGTAGCGTGGGAACAGAAT |
| | | AATCAGTTCATGTGCTATACAGGCACATGGCAGCAGTCACTATTTTGCTTTTTAACCT |
| | | TAAAGTCGTTCATCAATCATTAACTGACCAATCAGATTTTTTGCATTTGCCACTTATC |
| | | TAAAAATACTTTTGTATCTCGCAGATACGTTCAGTGGTTTCCAGGACAACACCCAAA |
| | | AAAAGGTATCAATGCCACTAGGCAGTCGGTTTTATTTTTGGTCACCCACGCAAAGAA |
| | | GCACCCACCTCTTTTAGGTTTTAAGTTGTGGGAACAGTAACACCGCCTAGAGCTTCA |
| | | GGAAAAACCAGTACCTGTGACCGCAATTCACCATGATGCAGAATGTTAATTTAAACG |
| | | AGTGCCAAATCAAGATTTCAACAGACAAATCAATCGATCCATAGTTACCCATTCCAG |
| | | CCTTTTCGTCGTCGAGCCTGCTTCATTCCTGCCTCAGGTGCATAACTTTGCATGAAAA |
| | | GTCCAGATTAGGGCAGATTTTGAGTTTAAAATAGGAAATATAAACAAATATACCGCG |
| | | AAAAAGGTTTGTTTATAGCTTTTCGCCTGGTGCCGTACGGTATAAATACATACTCTCC |
| | | TCCCCCCCCTGGTTCTCTTTTTCTTTTGTTACTTACATTTTACCGTTCCGT |
| FDH1 | SEQ ID NO: 42 | AAATAAATGGCAGAAGGATCAGCCTGGACGAAGCAACCAGTTCCAACTGCTAAGTA |
| | | AAGAAGATGCTAGACGAAGGAGACTTCAGAGGTGAAAAGTTTGCAAGAAGAGAGCT |
| | | GCGGGAAATAAATTTTCAATTTAAGGACTTGAGTGCGTCCATATTCGTGTACGTGTCC |
| | | AACTGTTTTCCATTACCTAAGAAAAACATAAAGATTAAAAAGATAAACCCAATCGGG |
| | | AAACTTTAGCGTGCCGTTTCGGATTCCGAAAAACTTTTGGAGCGCCAGATGACTATG |
| | | GAAAGAGGAGTGTACCAAAATGGCAAGTCGGGGGCTACTCACCGGATAGCCAATAC |
| | | ATTCTCTAGGAACCAGGGATGAATCCAGGTTTTTGTTGTCAGGTAGGTCAAGCATT |
| | | CACTTCTTAGGAATATCTCGTTGAAAGCTACTTGAAATCCCATTGGGTGCGGAACCA |
| | | GCTTCTAATTAAATAGTTCGATGATGTTCTCTAAGTGGGACTCTACGGCTCAAACTTC |
| | | TACACAGCATCATCTTAGTAGTCCCTTCCCAAAACACCATTCTAGGTTTCGGAACGTA |
| | | ACGAAACAATGTTCCTCTCTTCACATTGGGCCGTTACTCTAGCCTTCCGAAGAACCAA |
| | | TAAAAGGGACCGGCTGAAACGGGTGTGGAAACTCCTGTCCAGTTTATGGCAAAGGCT |
| | | ACAGAAATCCCAATCTTGTCGGGATGTTGCTCCTCCCAAACGCCATATTGTACTGCA |
| | | GTTGGTGCGCATTTTAGGGAAAATTTACCCCAGATGTCCTGATTTTCGAGGGCTACCC |
| | | CCAACTCCCTGTGCTTATACTTAGTCTAATTCTATTCAGTGTGCTGACCTACACGTAA |
| | | TGATGTCGTAACCCAGTTAAATGGCCGAAAAACTATTTAAGTAAGTTTATTTCTCCTC |
| | | CAGATGAGACTCTCCTTCTTTTCTCCGCTAGTTATCAAACTATAAACCTATTTTACCTC |
| | | AAATACCTCCAACATCACCCACTTAAACAGAATT |
| FBA1 | SEQ ID NO: 43 | TGCTTAAGTAATTGAAAACAGTGTTGTGATTATATAAGCATGGTATTTGAATAGAAC |
| | | TACTGGGGTTAACTTATCTAGTAGGATGGAAGTTGAGGGAGATCAAGATGCTTAAAG |
| | | AAAAGGATTGGCCAATATGAAAGCCATAATTAGCAATACTTATTTAATCAGATAATT |
| | | GTGGGGCATTGTGACTTGACTTTTACCAGGACTTCAAACCTCAACCATTTAAACAGTT |
| | | ATAGAAGACGTACCGTCACTTTTGCTTTTAATGTGATCTAAATGTGATCACATGAACT |
| | | CAAACTAAAATGATATCTTTTACTGGACAAAAATGTTATCCTGCAAACAGAAAGCTT |
| | | TCTTCTATTCTAAGAAGAACATTTACATTGGTGGGAAACCTGAAAACAGAAAATAAA |
| | | TACTCCCCAGTGACCCTATGAGCAGGATTTTTGCATCCCTATTGTAGGCCTTTCAAAC |
| | | TCACACCTAATATTTCCCGCCACTCACACTATCAATGATCACTTCCCAGTTCTCTTCTT |
| | | CCCCTATTCGTACCATGCAACCCTTACACGCCTTTTCCATTTCGGTTCGGATGCGACT |
| | | TCCAGTCTGTGGGGTACGTAGCCTATTCTCTTAGCCGGTATTTAAACATACAAATTCA |
| | | CCCAAATTCTACCTTGATAAGGTAATTGATTAATTTCATAAATGAATTCGCG |
| GAP | SEQ ID No: 44 | TTTTTGTAGAAATGTCTTGGTGTCCTCGTCCAATCAGGTAGCCATCTCTGAAATATCT |
| | | GGCTCCGTTGCAACTCCGAACGACCTGCTGGCAACGTAAAATTCTCCGGGGTAAAAC |
| | | TTAAATGTGGAGTAATGGAACCAGAAACGTCTCTTCCCTTCTCTCTCCTTCCACCGCC |
| | | CGTTACCGTCCCTAGGAAATTTTACTCTGCTGGAGAGCTTCTIVTACGGCCCCCTTGC |
| | | AGCAATGCTCTTCCCAGCATTACGTTGCGGGTAAAACGGAGGTCGTGTACCCGACCT |
| | | AGCAGCCCAGGGATGGAAAAGTCCCGGCCGTCGCTGGCAATAATAGCGGGCGGACG |
| | | CATGTCATGAGATTATTGGAAACCACCAGAATCGAATATAAAAGGCGAACACCTTTC |
| | | CCAATTTMGTTTCTCCTGACCCAAAGACTTTAAATTTAATTFATTTGTCCCTATTTCA |
| | | ATCAATTGAACAACTAT |
| PGK | SEQ ID No: 45 | AAATAGCAGTTTGCGGTTTCTTGATTTCATGGGGGGAACAAACAATAGTGTTGCCTT |
| | | AATTCTAATTGGCATTGTTGCTTGGAATCGAAATTGGGGGATAACGTCATATCTGAA |
| | | AAGTAAACAACTTCGGGAAATCAGGCTGTTTGAATGGCTTGGAAGCGAGATAGAAA |
| | | GGGGATAGCGAGATAGAGGGGGCGGAGTAGACGAAGGGTGTTAAACTGCTGAAATC |
| | | TCTCAATCTGGAAGAAACGGAATAAATTAACTCCTTGCGATAATAAAATCCGAGTCC |
| | | GTTATGACCCCACACCGTGTTGACCACGGCATACCCCATGGAATCTGGTACAAAGCG |
| | | TCAGTCTTGAAGACACCATCACGTGTAGGAGACTGATTGTCTGACCGTCCAGCAAAA |
| | | AGGGCATTATAAATCTTGCTGTTAAAGGGGTGAGGGGAGATGCAGGTTGTTCTTTTA |
| | | TTCGCCTTGAACTTTITAATTTTCCCGGGGTTGCGGAGCGTGAACAGTTAGCCCGATC |
| | | TGATAGCTTGCAAGATTCAACAGTTTATCCACTACAGGTCAGAGAGATCGCCGCAGA |
| | | AGAAATGCTCGTCTCGTGTTCCAGCACACATACTGGTGAAGTCGTTATTTTGCCGAA |
| | | GGGGGGGTAATAAGGTTATGCACCCCCTCTCCACACCCCAGAATCATTTTTTAGCTG |
| | | GGTTCAAGGCATTAGACTTTGCACATTTTTCCCTTAAACACCCTTGAAACGCGGATAA |
| | | ACAGTTGCATGTGCATCCTAAAACTAGGTGAGATGCGTACTCCGTGCTCCGATAATA |
| | | ACAGTGGTGTTGGGGTTGCTGCTAGCTCACGCACTCCGTTCTTTTTTTTCAACCAGCA |
| | | AAATTCGATGGGGAGAAACTTGGGGTACTTTGCCGACTCCTCCACCATGCTGGTATA |
| | | TAAATAATACTCGCCCACTTTTCGTTTGCTGCTTTTATATTTCATAGACTGAAAAAGA |
| | | CTCTTCTTCTACTTTTTCATAATATATCTCAGATATCACTACTATAG |
| AOX2_PRO | SEQ ID NO: 46 | cgcATTTAAATtgacttccttacaaaggggcttctgtttttgaggttccagttttctc |
| | | ataaactccaaccctgtagctctctctaatgcttctaatggtacttcaaaatctgtga |
| | | gtttgacagaatttggtattggctcgtttggaaggacgaaagctgccagcgcaacatc |
| | | accagggtttcgtctattcttcgggtcctcggctacgaccaatttaaagaaatgcgtc |
| | | ggcactgcaactgatggcggacttccaatgagttcatatgttaccttccatttaccat |
| | | cattaccatcctgcttaggcaaaaaaagaggacctgtaacaatgcgaactgatcgaaa |
| | | atattgagttagagtacgagtaaagtactccaaatgagcccaataatctctgttaaaa |
| | | ccatctccaacttggggtgacatgttggtcaaaaaaaagtttcatccattgcgttttg |
| | | agagaacttagcgtttgccgctggtgcttgatgccctctatcataaccagatcgaaaa |
| | | tagtcctttaatcttgccctaaatatgcttggaatttgctcatcttccttaaaaaaac |
| | | aattctttctatcagcattgtgactggctaaagaatctggggtcaaatgttcaacgac |
| | | ataatatggattccgggtttgacggttgtatactgagacaaattctgctctggtttgt |
| | | aaatcatggatgggaccaggaaaaccatacttgaaaaaatcagaaggtctcactattg |
| | | gagtctctagcgaaacagatgttgttggaggagataatgagctaggacttatggtagt |
| | | tggatttgcaactatagtgtcctttgccttactccaaaacattgatctggcaaaagct |
| | | gagtatatagggaaagttactggtggaattgactaacctgcttagtttctggagcgcg |
| | | ctaaaacttcaattctttttccccgcgacaaaactttcaagtgtttgaaaccaaagct |
| | | agcaccttcgaatagtcaaattagcGAATTCgcg |
| TEFg_PRO | SEQ ID NO: 47 | GCGatttaaattcgcgaaagaacagcctaataaactccgaagcatgatggcctctatc |
| | | cggaaaacgttaagagatgtggcaacaggagggcacatagaatttttaaagacgctga |
| | | agaatgctatcatagtccgtaaaaatgtgatagtactttgtttagtgcgtacgccact |
| | | tattcggggccaatagctaaacccaggtttgctggcagcaaattcaactgtagattga |
| | | atctctctaacaataatggtgttcaatcccctggctggtcacggggaggactatcttg |
| | | cgtgatccgcttggaaaatgttgtgtatccctttctcaattgcggaaagcatctgcta |
| | | cttcccataggcaccagttacccaattgatatttccaaaaaagattaccatatgttca |
| | | tctagaagtataaatacaagtggacattcaatgaatatttcattcaattagtcattga |
| | | cactttcatcaacttactacgtcttattcaacaatGAATTCgcg |
| | SEQ ID NO: 48 | MQVKSIVNLLLACSLAVA |
| | SEQ ID NO: 49 | MQFNWMKTVASILSALTLAQA |
| | SEQ ID NO: 50 | MYRNLIIATALTCGAYSAYVPSEPWSTLTPDASLESALKDYSQTFGIAIKSLDADKIKR |
| | SEQ ID NO: 51 | MNLYLITLLFASLCSAITLPKR |
| | SEQ ID NO: 52 | MFEKSKFVVSFLLLLQLFCVLGVHG |
| | SEQ ID NO: 53 | MQFNSVVISQLLLTLASVSMG |
| | SEQ ID NO: 54 | MKSQLIFMALASLVASAPLEHQQQHHKHEKR |
| | SEQ ID NO: 55 | MKFAISTLLIILQAAAVFA |
| | SEQ ID NO: 56 | MKLLNFLLSFVTLFGLLSGSVFA |
| | SEQ ID NO: 57 | MIFNLKTLAAVAISISQVSA |
| | SEQ ID NO: 58 | MKISALTACAVTLAGLAIAAPAPKPEDCTTTVQKRHQHKR |
| | SEQ ID NO: 59 | MSYLKISALLSVLSVALA |
| | SEQ ID NO: 60 | MLSTILNIFILLLFIQASLQ |
| | SEQ ID NO: 61 | MKLSTNLILAIAAASAVVSAAPVAPAEEAANHLHKR |
| | SEQ ID NO: 62 | MFKSLCMLIGSCLLSSVLA |
| | SEQ ID NO: 63 | MKLAALSTIALTILPVALA |
| | SEQ ID NO: 64 | MSFSSNVPQLFLLLVLLTNIVSG |
| | SEQ ID NO: 65 | MQLQYLAVLCALLLNVQSKNVVDFSRFGDAKISPDDTDLESRERKR |
| | SEQ ID NO: 66 | MKIHSLLLWNLFFIPSILG |
| | SEQ ID NO: 67 | MSTLTLLAVLLSLQNSALA |
| | SEQ ID NO: 68 | MINLNSFLILTVTLLSPALALPKNVLEEQQAKDDLAKR |
| | SEQ ID NO: 69 | MFSLAVGALLLTQAFG |
| | SEQ ID NO: 70 | MKILSALLLLFTLAFA |
| | SEQ ID NO: 71 | MKVSTTKFLAVFLLVRLVCA |
| | SEQ ID NO: 72 | MQFGKVLFAISALAVTALG |
| | SEQ ID NO: 73 | MWSLFISGLLIFYPLVLG |
| | SEQ ID NO: 74 | MRNHLNDLVVLFLLLTVAAQA |
| | SEQ ID NO: 75 | MFLKSLLSFASILTLCKA |
| | SEQ ID NO: 76 | MFVFEPVLLAVLVASTCVTA |
| | SEQ ID NO: 77 | MVSLRSIFTSSILAAGLTRAHG |
| | SEQ ID NO: 78 | MFSPILSLEIILALATLQSVFA |
| | SEQ ID NO: 79 | MIINHLVLTALSIALA |
| | SEQ ID NO: 80 | MLALVRISTLLLLALTASA |
| | SEQ ID NO: 81 | MRPVLSLLLLLASSVLA |
| | SEQ ID NO: 82 | MVLIQNFLPLFAYTLFFNQRAALA |
| | SEQ ID NO: 83 | MKFPVPLLFLLQLFFIIATQG |
| | SEQ ID NO: 84 | MVSLTRLLITGIATALQVNA |
| | SEQ ID NO: 85 | MIFDGTTMSIAIGLLSTLGIGAEA |
| | SEQ ID NO: 86 | MVLVGLLTRLVPLVLLAGTVLLLVFVVLSGG |
| | SEQ ID NO: 87 | MLSILSALTLLGLSCA |
| | SEQ ID NO: 88 | MRLLHISLLSIISVLTKANA |
| | SEQ ID NO: 89 | MRFPSIFTAVLFAASSALAAPVNTTTEDETAQIPAEAVIGYLDLEGDFDVAVLPFSNS |
| | | TNNGLLFINTTIASIAAKEEGVSLDKREAEA |
| | SEQ ID NO: 90 | MFKSVVYSILAASLANA |
| | SEQ ID NO: 91 | MLLQAFLFLLAGFAAKISA |
| | SEQ ID NO: 92 | MASSNLLSLALFLVLLTHANS |
| | SEQ ID NO: 93 | MNIFYIFLFLLSFVQGLEHTHRRGSLVKR |
| | SEQ ID NO: 94 | MLIIVLLFLATLANSLDCSGDVFFGYTRGDKTDVHKSQALTAVKNIKR |
| | SEQ ID NO: 95 | MESVSSLFNIFSTIMVNYKSLVLALLSVSNLKYARGMPTSERQQGLEER |
| | SEQ ID NO: 96 | MFAFYFLTACISLKGVFG |
| | SEQ ID NO: 97 | MRFSTTLATAATALFFTASQVSA |
| | SEQ ID NO: 98 | MKFAYSLLLPLAGVSASVINYKR |
| | SEQ ID NO: 99 | MKFFAIAALFAAAAVAQPLEDR |
| | SEQ ID NO: 100 | MQFFAVALFATSALA |
| | SEQ ID NO: 101 | MKWVTFISLLFLFSSAYSRGVFRR |
| | SEQ ID NO: 102 | MRSLLILVLCFLPLAALG |
| | SEQ ID NO: 103 | MKVLILACLVALALA |
| | SEQ ID NO: 104 | MFNLKTILISTLASIAVA |
| | SEQ ID NO: 105 | MYRKLAVISAFLATARAQSA |
| WT | SEQ ID NO: 106 | MRFPSIFTAVLFAASSALAAPVNTTTEDETAQIPAEAVIGYLDLEGDFDVAVLPFSNS |
| | | TNNGLLFINTTIASIAAKEEGVQLDKR |
| App3 | SEQ ID NO: 107 | MRFPPIFTAALFAASSALAAPANTTTEDETAQIPAEAVIGYLDSEGDSDVAVLPFSNS |
| | | TNNGLSFINTTIASIAAKEEGVQLDKR |
| App8 | SEQ ID NO: 108 | MRFPSIFTAVLFAASSALAAPANTTTEDETAQIPAEAVISYSDLEGDFDAAALPLSNS |
| | | TNNGLSSTNTTIASIAAKEEGVQLDKR |
| App9 | SEQ ID NO: 109 | MRPPSIFTAVLFAASSALAAPANTTTEDETTQIPAEAVATYLDLEGDVDVAVLPFSSS |
| | | TNNGLSFINTTIASIAAKEEGVQLDKR |
| App10 | SEQ ID NO: 110 | MRFPSIFFAALFAASSALAAPANTTTEGETAQTPAEAVIGYRDLEGDFDVAVLPFPNS |
| | | TNNGLLFTNTTTASIAAKEEGVQLDKR |
| appS1 | SEQ ID NO: 111 | MRFPSIFTAVLLAAPSALAAPANATTEDEAAQIPAEAVIGYLDLEGDFDAAVLPFSNS |
| | | TNNGLLSINTTIASIAAKEEGVQLDKR |
| appS4 | SEQ ID NO: 112 | MRFPSIFTAVVFAASSALAAPANTTAEDETAQIPAEAVIGYLGLEGDSDVAALPLSDS |
| | | TNNGSLSTNTTIASIAAKEEGVQLDKR |
| appS6 | SEQ ID NO: 113 | MRLPSIFTAAVFAASSALAAPANTTTEDETAQIPAEAAIGYLDLEGDSDVAVLPLSNS |
| | | TNNGLLFINTTIASIAAKEEGVQLDKR |
| appS8 | SEQ ID NO: 114 | MRFPSIFTAVLFAASSALAAPANTTTEDETAQIPAEAVIGYLDLEGDFDVAVLPFSNS |
| | | TNDGLSFINTTTASIAAKEEGVQLDKR |
| a-Factor | SEQ ID NO: 115 | MRFPSIFTAVLFAASSALAAPVNTTTEDETAQIPA |
| PpScw11p | SEQ ID NO: 116 | MLSTILNIFILLLFIQASLQ APIPVVTKYVTEGIANV |
| PpDse4p | SEQ ID NO: 117 | MSFSSNVPQLFLLLVLLTNIVSGAVISVWSTSKVTK |
| PpExg1p | SEQ ID NO: 118 | MNLYLITLLFASLCSAITLPKRDIIWDYSSEKIMG |
| a-EGFP | SEQ ID NO: 119 | MRFPSIFTAVLFAASSALAAPVNTTTEDETAQIPA |
| S-EGFP | SEQ ID NO: 120 | MLSTILNIFILLLFIQASLQEFDYKDDDDKMVSKG |
| D-EGFP | SEQ ID NO: 121 | MSFSSNVPQLFLLLVLLTNIVSGEFDYKDDDDKMV |
| E-EGFP | SEQ ID NO: 122 | MNLYLITLLFASLCSAEFDYKDDDDKMVSKGEELF |
| a-CALB | SEQ ID NO: 123 | MRFPSIFTAVLFAASSALAAPVNTTTEDETAQIPA |
| S-CALB | SEQ ID NO: 124 | MLSTILNIFILLLFIQASLQEFLPSGSDPAFSQPK |
| D-CALB | SEQ ID NO: 125 | MSFSSNVPQLFLLLVLLTNIVSGEFLPSGSDPAFS |
| E-CALB | SEQ ID NO: 126 | MNLYLITLLFASLCSAEFLPSGSDPAFSQPKSVLD |
| Amylase (AA) | SEQ ID NO: 127 | MVAWWSLFLYGLQVAAPALAAEVDCSRFPNATDKEGKDVLVCNKDLRPICGTDGVTY |
| | | TNDCLLCAYSIEFGTNISKEHDGECKETVPMNCSSYANTTSEDGKVMVLCNRAFNPVCG |
| | | TDGVTYDNECLLCAHKVEQGASVDKRHDGGCRKELAAVSVDCSEYPKPDCTAEDRPLC |
| | | GSDNKTYGNKCNFCNAVVESNGTLTLSHFGKC |
| Alpha K (AK) | SEQ ID NO: 128 | MRFPSIFTAVLFAASSALAAPVNTTTEDELEGDFDVAVLPFSASIAAKEEGVSLEKRAE |
| | | VDCSRFPNATDKEGKDVLVCNKDLRPICGTDGVTYTNDCLLCAYSIEFGTNISKEHDGE |
| | | CKETVPMNCSSYANTTSEDGKVMVLCNRAFNPVCGTDGVTYDNECLLCAHKVEQGASVD |
| | | KRHDGGCRKELAAVSVDCSEYPKPDCTAEDRPLCGSDNKTYGNKCNFCNAVVESNGTLT |
| | | LSHFGKC |
| Alpha T (AT) | SEQ ID NO: 129 | MRFPSIFTAVLFAASSALAAEVDCSRFPNATDKEGKDVLVCNKDLRPICGTDGVTYTND |
| | | CLLCAYSIEFGTNISKEHDGECKETVPMNCSSYANTTSEDGKVMVLCNRAFNPVCGTDG |
| | | VTYDNECLLCAHKVEQGASVDKRHDGGCRKELAAVSVDCSEYPKPDCTAEDRPLCGSD |
| | | NKTYGNKCNFCNAVVESNGTLTLSHFGKC |
| Lysozyme (LZ) | SEQ ID NO: 130 | MLGKNDPMCLVLVLLGLTALLGICQGAEVDCSRFPNATDKEGKDVLVCNKDLRPICGT |
| | | DGVTYTNDCLLCAYSIEFGTNISKEHDGECKETVPMNCSSYANTTSEDGKVMVLCNRAF |
| | | NPVCGTDGVTYDNECLLCAHKVEQGASVDKRHDGGCRKELAAVSVDCSEYPKPDCTAE |
| | | DRPLCGSDNKTYGNKCNFCNAVVESNGTLTLSHFGKC |
| Killer Protein | SEQ ID NO: 131 | MTKPTQVLVRSVSILFFITLLHLVVAAEVDCSRFPNATDKEGKDVLVCNKDLRPICGTDG |
| (KP) | | VTYTNDCLLCAYSIEFGTNISKEHDGECKETVPMNCSSYANTTSEDGKVMVLCNRAFNP |
| | | VCGTDGVTYDNECLLCAHKVEQGASVDKRHDGGCRKELAAVSVDCSEYPKPDCTAED |
| | | RPLCGSDNKTYGNKCNFCNAVVESNGTLTLSHFGKC |
| Invertase (IV) | SEQ ID NO: 132 | MLLQAFLFLLAGFAAKISAAEVDCSRFPNATDKEGKDVLVCNKDLRPICGTDGVTYTND |
| | | CLLCAYSIEFGTNISKEHDGECKETVPMNCSSYANTTSEDGKVMVLCNRAFNPVCGTDG |
| | | VTYDNECLLCAHKVEQGASVDKRHDGGCRKELAAVSVDCSEYPKPDCTAEDRPLCGSD |
| | | NKTYGNKCNFCNAVVESNGTLTLSHFGKC |
| Serum Albumin | SEQ ID NO: 133 | MKWVTFISLLFLFSSAYSAEVDCSRFPNATDKEGKDVLVCNKDLRPICGTDGVTYTNDC |
| (SA) | | LLCAYSIEFGTNISKEHDGECKETVPMNCSSYANTTSEDGKVMVLCNRAFNPVCGTDGV |
| | | TYDNECLLCAHKVEQGASVDKRHDGGCRKELAAVSVDCSEYPKPDCTAEDRPLCGSDN |
| | | KTYGNKCNFCNAVVESNGTLTLSHFGKC |
| Glucoamyl (GA) | SEQ ID NO: 134 | MSFRSLLALSGLVCSGLAAEVDCSRFPNATDKEGKDVLVCNKDLRPICGTDGVTYTNDC |
| | | LLCAYSIEFGTNISKEHDGECKETVPMNCSSYANTTSEDGKVMVLCNRAFNPVCGTDGV |
| | | TYDNECLLCAHKVEQGASVDKRHDGGCRKELAAVSVDCSEYPKPDCTAEDRPLCGSDN |
| | | KTYGNKCNFCNAVVESNGTLTLSHFGKC |
| Inulase (IN) - IC | SEQ ID NO: 135 | MKLAYSLLLPLAGVSAAEVDCSRFPNATDKEGKDVLVCNKDLRPICGTDGVTYTNDCLL |
| | | CAYSIEFGTNISKEHDGECKETVPMNCSSYANTTSEDGKVMVLCNRAFNPVCGTDGVTY |
| | | DNECLLCAHKVEQGASVDKRHDGGCRKELAAVSVDCSEYPKPDCTAEDRPLCGSDNKT |
| | | YGNKCNFCNAVVESNGTLTLSHFGKC |
| Alpha KS (AKS) | SEQ ID NO: 136 | MRFPSIFTAVLFAASSALAAPVNTTTEDELEGDFDVAVLPFSASIAAKEEGVSLEKREA |
| | | EAAEVDCSRFPNATDKEGKDVLVCNKDLRPICGTDGVTYTNDCLLCAYSIEFGTNISKE |
| | | HDGECKETVPMNCSSYANTTSEDGKVMVLCNRAFNPVCGTDGVTYDNECLLCAHKVEQG |
| | | ASVDKRHDGGCRKELAAVSVDCSEYPKPDCTAEDRPLCGSDNKTYGNKCNFCNAVVESN |
| | | GTLTLSHFGKC |
| Ovomucoid signal | SEQ ID NO: 137 | MAMAGVFVLFSFVLCGFLPDAAFG |
| peptide | | |
| Lysozyme signal | SEQ ID NO: 138 | MRSLLILVLCFLPLAALG |
| peptide | | |
| Ovalbumin Signal | SEQ ID NO: 139 | MRFPSIFTAVLFAASSALAAPVNTTTEDETAQIPAEAVIGYSDLEGDFDVAVLPFSNST |
| Peptide | | NNGLLFINTTIASIAAKEEGVSLDKREAEA |
| Ovotransferrin | SEQ ID NO: 140 | MKLILCTVLSLGIAAVCFA |
| Signal Peptide | | |
| Bovine | SEQ ID NO: 141 | MKLFVPALLSLGALGLCLA |
| Lactoferrin | | |
| Signal Peptide | | |
| Porcine | SEQ ID NO: 142 | MKLFIPALLFLGTLGLCLA |
| Lactoferrin | | |
| Signal Peptide | | |
| Kid Lipase Signal | SEQ ID NO: 143 | MESKALLLLALSVWLQSLTVSHG |
| Peptide | | |
| Porcine Lipase | SEQ ID NO: 144 | MLLIWTLSLLLGAVLG |
| Signal Peptide | | |
| XP_015135086.1 | SEQ ID NO: 145 | MYAAAAAAVAASPPRRDFISVTLSPEEAVGAGGYNNSKAWRRRSCWRKWKQLSRLQR |
| PREDICTED: | | SIILFLFAFLTVC |
| endoplasmic | | GVISYTSVREPWKSLTSKSSDEHGTEPDAPGLRLANPAVLPAPQKADANAGDYPELSPQK |
| reticulum | | PKLPHGRRNP |
| mannosyl- | | SNFQIKPPWGDVRLQTRHDTRKAVEEPAQADKQEKTEKSVISWRGAVIEPDQSSEPPSSR |
| oligosaccharide | | VKEPEKPSSV |
| 1,2-alpha- | | EGESQKEPVPINERQMAVIEAFRHAWKGYKDFAWGHDELKPLSKSYSEWFGLGLTLIDA |
| mannosidase | | LDTMWILGLRE |
| isoform X2 | | EFEEARKWVANDLAFDKNVDVNLFESTIRILGGLLSTYHLSGDSLFLEKAKDIGNRLMPA |
| [Gallus gallus] | | FKTPSKIPYS |
| | | DVNIGRGTAHPPRWTSDSTVAEVTSIQLEFRELSRLTGDEKYQKAVDEVMKHVHTLSGK |
| | | NDGLVPMFINT |
| | | NSGQFTHLGVYTLGARADSYYEYLLKQWIQGGKTENELLEDYMKAIEGVKKHLLQRSQ |
| | | PKKLTFVGELAH |
| | | GHFSAKMDHLVCFLPGTLALGAHNGLTADHMKLAEALIETCYQMYAQVETGLSPEIVH |
| | | FNLHAQKGHKDV |
| | | EIKPADRHNLLRPETVESLFYMYRFTGDKKYQDWGWEILQNFNKYTRVPTGGYTSINNV |
| | | QNPSNPEPRDK |
| | | MESEPLGETLKYMFLLFSDDIDLINLDKYVFNTEAHPLPIWVPA |
| XP_015135085.1 | SEQ ID NO: 146 | MYAAAAAAVAASPPRRDFISVTLSPEEAVGAGGYNNSKAWRRRSCWRKWKQLSRLQR |
| PREDICTED: | | SIILFLFAFLTVC |
| endoplasmic | | GVISYTSVREPWKSLTSKSSDEHGTEPDAPGLRLANPAVLPAPQKADANAGDYPELSPQK |
| reticulum | | KPKLPHGRRN |
| mannosyl- | | PSNFQIKPPWGDVRLQTRHDTRKAVEEPAQADKQEKTEKSVISWRGAVIEPDQSSEPPSS |
| oligosaccharide | | RVKEPEKPSS |
| 1,2-alpha- | | VEGESQKEPVPINERQMAVIEAFRHAWKGYKDFAWGHDELKPLSKSYSEWFGLGLTLID |
| mannosidase | | ALDTMWILGLR |
| isoform X1 | | EEFEEARKWVANDLAFDKNVDVNLFESTIRILGGLLSTYHLSGDSLFLEKAKDIGNRLMP |
| [Gallus gallus] | | AFKTPSKIPY |
| | | SDVNIGRGTAHPPRWTSDSTVAEVTSIQLEFRELSRLTGDEKYQKAVDEVMKHVHTLSG |
| | | KNDGLVPMFIN |
| | | TNSGQFTHLGVYTLGARADSYYEYLLKQWIQGGKTENELLEDYMKAIEGVKKHLLQRS |
| | | QPKKLTFVGELA |
| | | HGHFSAKMDHLVCFLPGTLALGAHNGLTADHMKLAEALIETCYQMYAQVETGLSPEIV |
| | | HFNLHAQKGHKD |
| | | VEIKPADRHNLLRPETVESLFYMYRFTGDKKYQDWGWEILQNFNKYTRVPTGGYTSINN |
| | | VQNPSNPEPRD |
| | | KMESFFLGETLKYMFLLFSDDIDLINLDKYVFNTEAHPLPIWVPA |
| XP_416490.2 | SEQ ID NO: 147 | MSAPALLPLAGRRLPALNLGASSFPHHRATLRLSEKFILLLILSAFITLCFGAFFFLPDS |
| PREDICTED: | | SKHKRFDLGL |
| mannosyl- | | EDVLIPHVDTSKGGKHLGSFLIHGQGHDEHRHREEEERLRNKIRADHEKALEEAKEKLK |
| oligosaccharide | | KSRDEIQAEIQ |
| 1,2-alpha- | | TEKNKVVQELKKKDSKPLPPVPLPNLVGINSGEPADPDIREKRNKIKEMMKHAWDNYRQ |
| mannosidase IB | | YGWGHNELKPI |
| [Gallus gallus] | | ARKGHSTNIFGNSQMGATIVDALDTLYIMGLRDEFREGQEWIDKNLDFSVNSEVSVFEV |
| | | NIRFIGGLLAA |
| | | YYLSGQEVFKIKAVQLAGKLLPAFNTPTGIPWAMVNLKSGVGRNWGWASAGSS1LAEF |
| | | GTLHMEFVHLSY |
| | | LTGDPVYYNKVMHIRKLLQKMDRPNGLYPNYLNPRTGRWGQHHTSVGGLGDSFYEYL |
| | | LKAWLMSDKTDTE |
| | | ARKMYDDALEAIEKHLIRKSNGGLTFIGEWKNGHLERKMGHLTCFAGGMFALGADGSR |
| | | DDKAGHYLQLGA |
| | | EIAHTCHESYDRTTLKLGPEAFKFDGGVEAVAVRQNEKYYILRPEVIETYWYMWRFTHD |
| | | PKYRQWGWEAT |
| | | QAIDKYCRVSGGFSGVKDVYSSSPTYDDVQQSFFLAETLKYLYLLFSNDDLLPLDNWVF |
| | | NTEAHPLPVLH |
| | | LANTTLSGNPAYR |
| XP_422293.5 | SEQ ID NO: 148 | MSGAAGCRGGGGERGPRWRRPWKLLALGLLSASSVLAAAPGAGAMSKEEKRRLGNQV |
| PREDICTED: ER | | LEMFDHAYSNYMD |
| degradation- | | IIAYPADELMPLTCRGRVRGQEPSRGDVDDALGKFSLTLIDTLDTLVVLNKTKEFEEAVK |
| enhancing alpha- | | KVIKDVNLDND |
| mannosidase- | | IVVSVFETNIRVLGGLLGGHSVAIMLKDKGEYMQWYNGELLHMAKELGYKLLPAFNTT |
| like protein 3 | | SGLPYPRVNLKF |
| isoform X2 | | GVRHPEARTGTETDTCTACAGTLILEFAALSRFTGTSIFEEYARKALDFIWEKRQRSSNLV |
| [Gallus gallus] | | GVTINIFITG |
| | | DWVRKDSGVGAGIDSYYEYLLKAYVLLGDDSFLERFNTHYDAIMKYISQPPLLLDVHIH |
| | | KPMLNARTWMD |
| | | SLLAFFPGLQVLKGDIRPAIETHEMLYQVIKKHNFLPEAFTTDFRVHWAQHPLRPEFAEST |
| | | YFLYKATGD |
| | | PYYLEVGKTLIENLNKYARVPCGFAAMKDVRTGSHEDRMDSFFLAEMFKYLYLLFADK |
| | | EDMIFDIEDYIF |
| | | TTEAHLLPLWLSTTNQTISKKNTTTEYTELDDSNFDWTCPNTQILFPNDPMFAQSIREPLK |
| | | NVVDKSCPR |
| | | SISRAEESLGTGPKPPLRARDFMASNPEHLEILKKMGVSLIHLKDGRVQLVQHAVQAASS |
| | | LDAEDGLRFM |
| | | QEMIELSSQQQKEQQLPPRAVQIVSHPFFGRVVLTAGPAQFGMDLSKHKSGTRGFVATIK |
| | | PYNGCSEITN |
| | | PEAVKEKIALMQRGQCMFAEKARNIQKAGAIGGIVIDDNEGSSSDTAPLFQMAGDGKNT |
| | | DDITIPMLFLF |
| | | NKEGNIILDAIREYEAVEVLLSDKAKDRDLEMENMDQKLSENDSHKQNSEEASSASQDV |
| | | GAVSEEPEEGE |
| | | SSDVSDLDSLPPAQADTDSVSTSDQDSSIPGPGEAGAPEPACTQGDEQPQEQQTETESDSK |
| | | VNWDNKVQP |
| | | MESILADWNEDIEAFEMMEKDEL |
| O46432.1 | SEQ ID NO: 149 | MGADARPLGVRAGGGGRGAARPGTSSRALPPPLPPLSFLLLLLAAPGARAAGYETCPMV |
| Lysosomal | | HPDMLNVHLVA |
| alpha- | | HTHDDVGWLKTVDQYFYGIFINDVQHAGVQYILDSVISSLLVEPTRRFIYVEIAFFSRWW |
| mannosidase | | HQQTNATQEVV |
| | | RDLVRQGRLEFANGGWVMNDEAATHYGAIIDQMTLGLRFLEDTFGKDGRPRVAWHIDP |
| | | FGHSREQASLFA |
| | | QMGFDGLFFGRLDYQDKRVREENLGLEQVWRASASLKPPAADLFTSVLPNIYNPPEKLC |
| | | WDTLCADKPFV |
| | | EDRRSPEYNAEELVNYFLQLATAQGQHFRTNHTIMTMGSDFQYENANMWFRNLDRLIQ |
| | | LVNAQQQANGSR |
| | | VNVLYSTPACYLWELNKANLTWSVKQDDFFPYADGPHQFWSGYFSSRPALKRYERLSY |
| | | NFLQVCNQLEAL |
| | | AGPAANVGPYGSGDSAPLNQAMAVLQHHDAVSGTSKQHVADDYARQLAAGWDPCEV |
| | | LLSNALARLSGSKE |
| | | DFTYCRNLNVSVCPLSQTAKNFQVTIYNPLGRKIDWMVRLPVSKHGFVVRDPNGTVVPS |
| | | DVVILPSSDGQ |
| | | ELLFPASVPALGFSIYSVSQVPGQRPHAHKPQPRSQRPWSRVLAIQNEHIRARFDPDTGLL |
| | | VEMENLDQN |
| | | LLLPVRQAFYWYNASVGNNLSTQVSGAYIFRPNQEKPLMVSHWAQTRLVKTPLVQEVH |
| | | QNFSAWCSQVVR |
| | | LYRGQRHLELEWTVGPIPVGDGWGKEIISRFDTVLETKGLFYTDSNGREILERRRDYRPT |
| | | WKLNQTETVA |
| | | GNYYPVNSRIYIRDGNMQLTVLTDRSQGGSSLRDGSMELMVHRRLLKDDGRGVGEALL |
| | | EDGLGRWVRGRH |
| | | LVLLDKVRTAATGHRLQAEKEVLTPQVVLAPGGGAPYHLKVAPRKQFSGLRRELPPSVH |
| | | LLTLARWDQKT |
| | | LLLRLEHQFAVGEDSGNLSSPVTLDLTDLFSAFTITYLQETTLVANQLRASASRLKWTPN |
| | | TGPTPLPSPS |
| | | RLDPATITLQPMEIRTFLASVQWEEHG |
| XP_419762.5 | SEQ ID NO: 150 | MPAASLLPLFGSAAGPGALGGPAGGGAGGGGRKAAGPGAFRLTEKFVLLLVFSAFITLC |
| PREDICTED: | | FGAIFFLPDSS |
| mannosyl- | | KLLSGVFFHSAALQPPPPPPGFQPRAPPQPGAGPAMPEEAGGAGSLERIRADHERALREA |
| oligosaccharide | | KETLQKLPEE |
| 1,2-alpha- | | IRRDIRQDKEKLLQDARGRKEAAAAGLPQRPFRQPVGAVGREPADLAVRQRRDKIKEM |
| mannosidase IA | | MKYAWDNYKRYA |
| [Gallus gallus] | | WGLNELKPISKQGHSSNLFGNIQGATIVDALDTLFIMEMKEEFKEAKEWVEKNLDFNVN |
| | | AEISVFEVNIR |
| | | FVGGLLSAYYLSGEEIFRKKAVELGEKLLPAFNTPTGIPWALLNIKSGIGRNWPWASGGS |
| | | SILAEFGTLH |
| | | LEFVHLSHLSGNPVFAEKVMNIRKVLSRLDKPEGLYPNYLNPSSGQWGQHHVSIGGLGD |
| | | SFYEYLLKAWL |
| | | MSDKTDEEGKKMYYDAVQAIETHLIRKSSGGLTYIAEWKGGLLEHKMGHLTCFAGGMF |
| | | ALGADGAPSDKT |
| | | GHHIELGAEIARTCHESYDRTSMKLGPEAFRFDGGVEAIATRQNEKYYILRPEVIETYMY |
| | | MWRLTHDPKY |
| | | RQWAWEAVEALEKHCRVDGGYSGIRDVYSNHESHDDVQQSFFLSETLKYLYLLFSDDD |
| | | LLPFEHWVFNTE |
| | | AHPFPILRKEDGSKEEKEK |
| NoManIB | SEQ ID NO: 153 | MARRRYRLFMICAAVILFLLYRVSQNTWDDSAHYATLRHPPASNPPAAGGESPLKPAAK |
| | | PEHEHEHENGYAPESKPKPQSEPKPESKPAPEHAAGGQKSQGKPSYEDDEETGKNPPKSA |
| | | VIPSDTRLPPDNKVHWRPVKEHFPVPSESVISLPTGKPLKVPRVQHEFGVESPEAKSRRVA |
| | | RQERVGKEIERAWSGYKKFAWMHDELSPVSAKHRDPFCGWAATLVDSLDTLWIAGLKE |
| | | QFDEAARAVEQIDFTTTPRNNIPVFETTIRYLGGLLGAFDVSGGHDGGYPMLLTKAVELA |
| | | EILMGIFDTPNRMPILYYQWQPEYASQPHRAGSVGIAELGTLSMEFTRLAQLTSQYKYYD |
| | | AVDRITDALIELQKQGTSIPGLFPENLDASGCNHTATALRSSLSEAAQKQMDEDLSNKPE |
| | | NYRPGKNSKADPQTVEKQPAKKQNEPVEKAKQVPTQQTAKRGKPPFGANGFTANWDC |
| | | VPQGLVVGGYGFQQYHMGGGQDSAYEYFPKEYLLLGGLESKYQKLYVDAVEAINEWL |
| | | LYRPMTDGDWDILFPAKVSTAGNPSQDLVATFEVTHLTCFIGGMYGLGGKIFGREKDLE |
| | | TAKRLTDGCVWAYQSTVSGIMPEGSQVLACPTLEKCDFNETLWWEKLDPAKDWRDKQ |
| | | VADDKDKATVGEALKETANSHDAAGGSKAVHKRAAVPLPKPGADDDVGSELPQSLKD |
| | | KIGFKNGEQKKPTGSSVGIQRDPDAPVDSVLEAHRLPPQEPEEQQVILPDKPQTHEEFVK |
| | | QRIAEMGFAPGVVHIQSRQYILRPEAIESVWYMYRITGDPIWMEKGWKMFEATIRATRTE |
| | | IANSAIDDVNSEEPGLKDEMESFWLAETLKYYYLLFSEPSVISLDEWVLNTEAHPFKRPG |
| | | GSVIGHSI |
| cDNA sequence of | SEQ ID NO: 152 | ATG CCA GCT GCT TCT TTG TTG CCA TTG TTT GGT TCT GCT GCT GGT CCA GGT G |
| Gallus gallus | | CT TTG GGT GGT CCA GCT GGT GGT GGT GCT GGT GGT GGT GGT AGA AAGGCT G |
| protein sequence | | CT GGT CCA GGT GCT TTT AGA TTG ACT GAA AAG TTT GTT TTG TTG TTG GTT TT |
| chosen for | | T TCT GCT TTT ATT ACT TTG TGT TTT GGT GCT ATT TTT TTT TTGCCA GAT TCT |
| expression | | TCT AAG TTG TTG TCT GGT GTT TTT TTT CAT TCT GCT GCT TTG CAA CCA CCA |
| | | CCA CCA CCA CCA GGT TTT CAA CCA AGA GCT CCA CCA CAACCA GGT GCT GGT CCA |
| | | GCT ATG CCA GAA GAA GCT GGT GGT GCT GGT TCT TTG GAA AGA ATT AGA GCT |
| | | GAT CAT GAA AGA GCT TTG AGA GAA GCT AAG GAAACT TTG CAA AAG TTG CCA |
| | | GAA GAA ATT AGA AGA GAT ATT AGA CAA GAT AAG GAA AAG TTG TTG CAA GA |
| | | T GCT AGA GGT AGA AAG GAA GCT GCT GCT GCTGGT TTG CCA CAA AGA CCA TT |
| | | T AGA CAA CCA GTT GGT GCT GTT GGT AGA GAA CCA GCT GAT TTG GCT GTT AG |
| | | A CAA AGA AGA GAT AAG ATT AAG GAA ATGATG AAG TAG GCT TGG GAT AAC T |
| | | AC AAG AGA TAC GCT TGG GGT TTG AAC GAA TTG AAG CCA ATT TCT AAG CAA |
| | | GGT CAT TCT TCT AAC TTG TTT GGT AACATT CAA GGT GCT ACT ATT GTT GAT G |
| | | CT TTG GAT ACT TTG TTT ATT ATG GAA ATG AAG GAA GAA TTT AAG GAA GCT A |
| | | AG GAA TGG GTT GAA AAG AAC TTGGAT TTT AAC GTT AAC GCT GAA ATT TCT G |
| | | TT TTT GAA GTT AAC ATT AGA TTT GTT GGT GGT TTG TTG TCT GCT TAC TAC TTG |
| | | TCT GGT GAA GAA ATT TTTAGA AAG AAG GCT GTT GAA TTG GGT GAA AAG TTG |
| | | TTG CCA GCT TTT AAC ACT CCA ACT GGT ATT CCA TGG GCT TTG TTG AAC ATT A |
| | | AG TCT GGT ATT GGTAGA AAC TGG CCA TGG GCT TCT GGT GGT TCT TCT ATT TT |
| | | G GCT GAA TTT GGT ACT TTG CAT TTG GAA TTT GTT CAT TTG TCT CAT TTG TCT |
| | | GGT AAC CCAGTT TTT GCT GAA AAG GTT ATG AAC ATT AGA AAG GTT TTG TCT A |
| | | GA TTG GAT AAG CCA GAA GGT TTG TAC CCA AAC TAC TTG AAC CCA TCT TCT G |
| | | GT CAATGG GGT CAA CAT CAT GTT TCT ATT GGT GGT TTG GGT GAT TCT TTT TA |
| | | C GAA TAC TTG TTG AAG GCT TGG TTG ATG TCT GAT AAG ACT GAT GAA GAA GG |
| | | TAAG AAG ATG TAC TAC GAT GCT GTT CAA GCT ATT GAA ACT CAT TTG ATT AG |
| | | A AAG TCT TCT GGT GGT TTG ACT TAC ATT GCT GAA TGG AAG GGT GGT TTGTTG |
| | | GAA CAT AAG ATG GGT CAT TTG ACT TGT TTT GCT GGT GGT ATG TTT GCT TTG |
| | | GGT GCT GAT GGT GCT CCA TCT GAT AAG ACT GGT CAT CAT ATT GAATTG GGT G |
| | | CT GAA ATT GCT AGA ACT TGT CAT GAA TCT TAC GAT AGA ACT TCT ATG AAG T |
| | | TG GGT CCA GAA GCT TTT AGA TTT GAT GGT GGT GTT GAA GCTATT GCT ACT AG |
| | | A CAA AAC GAA AAG TAC TAC ATT TTG AGA CCA GAA GTT ATT GAA ACT TAC AT |
| | | G TAC ATG TGG AGA TTG ACT CAT GAT CCA AAG TAC AGACAA TGG GCT TGG GA |
| | | A GCT GTT GAA GCT TTG GAA AAG CAT TGT AGA GTT GAT GGT GGT TAC TCT GG |
| | | T ATT AGA GAT GTT TAC TCT AAC CAT GAA TCT CATGAT GAT GTT CAA CAA TCT |
| | | TTT TTT TTG TCT GAA ACT TTG AAG TAC TTG TAC TTG TTG TTT TCT GAT GAT GA |
| | | T TTG TTG CCA TTT GAA CAT TGG GTT TTTAAC ACT GAA GCT CAT CCA TTT CCA |
| | | ATT TTG AGA AAG GAA GAT GGT TCT AAG GAA GAA AAG GAA AAG |
| Codon optimized | SEQ ID NO: 153 | ATG CCA GCA GCA TCC TTA CTT CCA TTA TTT GGC TCC GCA GCT GCA CCT GGC |
| Gallus gallus | | GCT TTA GGT GGT CCT GCT GGC GGC GGA GCC GGA GGC GGC GGC CGT AAAGCC |
| cDNA | | GCA GGT CCT GGT GCA TTC AGG CTG ACC GAG AAA TTC GTC CTG CTA CTT GTC |
| | | TTT TCA GCT TTT ATA ACG CTG TGT TTC GGC GCA ATT TTT TTT CTTCCT GAT TC |
| | | C TCC AAA CTT CTT TCA GGT GTC TTT TTC CAT AGT GCA GCA CTT CAA CCT CCT |
| | | CCC CCC CCT CCA GGT TTC CAA CCC AGA GCT CCT CCA CAACCA GGA GCT GGA |
| | | CCT GCC ATG CCC GAA GAG GCA GGA GGT GCC GGT AGT CTA GAA AGA ATA AG |
| | | G GCA GAC CAC GAA AGA GCA CTT CGT GAG GCT AAA GAAACC CTA CAG AAA C |
| | | TT CCC GAG GAG ATC CGT AGG GAC ATA AGG CAA GAT AAA GAA AAA CTT TTA |
| | | CAA GAC GCA CGT GGT CGT AAA GAA GCC GCC GCC GCAGGA CTA CCC CAA AGA |
| | | CCA TTT CGT CAG CCT GTT GGC GCT GTC GGA AGG GAA CCC GCT GAT CTT GCA |
| | | GTA AGA CAG AGA AGA GAC AAA ATC AAG GAG ATGATG AAG TAT GCC TGG GA |
| | | C AAT TAT AAG CGT TAT GCC TGG GGA CTA AAT GAG CTA AAA CCT ATT TCT AA |
| | | A CAG GGA CAC ACT TCT AAT TTA TTT GGA AACATC CAA GGT GCC ACC ATA GT |
| | | T GAT GCA CTT GAT ACT CTG TTC ATA ATG GAG ATG AAA GAA GAG TTC AAA GA |
| | | G GCA AAA GAA TGG GTA GAG AAA AAC CTTGAT TTC AAC GTA AAC GCA GAA A |
| | | TC AGT GTC TTC GAA GTA AAT ATA AGA TTC GTT GGA GGC CTA CTT TCC GCT TA |
| | | T TAT TTA TCA GGA GAG GAA ATA TTTCGT AAG AAG GCC GTG GAA TTA GGT GA |
| | | A AAA CTT TTG CCA GCT TTT AAC ACC CCA ACA GGA ATT CCT TGG GCT TTG TTG |
| | | AAT ATC AAG AGT GGA ATC GGTAGA AAC TGG CCT TGG GCT TCT GGT GGA AGT |
| | | TCA ATA TTG GCC GAA TTT GGA ACT CTT CAT TTA GAA TTC GTC CAT TTA TCC C |
| | | AT CTA AGT GGT AAC CCAGTT TTC GCC GAG AAA GTA ATG AAT ATT CGT AAA G |
| | | TT TTG TCT CGT CTT GAT AAG CCT GAG GGC CTG TAC CCT AAC TAG CTT AAT CC |
| | | C TCT TCA GGC CAATGG GGC CAG CAC CAC GTG TCC ATC GGC GGT CTT GGA GA |
| | | T AGT TTT TAT GAG TAT CTG CTG AAG GCT TGG TTA ATG TCC GAC AAG ACT GA |
| | | C GAA GAG GGCAAA AAG ATG TAT TAT GAT GCC GTC CAA GCT ATC GAG ACT CA |
| | | C TTA ATT AGG AAG TCT AGT GGT GGT CTG ACC TAT ATA GCC GAA TGG AAG GG |
| | | C GGC CTTCTT GAA CAC AAA ATG GGT CAC TTA ACC TGC TTT GCA GGA GGT AT |
| | | G TTT GCT TTA GGC GCA GAC GGC GCC CCC TCA GAT AAA ACG GGA CAT CAT AT |
| | | T GAGTTA GGA GCC GAG ATT GCC AGG ACA TGC CAC GAA TCA TAT GAT AGG AC |
| | | G AGT ATG AAG TTA GGT CCT GAG GCA TTC AGA TTT GAT GGC GGC GTT GAG GC |
| | | AATC GCT ACC AGA CAA AAT GAG AAA TAC TAC ATT TTA AGA CCA GAA GTC AT |
| | | T GAG ACC TAC ATG TAC ATG TGG CGT CTA ACT CAT GAC CCC AAA TAT CGTCA |
| | | G TGG GCA TGG GAG GCC GTT GAA GCC CTA GAA AAA CAT TGC AGA GTT GAC G |
| | | GC GGT TAT AGT GGC ATA CGT GAT GTC TAT TCA AAC CAT GAG TCC CACGAC G |
| | | AC GTA CAA CAG TCT TTT TTT CTT TCA GAG ACA CTT AAG TAC CTA TAC CTA CT |
| | | A TTC AGT GAC GAC GAT CTT CTA CCT TTC GAA CAT TGG GTT TTCAAC ACC GAA |
| | | GCT CAT CCC TTC CCC ATC TTA CGT AAG GAG GAC GGT TCC AAA GAG GAA AAA |
| | | GAG AAA |
| Homo sapiens | SEQ ID NO: 154 | MRFPSIFTAVLFAASSALAAPVNTTTEDETAQIPAEAVIGYSDLEGDFDVAVLPFSNSTNN |
| ORM1; HsORM1; | | GLLFINTTIASIAAKEEGVSLDKREAEAQIPLCANLVPVPITNATLDQITGKWFYIASAF |
| uniport P02763 | | RNEEYNKSVQEIQATFFYFIPNKTEDTIFLREYQTRQDQCIYNTTYLNVQRENGTIS |
| | | RYVGGQEHFAHLLILRDTKTYMLAFDVNDEKNWGLSVYADKPETTKEQLGEFYEA |
| | | LDCLRIPKSDVVYTDWKKDKCEPLEKQHEKERKQEEGES* |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-41. (canceled)

42. A method of producing a consumable composition comprising:
a. recombinantly expressing a nutritional protein in a host cell, wherein the nutritional protein is secreted from of the host cell;
b. recombinantly expressing an α-1,2-mannosidase in the host cell;
wherein the α-1,2-mannosidase reduces the glycosylation of greater than 50% of the nutritional protein secreted from the host cell and, wherein the nutritional protein with reduced glycosylation is mixed with at least one more component to form the consumable composition.

43. The method of claim 42, wherein the α-1,2-mannosidase has a sequence of SEQ ID No: 7, a functional equivalent thereof or a sequence 85% or more identical to SEQ ID No: 7.

44. The method of claim 42, wherein the α-1,2-mannosidase has a sequence of SEQ ID No: 150, a functional equivalent thereof or a sequence 85% or more identical to SEQ ID No: 150.

45. The method of claim 42, wherein the nutritional content of the consumable composition is equal to or greater than the nutritional content of a control composition wherein the control composition is produced using the same protein isolated from a native source or the recombinant nutritional protein un-modified by the α-1,2-mannosidase.

46. The method of claim 45, wherein the nutritional content is a protein content of the composition.

47. The method of claim 46, wherein the protein content of the consumable composition is at least 5%, at least 10% or at least 20% higher than the control composition.

48. The method of claim 42, wherein at least 75% of the nutritional protein secreted from the host cell has reduced glycosylation as compared to a control protein wherein the control protein is isolated from a native source or is the recombinant nutritional protein un-modified by the α-1,2-mannosidase.

49. The method of claim 48, wherein at least 80% of the nutritional protein secreted from the host cell has reduced glycosylation as compared to the control protein.

50. The method of claim 49, wherein at least 90% of the nutritional protein secreted from the host cell has reduced glycosylation as compared to the control protein.

51. The method of claim 42, wherein a thermal stability of the nutritional protein is increased as compared to a control composition wherein the control composition is produced using the same protein isolated from a native source or the recombinant nutritional protein un-modified by the α-1,2-mannosidase.

52. The method of claim 42, wherein the host cell is Pichia pastoris.

53. The method of claim 42, wherein the nitrogen to carbon ratio of the nutritional protein is equal to or greater than the ratio of the nutritional protein isolated from its native source.

54. The method of claim 42, wherein the nutritional protein is an animal or avian protein.

55. A consumable composition produced using the method of claim 42

56. The consumable composition of claim 55, wherein the composition is a beverage.

57. The consumable composition of claim 55, wherein the composition is a foodstuff.

58. A host cell used for the expression of a recombinant nutritional protein comprising:
c. a first promoter driving expression of a nutritional protein;
d. a second promoter driving expression of an α-1,2-mannosidase with sequence of SEQ ID Nos: 7 or 150, a functional equivalent thereof or a sequence 85% or more identical to SEQ ID Nos: 7 or 150;
wherein the mannosylation of the nutritional protein is reduced as a result of the expression of the α-1,2-mannosidase.

59. The host cell of claim 58, wherein the host cell is Pichia pastoris.

60. The host cell of claim 58, wherein the nutritional protein and the α-1,2-mannosidase are expressed using one or more expression cassettes.

61. The host cell of claim 58, wherein the nutritional protein and the α-1,2-mannosidase are expressed on separate expression constructs.


SEQUENCE LISTING (156 sequences)

SEQ ID NO: 1 - protein, 588 aa, Trichoderma reesei
MRFPSIFTAVLFAASSALAAPVNTTTEDETAQIPAEAVIGYSDLEGDFDVAVLPFSNSTNNGLLFINTTIASIAAKEEGVSLEKREAEAATKRGSPNPTRAAAVKAAFQTSWNAYHHFAFPHDDLHPVSNSFDDERNGWGSSAIDGLDTAILMGDADIVNTILQYVPQINFTTTAVANQGSSVFETNIRYLGGLLSAYDLLRGPFSSLATNQTLVNSLLRQAQTLANGLKVAFTTPSGVPDPTVFFNPTVRRSGASSNNVAEIGSLVLEWTRLSDLTGNPQYAQLAQKGESYLLNPKGSPEAWPGLIGTFVSTSNGTFQDSSGSWSGLMDSFYEYLIKMYLYDPVAFAHYKDRWVLGADSTIGHLGSHPSTRKDLTFLSSYNGQSTSPNSGHLASFGGGNFILGGILLNEQKYIDFGIKLASSYFGTYTQTASGIGPEGFAWVDSVTGAGGSPPSSQSGFYSSAGFWVTAPYYILRPETLESLYYAYRVTGDSKWQDLAWEALSAIEDACRAGSAYSSINDVTQANGGGASDDMESFWFAEALKYAYLIFAEESDVQVQATGGNKFVFNTEAHPFSIRSSSRRGGHLA

SEQ ID NO: 2 - protein, 628 aa, Gallus gallus
MVLPRKLPGMPGWPAALGLRLPQKFLFLLFLSGLLTLCFGALFLLPDSSRFKRLFLPRRATSSSSSSSSSSTRDTELPRSPPAAAEPRHASPAAPRRLREKLRARNAAPAAHTAPASRPQGPDGERPAEVGTGAPRESRAPFHFDYERFRQSLRHPVRGGRPDQDPDTRARKMKIKEMMKFAWDNYKQYALGKNELRPLTKNGHIGNMFGGLRGATVVDALDTLYIMELEEEFQEAKTWVEKSFDLNVNGEASLFEVNIRYIGGLLAAYYLTGEEVFKSKALELGEKLLPAFNTPTGIPRGVINLGSGMSWSWGWASAGSSILAEFGTLHLEFLHLSELSGNPVFAEKVLNIRKVLKRVEKPQGLYPNFLSPVTGNWVQHHVSIGGLGDSFYEYLIKSWLMSDKKDSEAKKMYDDALEAIEKHLVKKSAGGLTYIAEWRGGILDHKMGHLACFSGGMIALGAEHGGEERKQHYMDLAAEITNTCHESYARSDTKLGPEAFRFDAGTEAMATRLSERYYILRPEVVESYVYMWRLTHDVKYRQWGWEVVKALEKHCRVEAGFSGIRDVYTTVPTHDNMQQSFFLAETLKYLYLLFCEDDVLSLDDWVFNTEAHPLPVNHSNFKAKASVQ

SEQ ID NO: 3 - protein, 560 aa, Trichoderma reesei
MRCSLFLRLHYESYFWTTLPTNYPPKQIRPLPTTSPLKFPKIQAASPSELPEALKTRLQRQTAVKDVFSKCWASYKRHAWKADELAPVSGGQKNPFGGWAATLVDSLDTLYLMDMKPEFDEAVAAAASIDFTKTDLDEVNVFETTIRYLGGFLSAYDLSADARLLSKAVEVGEMLYHAFDTPNRMPITRWAIHAAMAGKKQVAPAGLLVAEIGSLSMEFTRLSMLTRDPKWFDAVQRITEGMAAQQNATALPGLWPLVVSAQDEIYSVGDTFTLGAMADSVYEYLPKMSALTGGQLPVYREMYEAAMATALKHNLFRPMTPSNQDILVAGTVKADGGVKTTLEPQGQHLVCFLGGLLTLGGKLFGRQQDLDAARRLVDGCVWTYKALPRGIMPETFFMLPCPSSTCAWDEASWKRGVLARAAKDAADKASDDDDADAIISRDRLPKGFTSIPDRRYILRPEAIESVFVSYRATAEPSLMESAWDMFTAINATTSTRLANSAVWDVTRPMGEDPGMADSMESFWMGETLKYFYLVFAAWDDVSLDEWVFNTEAHPFRRLLP

SEQ ID NO: 4 - protein, 570 aa, Trichoderma reesei
MLNQLQGRVPRRYIALVAFAFFVAFLLWSGYDFVPRTATVGRFKYVPSSYDWSKAKVYYPVKDMKTLPQGTPVTFPRLQLRNQSEAQDDTTKARKQAVKDAFVKSWEAYKTYAWTKDQLQPLSLSGKETFSGWSAQLVDALDTLWIMDLKDDFFLAVKEVAVIDWSKTKDNKVINLFEVTIRYLGGLIAAYDLSQEPVLRAKAIELGDTLYATFDTPNRLPSHWLDYSKAKKGTQRADDSMSGAAGGTLCMEFTRLSQITGDPKYYDATERIKQFFYRFQNETTLPGMWPVMMNYREETMVESRYSMGGSADSLYEYLVKMPALLGGLDPQYPEMAIRALDTARDNLLFRPMTEKGDNILALGNALVDHGNVQRITEMQHLTCFAGGMYAMAGKLFKRDDYVDLGSRISSGCVWAYDSFPSGIMPESADMAACAKLDGPCPYDEVKAPVDPDGRRPHGFIHVKSRHYLLRPEAIESVFYMWRITGDQVWRDTAWRMWENIVREAETEHAFAIVEDVTRTASKLTNNYLLQTFWLAETLKYFYLIFDDESAIDLDKWVFNTEAHPFKRPAV

SEQ ID NO: 5 - protein, 1070 aa, Trichoderma reesei
MVMLVAIALAWLGCSLLRPVDAMRADYLAQLRQETVDMFYHGYSNYMEHAFPEDELRPISCTPLTRDRDNPGRISLNDALGNYSLTLIDSLSTLAILAGGPQNGPYTGPQALSDFQDGVAEFVRHYGDGRSGPSGAGIRARGFDLDSKVQVFETVIRGVGGLLSAHLFAIGELPITGYVPRPEGVAGDDPLELAPIPWPNGFRYDGQLLRLALDLSERLLPAFYTPTGIPYPRVNLRSGIPFYVNSPLHQNLGEAVEEQSGRPEITETCSAGAGSLVLEFTVLSRLTGDARFEQAAKRAFWEVWHRRSEIGLIGNGIDAERGLWIGPHAGIGAGMDSFFEYALKSHILLSGLGMPNASTSRRQSTTSWLDPNSLHPPLPPEMHTSDAFLQAWHQAHASVKRYLYTDRSHFPYYSNNHRATGQPYAMWIDSLGAFYPGLLALAGEVEEAIEANLVYTALWTRYSALPERWSVREGNVEAGIGWWPGRPEFIESTYHIYRATRDPWYLHVGEMVLRDIRRRCYAECGWAGLQDVQTGEKQDRMESFFLGETAKYMYLLFDPDHPLNKLDAAYVFTTEGHPLIIPKSKRGSGSHNRQDRARKAKKSRDVAVYTYYDESFTNSCPAPRPPSEHHLIGSATAARPDLFSVSRFTDLYRTPNVHGPLEKVEMRDKKKGRVVRYRATSNHTIFPWTLPPAMLPENGTCAAPPERIISLIEFPANDITSGITSRFGNHLSWQTHLGPTVNILEGLRLQLEQVSDPATGEDKWRITHIGNTQLGRHETVFFHAEHVRHLKDEVFSCRRRRDAVEIELLVDKPSDTNNNNTLASSDDDVVVDAKAEEQDGMLADDDGDTLNAETLSSNSLFQSLLRAVSSVFEPVYTAIPESDPSAGTAKVYSFDAYTSTGPGAYPMPSISDTPIPGNPFYNFRNPASNFPWSTVFLAGQACEGPLPASAPREHQVIVMLRGGCSFSRKLDNIPSFSPHDRALQLVVVLDEPPPPPPPPPANDRRDVTRPLLDTEQTTPKGMKRLHGIPMVLVRAARGDYELFGHAIGVGMRRKYRVESQGLVVENAVVL

SEQ ID NO: 6 - protein, 523 aa, Trichoderma reesei
MRFPSSSVLALGLIGPALAYPKPGATKRGSPNPTRAAAVKAAFQTSWNAYHHFAFPHDDLHPVSNSFDDERNGWGSSAIDGLDTAILMGDADIVNTILQYVPQINFTTTAVANQGISVFETNIRYLGGLLSAYDLLRGPFSSLATNQTLVNSLLRQAQTLANGLKVAFTTPSGVPDPTVFFNPTVRRSGASSNNVAEIGSLVLEWTRLSDLTGNPQYAQLAQKGESYLLNPKGSPEAWPGLIGTFVSTSNGTFQDSSGSWSGLMDSFYEYLIKMYLYDPVAFAHYKDRWVLAADSTIAHLASHPSTRKDLTFLSSYNGQSTSPNSGHLASFAGGNFILGGILLNEQKYIDFGIKLASSYFATYNQTASGIGPEGFAWVDSVTGAGGSPPSSQSGFYSSAGFWVTAPYYILRPETLESLYYAYRVTGDSKWQDLAWEAFSAIEDACRAGSAYSSINDVTQANGGGASDDMESFWFAEALKYAYLIFAEESDVQVQANGGNKFVFNTEAHPFSIRSSSRRGGHLA

SEQ ID NO: 7 - protein, 893 aa, Trichoderma reesei
MARRRYRLFMICAAVILFLLYRVSQNTWDDSAHYATLRHPPASNPPAAGGESPLKPAAKPEHEHEHENGYAPESKPKPQSEPKPESKPAPEHAAGGQKSQGKPSYEDDEETGKNPPKSAVIPSDTRLPPDNKVHWRPVKEHFPVPSESVISLPTGKPLKVPRVQHEFGVESPEAKSRRVARQERVGKEIERAWSGYKKFAWMHDELSPVSAKHRDPFCGWAATLVDSLDTLWIAGLKEQFDEAARAVEQIDFTTTPRNNIPVFETTIRYLGGLLGAFDVSGGHDGGYPMLLTKAVELAEILMGIFDTPNRMPILYYQWQPEYASQPHRAGSVGIAELGTLSMEFTRLAQLTSQYKYYDAVDRITDALIELQKQGTSIPGLFPENLDASGCNHTATALRSSLSEAAQKQMDEDLSNKPENYRPGKNSKADPQTVEKQPAKKQNEPVEKAKQVPTQQTAKRGKPPFGANGFTANWDCVPQGLVVGGYGFQQYHMGGGQDSAYEYFPKEYLLLGGLESKYQKLYVDAVEAINEWLLYRPMTDGDWDILFPAKVSTAGNPSQDLVATFEVTHLTCFIGGMYGLGGKIFGREKDLETAKRLTDGCVWAYQSTVSGIMPEGSQVLACPTLEKCDFNETLWWEKLDPAKDWRDKQVADDKDKATVGEALKETANSHDAAGGSKAVHKRAAVPLPKPGADDDVGSELPQSLKDKIGFKNGEQKKPTGSSVGIQRDPDAPVDSVLEAHRLPPQEPEEQQVILPDKPQTHEEFVKQRIAEMGFAPGVVHIQSRQYILRPEAIESVWYMYRITGDPIWMEKGWKMFEATIRATRTEIANSAIDDVNSEEPGLKDEMESFWLAETLKYYYLLFSEPSVISLDEWVLNTEAHPFKRPGGSVIGHSI

SEQ ID NO: 8 - protein, 523 aa, Trichoderma reesei; identical to SEQ ID NO: 6

SEQ ID NO: 9 - protein, 523 aa, Trichoderma reesei
MRFPSSSVLALGLIGPALAYPKPGATKRGSPNPTRAAAVKAAFQTSWNAYHHFAFPHDDLHPVSNSFDDERNGWGSSAIDGLDTAILMGDADIVNTILQYVPQINFTTTAVANQGSSVFETNIRYLGGLLSAYDLLRGPFSSLATNQTLVNSLLRQAQTLANGLKVAFTTPSGVPDPTVFFNPTVRRSGASSNNVAEIGSLVLEWTRLSDLTGNPQYAQLAQKGESYLLNPKGSPEAWPGLIGTFVSTSNGTFQDSSGSWSGLMDSFYEYLIKMYLYDPVAFAHYKDRWVLGADSTIGHLGSHPSTRKDLTFLSSYNGQSTSPNSGHLASFGGGNFILGGILLNEQKYIDFGIKLASSYFGTYTQTASGIGPEGFAWVDSVTGAGGSPPSSQSGFYSSAGFWVTAPYYILRPETLESLYYAYRVTGDSKWQDLAWEALSAIEDACRAGSAYSSINDVTQANGGGASDDMESFWFAEALKYAYLIFAEESDVQVQATGGNKFVFNTEAHPFSIRSSSRRGGHLA

SEQ ID NO: 10 - protein, 588 aa, Trichoderma reesei; identical to SEQ ID NO: 1

SEQ ID NO: 11 - protein, 2087 aa, Gallus gallus
KEPVQIVQVSTVGRSECTTWGNFHFHTFDHVKFTFPGTCTYVFASHCNDSYQDFNIKIRRSDKNSHLIYFTVTTDGVILEVKETGITVNGNQIPLPFSLKSILIEDTCAYFQVTSKLGLTLKWNWADTLLLDLEETYKEKICGLCGNYDGNKKNDLILDGYKMHPRQFGNFHKVEDPSEKCPDVRPDDHTGRHPTEDDNRCSKYKKMCKKLLSRFGNCPKVVAFDDYVATCTEDMCNCVVNSSHSDLVSSCICSTLNQYSRDCVLSKGDPGEWRTKELCYQECPSNMEYMECGNSCADTCADPERSKICKAPCTDGCFCPPGTILDDLGGKKCVPRDSCPCMFQGKVYSSGGTYSTPCQNCTCKGGHWSCTSLPCSGSCSIDGGFHITTFDNKKFNFHGNCHYVLAKNTDDTFVVIGEIIQCGTSKTMTCLKNVLVTLGRTTIKICSCGSIYMNNFIVKLPVSKDGITIFRPSTFFIKILSSTGVQIRVQMKPVMQLSITVDHSYQNRTSGLCGNFNNIQTDDFRTATGAVEDSAAAFGNSWKTRASCFDVEDSFEDPCSNSVDKEKFAQHWCALLSNISSTFAACHSVVDPSVYIKRCMYDTCNAEKSEVALCSVLSTYSRDCAAAGMTLKGWRQGICDPSEECPETMVYNYSVKYCNQSCRSLDEPDPLCKVQIAPMEGCGCPEGTYLNDEEECVTPDDCPCYYKGKIVQPGNSFQEDKLLCKCIQGRLDCIGETVLVKDCPAPMYYFNCSSAGPGAIGSECQKSCKTQDMHCYVTECVSGCMCPDGLVLDGSGGCIPKDQCPCVHGGHFYKPGETIRVDCNTCTCNKRQWNCTDSPCKGTCTVYGNGHYMSFDGEKFDFLGDCDYILAQDFCPNNMDAGTFRIVIQNNACGKSLSICSLKITLIFESSEIRLLEGRIQEIATDPGAEKNYKVDLRGGYIVIETTQGMSFMWDQKTTVVVHVTPSFQGKVCGLCGDFDGRSRNDFTTRGQSVEMSIQEFGNSWKITSTCSNINMTDLCADQPFKSALGQKHCSIIKSSVFEACHSKVNPIPYYESCVSDFCGCDSVGDCECFCTSVAAYARSCSTAGVCINWRTPAICPVFCDYYNPPDKHEWFYKPCGAPCLKTCRNPQGKCGNILYSLEGCYPECSPDKPYFDEERRECVSLPDCTSCNPEEKLCTEDSKDCLCCYNGKTYPLNETIYSQTEGTKCGNAFCGPNGMIIETFIPCSTLSVPAQEQLMQPVTSAPLLSTEATPCFCTDNGQLIQMGENVSLPMNISGHCAYSICNASCQIELIWAECKVVQTEALETCEPNSEACPPTAAPNATSLVPATALAPMSDCLGLIPPRKFNESWDFGNCQIATCLGEENNIKLSSITCPPQQLKLCVNGFPFMKHHDETGCCEVFECQCICSGWGNEHYVTFDGTYYHFKENCTYVLVELIQPSSEKFWIHIDNYYCGAADGAICSMSLLIFHSNSLVILTQAKEHGKGTNLVLFNDKKVVPDISKNGIRITSSGLYIIVEIPELEVYVSYSRLAFYIKLPFGKYYNNTMGLCGTCTNQKSDDARKRNGEVTDSFKEMALDWKAPVSTNRYCNPGISEPVKIENYQHCEPSELCKIIWNLTECHRVVPPQPYYEACVASRCSQQHPSTECQSMQTYAALCGLHGICVDWRGQTNGQCEATCARDQVYKPCGEAKRNTCFSREVIVDTLLSRNNTPVFVEGCYCPDGNILLNEHDGICVSVCGCTAQDGSVKKPREAWEHDCQYCTCDEETLNISCFPRPCAKSPPINCTKEGFVRKIKPRLDDPCCTETVCECDIKTCIINKTACDLGFQPVVAISEDGCCPIFSCIPKGVCVSEGVEFKPGAVVPKSSCEDCVCTDEQDAVTGTNRIQCVPVKCQTTCQQGFRYVEKEGQCCSQCQQVACVANFPFGSVTIEVGKSYKAPYDNCTQYTCTESGGQFSLTSTVKVCLPFEESNCVPGTVDVTSDGCCKTCIDLPHKCKRSMKEQYIVHKHCKSAAPVPVPFCEGTCSTYSVYSFENNEMEHKCICCHEKKSHVEKVELVCSEHKTLKFSYVHVDECGCVETKCPMRRT

SEQ ID NO: 12 - protein, 186 aa, Gallus gallus
AEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 13 - protein, 186 aa, Gallus gallus
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 14 - protein, 186 aa, Gallus gallus
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYMNKCNACNAVVESNGTLTLSHFGKC

SEQ ID NO: 15 - protein, 426 aa, Gallus gallus
TRAPDCGGILTPLGLSYLAEVSKPHAEVVLRQDLMAQRASDLFLGSMEPSRNRITSVKVADLWLSVIPEAGLRLGIEVELRIAPLHAVPMPVRISIRADLHVDMGPDGNLQLLTSACRPTVQAQSTREAESKSSRSILDKVVDVDKLCLDVSKLLLFPNEQLMSLTALFPVTPNCQLQYLPLAAPVFSKQGIALSLQTTFQVAGAVVPVPVSPVPFSMPELASTSTSHLILALSEHFYTSLYFTLERAGAFNMTIPSMLTTATLAQKITQVGSLYHEDLPITLSAALRSSPRVVLEEGRAALKLFLTVHIGAGSPDFQSFLSVSADVTAGLQLSVSDTRMMISTAVIEDAELSLAASNVGLVRAALLEELFLAPVCQQVPAWMDDVLREGVHLPHLSHFTYTDVNVVVHKDYVLVPCKLKLRSTMA

SEQ ID NO: 16 - protein, 388 aa, Gallus gallus
MDSISVTNAKFCFDVFNEMKVHHVNENILYCPLSILTALAMVYLGARGNTESQMKKVLHFDSITGAGSTTDSQCGSSEYVHNLFKELLSEITRPNATYSLEIADKLYVDKTFSVLPEYLSCARKFYTGGVEEVNFKTAAEEARQLINSWVEKETNGQIKDLLVSSSIDFGTTMVFINTIYFKGIWKIAFNTEDTREMPFSMTKEESKPVQMMCMNNSFNVATLPAEKMKILELPYASGDLSMLVLLPDEVSGLERIEKTINFDKLREWTSTNAMAKKSMKVYLPRMKIEEKYNLTSILMALGMTDLFSRSANLTGISSVDNLMISDAVHGVFMEVNEEGTEATGSTGAIGNIKHSLELEEFRADHPFLFFIRYNPTNAILFFGRYWSP

SEQ ID NO: 17 - protein, 1185 aa, Gallus gallus
CSTWGGGHFSTFDKYQYDFTGTCNYIFATVCDESSPDFNIQFRRGLDKKIARIIIELGPSVIIVEKDSISVRSVGVIKLPYASNGIQIAPYGRSVRLVAKLMEMELVVMWNNEDYLMVLTEKKYMGKTCGMCGNYDGYELNDFVSEGKLLDTYKFAALQKMDDPSEICLSEEISIPAIPHKKYAVICSQLLNLVSPTCSVPKDGFVTRCQLDMQDCSEPGQKNCTCSTLSEYSRQCAMSHQVVFNWRTENFCSVGKCSANQIYEECGSPCIKTCSNPEYSCSSHCTYGCFCPEGTVLDDISKNRTCVHLEQCPCTLNGETYAPGDTMKAACRTCKCTMGQWNCKELPCPGRCSLEGGSFVTTFDSRSYRFHGVCTYILMKSSSLPHNGTLMAIYEKSGYSHSETSLSAIIYLSTKDKIVISQNELLTDDDELKRLPYKSGDITIFKQSSMFIQMHTEFGLELVVQTSPVFQAYVKVSAQFQGRTLGLCGNYNGDTTDDFMTSMDITEGTASLFVDSWRAGNCLPAMERETDPCALSQLNKISAETHCSILTKKGTVFETCHAVVNPTPFYKRCVYQACNYEETFPYICSALGSYARTCSSMGLILENWRNSMDNCTITCTGNQTFSYNTQACERTCLSLSNPTLECHPTDIPIEGCNCPKGMYLNHKNECVRKSHCPCYLEDRKYILPDQSTMTGGITCYCVNGRLSCTGKLQNPAESCKAPKKYISCSDSLENKYGATCAPTCQMLATGIECIPTKCESGCVCADGLYENLDGRCVPPEECPCEYGGLSYGKGEQIQTECEICTCRKGKWKCVQKSRCSSTCNLYGEGHITTFDGQRFVFDGNCEYILAMDGCNVNRPLSSFKIVTENVICGKSGVTCSRSISIYLGNLTIILRDETYSISGKNLQVKYNVKKNALHLMFDIIIPGKYNMTLIWNKHMNFFIKISRETQETICGLCGNYNGNMKDDFETRSKYVASNELEFVNSWKENPLCGDVYFVVDPCSKNPYRKAWAEKTCSIINSQVFSACHNKVNRMPYYEACVRDSCGCDIGGDCECMCDAIAVYAMACLDKGICIDWRTPEFCPVYCEYYNSHRKTGSGGAYSYGSSVNCTWHYRPCNCPNQYYKYVNIEGCYNCSHDEYFDYEKEKCMPCAMQPTSVTLPTATQPTSPSTSSASTVLTETTNPPV

SEQ ID NO: 18 - protein, 129 aa, Gallus gallus [sequence omitted]

SEQ ID NO: 19 - protein, 129 aa, Gallus gallus
KVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

SEQ ID NO: 20 - protein, 130 aa, Homo sapiens
KVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 21 - protein, 129 aa, Bos taurus
KVFERCELARTLKKLGLDGYKGVSLANWLCLTKWESSYNTKATNYNPSSESTDYGIFQINSKWWCNDGKTPNAVDGCHVSCRELMENDIAKAVACAKHIVSEQGITAWVAWKSHCRDHDVSSYVEGCTL

SEQ ID NO: 22 - protein, 449 aa, Gallus gallus
IEVNCSLYASGIGKDGTSWVACPRNLKPVCGTDGSTYSNECGICLYNREHGANVEKEYDGECRPKHVMIDCSPYLQVVRDGNTMVACPRILKPVCGSDSFTYDNECGICAYNAEHHTNISKLHDGECKLEIGSVDCSKYPSTVSKDGRTLVACPRILSPVCGTDGFTYDNECGICAHNAEQRTHVSKKHDGKCRQEIPEIDCDQYPTRKTTGGKLLVRCPRILLPVCGTDGFTYDNECGICAHNAQHGTEVKKSHDGRCKERSTPLDCTQYLSNTQNGEAITACPFILQEVCGTDGVTYSNDCSLCAHNIELGTSVAKKHDGRCREEVPELDCSKYKTSTLKDGRQVVACTMIYDPVCATNGVTYASECTLCAHNLEQRTNLGKRKNGRCEEDITKEHCREFQKVSPICTMEYVPHCGSDGVTYSNRCFFCNAYVQSNRTLNLVSMAAC

SEQ ID NO: 23 - protein, 139 aa, Gallus gallus
MAGARGCVVLLAAALMLVGAVLGSEDRSRLLGAPVPVDENDEGLQRALQFAMAEYNRASNDKYSSRVVRVISAKRQLVSGIKYILQVEIGRTTCPKSSGDLQSCEFHDEPEMAKYTTCTFVVYSIPWLNQIKLLESKCQ

SEQ ID NO: 24 - protein, 402 aa, Gallus gallus
MFFYNTDFRMGSISAANAEFCFDVFNELKVQHTNENILYSPLSIIVALAMVYMGARGNTEYQMEKALHFDSIAGLGGSTQTKVQKPKCGKSVNIHLLFKELLSDITASKANYSLRIANRLYAEKSRPILPIYLKCVKKLYRAGLETVNFKTASDQARQLINSWVEKQTEGQIKDLLVSSSTDLDTTLVLVNAIYFKGMWKTAFNAEDTREMPFHVTKEESKPVQMMCMNNSFNVATLPAEKMKILELPFASGDLSMLVLLPDEVSGLERIEKTINFEKLTEWTNPNTMEKRRVKVYLPQMKIEEKYNLTSVLMALGMTDLFIPSANLTGISSAESLKISQAVHGAFMELSEDGIEMAGSTGVIEDIKHSPELEQFRADHPFLFLIKHNPTNTIVYFGRYWSP

SEQ ID NO: 25 - protein, 388 aa, Gallus gallus; identical to SEQ ID NO: 16

SEQ ID NO: 26 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 27 - protein, 450 aa, Sus scrofa
SEVCFPRLGCFSDDAPWAGIVQRPLKILPWSPKDVDTRFLLYTNQNQNNYQELVADPSTITNSNFRMDRKTRFIIHGFIDKGEEDWLSNICKNLFKVESVNCICVDWKGGSRTGYTQASQNIRIVGAEVAYFVEVLKSSLGYSPSNVHVIGHSLGSHAAGEAGRRTNGTIERITGLDPAEPCFQGTPELVRLDPSDAKFVDVIHTDAAPIIPNLGFGMSQTVGHLDFFPNGGKQMPGCQKNILSQIVDIDGIWEGTRDFVACNHLRSYKYYADSILNPDGFAGFPCDSYNVFTANKCFPCPSEGCPQMGHYADRFPGKTNGVSQVFYLNTGDASNFARWRYKVSVTLSGKKVTGHILVSLFGNEGNSRQYEIYKGTLQPDNTHSDEFDSDVEVGDLQKVKFIWYNNNVINPTLPRVGASKITVERNDGKVYDFCSQETVREEVLLTLNPC

SEQ ID NO: 28 - protein, 455 aa, Capra hircus
GLVAADRITGGKDFRDIESKFALRTPEDTAEDTCHLIPGVTESVANCHFNHSSKTFVVIHGWTVTGMYESWVPKLVAALYKREPDSNVIVVDWLSRAQQHYPVSAGYTKLVGQDVAKFMNWMADEFNYPLGNVHLLGYSLGAHAAGIAGSLTSKKVNRITGLDPAGPNFEYAEAPSRLSPDDADFVDVLHTFTRGSPGRSIGIQKPVGHVDIYPNGGTFQPGCNIGEALRVIAERGLGDVDQLVKCSHERSVHLFIDSLLNEENPSKAYRCNSKEAFEKGLCLSCRKNRCNNMGYEINKVRAKRSSKMYLKTRSQMPYKVFHYQVKIHFSGTESNTYTNQAFEISLYGTVAESENIPFTLPEVSTNKTYSFLLYTEVDIGELLMLKLKWISDSYFSWSNWWSSPGFDIGKIRVKAGETQKKVIFCSREKMSYLQKGKSPVIFVKCHDKSLNRKSG

SEQ ID NO: 29 - protein, 685 aa, Sus scrofa
APKKGVRWCVISTAEYSKCRQWQSKIRRTNPMFCIRRASPTDCIRAIAAKRADAVTLDGGLVFEADQYKLRPVAAEIYGTEENPQTYYYAVAVVKKGFNFQLNQLQGRKSCHTGLGRSAGWNIPIGLLRRFLDWAGPPEPLQKAVAKFFSQSCVPCADGNAYPNLCQLCIGKGKDKCACSSQEPYFGYSGAFNCLHKGIGDVAFVKESTVFENLPQKADRDKYELLCPDNTRKPVEAFRECHLARVPSHAVVARSVNGKENSIWELLYQSQKKFGKSNPQEFQLFGSPGQQKDLLFRDATIGFLKIPSKIDSKLYLGLPYLTAIQGLRETAAEVEARQAKVVWCAVGPEELRKCRQWSSQSSQNLNCSLASTTEDCIVQVLKGEADAMSLDGGFIYTAGKCGLVPVLAENQKSRQSSSSDCVHRPTQGYFAVAVVRKANGGITWNSVRGTKSCHTAVDRTAGWNIPMGLLVNQTGSCKFDEFFSQSCAPGSQPGSNLCALCVGNDQGVDKCVPNSNERYYGYTGAFRCLAENAGDVAFVKDVTVLDNTNGQNTEEWARELRSDDFELLCLDGTRKPVTEAQNCHLAVAPSHAVVSRKEKAAQVEQVLLTEQAQFGRYGKDCPDKFCLFRSETKNLLFNDNTEVLAQLQGKTTYEKYLGSEYVTAIANLKQCSVSPLLEACAFMMR

SEQ ID NO: 30 - protein, 689 aa, Bos taurus
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 31 - DNA, 941 bp, Pichia pastoris
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattggatcccga

SEQ ID NO: 32 - DNA, 1048 bp, Pichia pastoris
aaatctgaacacgatgaaacctccccgtagattccaccgccccgttacttttttgggcaatcccgttgataagatccattttagagttgtttctgaaaggattacaggcgttgaagggtcagagagatgccagagaacagaccaattggtagtttgctaaagtggacgtctggcaggtgctctatcgtgttctttatttagggcgttacacttagtaggattacgtaacaatttggcttaaccttctaagttagaaagaaaccaagaggggtcctctttaacgttcagcagtatctaaaacacaaaacctgccctcataatacatcattctatctgtcaagctgtgctaccccacagaaatacccccaagagttaaagtgaaaagaaaagctaaatctgttagacttcaccccataacaaacttgatagttcctgtagccaatgaaagttaaccccattcaatgttccgagatctagtatgcttgctcctataaggaacgaagggttccagcttccttaccccatcaatggaaatctcctatttaccccccactggaaagatccgtccgaacgaacggataatagaaaaaagaaattcggacaaaatagaacacttatttagccaatgaaatccatttccagcatctccttcaactgccgttccatcccctttgttgagctacaccatcgtcagccagtaccgaataggaaacttaaccgatatcttggagaattctaatgcgcgaatgagtttagcctagatatccttagtgaagggttgttccgatacttctccacattcagtcatttcagatgggcagcattgttatcatgaagaaacggaaacgggcagtaagggttaaccgccaaattatataaagacaacatgtccccagtttaaagtttttctttcctattcttgtatcctgagtgaccgttgtgtttaaaataacaagttcgttttaacttaagaccaaaaccagttacaacaaattattccccaactaaacactaaagttcactcttatcaaactatcaaacatcaaag

SEQ ID NO: 33 - DNA, 991 bp, Pichia pastoris
cctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaagaattcgcg

SEQ ID NO: 34 - DNA, 1004 bp, Pichia pastoris
aaatcagccattaatctcacctcagtttttgaatcagtagaattttcaatgaaacaaacggttggtatattatttgatagggtagccaaatttccaaaaatgaacttttcatcaggtaatatcttgaataccgtaatgtagtgactattggaagaaactgctatcaaattatatttcggatagaaatccaaaccccagactgatctcttgagtctcaactctaagtcagccgcgactctaattatctgtggattaggagttagtgtggacaaagcatcagtatagtataactttacggttccattatcagacgctattgcaagaacttcctttccattgatctctccaattcgacagtaattgatatcataaggtaggtctggaaacacactggcgcttgtatcccattctgcaggaatttctggaacggtggtaatggtagttatccaacggagttggggtagttggtatatctggatatgccgcctataggataaaaacaggagagagtgaaccttgcttacggctactagattgttcttgtactcggaattgtcgttatcggaaactagactaatctcatctgtgtgttgcagtactattgagtcgttgtagtatctaccaggagggcattccatgaactagtgagacaaatgagttggattttctcaatagacatatgcaagaatgctacacaacggatgtcgcactctttttcttagttgataatatcatccaatcagaagacacgggctagaaggacttgctcccgaaggataatccactgctactatctcccttcctcacatatagtcttgcagggctcatgcccctttctccttcgaactgcccgatgaggaagtctttagcctatcaaggaattcgggaccatcatcaatttttagagccttacctgatcgcaatcaggatttcactactcatataaatacatcactcaaactccaactttgcttgttcatacaattcttgatattcacaggatc

SEQ ID NO: 35 - DNA, 260 bp, Pichia pastoris
aaattaaccagtgttttcttatctatttgtctttttacactaaagtgaagtacgaatccatgcgattgattcctcctcagatatcagctgaattcttgcttatgtaatacttgcgcgaactacatgtgaacttaggattcgataaggctggggggtcaaccaaccccacttcaaagagccgacccgtataaatagcctctgcgtcctcagatcaacaagacgaagcaatttttttttacctatcttcaggtgcctgttag

SEQ ID NO: 36 - DNA, 759 bp, Pichia pastoris
aaattctttttacgtggtgcgcatactggacagaggcagagtctcaatttcttcttttgagacaggctactacagcctgtgattcctcttggtacttggatttgcttttatctggctccgttgggaactgtgcctgggttttgaagtatcttgtggatgtgtttctaacactttttcaatcttcttggagtgagaatgcaggactttgaacatcgtctagctcgttggtaggtgaaccgttttaccttgcatgtggttaggagttttctggagtaaccaagaccgtcttatcatcgccgtaaaatcgctcttactgtcgctaataatcccgctggaagagaagttcgaacagaagtagcacgcaaagctcttgtcaaatgagaattgttaatcgtttgacaggtcacactcgtgggctatgtacgatcaacttgccggctgttgctggagagatgacaccagttgtggcatggccaattggtattcagccgtaccactgtatggaaaatgagattatcttgttcttgatctagtttcttgccattttagagttgccacattcgtaggtttcagtaccaataatggtaacttccaaacttccaacgcagataccagagatctgccgatccttccccaacaataggagcttactacgccatacatatagcctatctattttcactttcgcgtgggtgcttctatataaacggttccccatcttccgtttcatactacttgaattttaagcactaaagaatt

SEQ ID NO: 37 - DNA, 758 bp, Pichia pastoris
gtgaatttgtcacggaattgaccaagaggtcagacgatcctgtatcccattgagccgttatgctttgtgggggaaaccctatttctatcgtactaagaaaaccaatggtgaactcatattcggtatcaatggcgacgattccagcatagcctgtagacagtaacaacactagggcaacagcaactaacatatcttcattgatgaaacgttgtgatcggtgtgacttttatagtaaaagctacaactgtttgaaataccaagatatcattgtgaatggctcaaaagggtaatacatctgaaaaacctgaagtgtggaaaattccgatggagccaactcatgataacgcagaagtcccattttgccatcttctcttggtatgaaacggtagaaaatgatccgagtatgccaattgatactcttgattcatgccctatagtttgcgtagggtttaattgatctcctggtctatcgatctgggacgcaatgtagaccccattagtggaaacactgaaagggatccaacactctaggcggacccgctcacagtcatttcaggacaatcaccacaggaatcaactacttctcccagtcttccttgcgtgaagcttcaagcctacaacataacacttcttacttaatctttgattctcgaattgtttacccaatcttgacaacttagcctaagcaatactctggggttatatatagcaattgctcttcctcgctgtagcgttcattccatctttctagaattcgt

SEQ ID NO: 38 - DNA, 758 bp, Pichia pastoris
cttccccatttcactgacagtttgtagaaatagggcaacaattgatgcaaatcgattttcaacgcattggttttgatagcattgatgatcttggagctgtaaaagtccggctggataagctcaatgaaataggttggttgatctggatcttcttttgggtcattttgttcgctctgtatttcacaaattgccagaatctctgccaaccacagtggtaggtccaacttggtgttctgaatcacaggcttccccgggttgttctctaaataaccgaggcccggcacagaaatcgtaaaccgacacggtatcttttgtccgtccgccagtatctcatcaaggtcgtagtagcccatgatgagtatcaaaggggatttggttatgcgatgcaacgagagattgtttatcccagatgctgatgtaaaaaccttaaccagcgtgacagtagaaataagacacgttaaaattacccgcgcttccctaacaattggctctgcctttcggcaagtttctaactgccctcccctctcacatgcaccacgaacttaccgttcgctcctagcagaaccaccccaaagtttaatcaggaccgcattttagcctattgctgtagaaccccacaacataacctggtccagagccagccctttatatatggtaaatcccgtttgaacttcgaagtggaatcggaatttttacatcaaagaaactgatactgaaacttttggcttcgacttggactttctcttaatcgaattcgt

SEQ ID NO: 39 - DNA, 507 bp, Pichia pastoris
acacagttattattcatttaaatgtcaaaacagtagtgataaaaggctatgaaggaggttgtctaggggctcgcggaggaaagtgattcaaacagacctgccaaaaagagaaaaaagagggaatccctgttctttccaatggaaatgacgtaactttaacttgaaaaataccccaaccagaagggttcaaactcaacaaggattgcgtaattcctacaagtagcttagagctgggggagagacaactgaaggcagcttaacgataacgcggggggattggtgcacgactcgaaaggaggtatcttagtcttgtaacctcttttttccagaggctattcaagattcataggcgatatcgatgtggagaagggtgaacaatataaaaggctggagagatgtcaatgaagcagctggatagatttcaaattttctagatttcagagtaatcgcacaaaacgaaggaatcccaccaagacaaaaaaaaaaattctaaggaattccgaaacg

SEQ ID NO: 40 - DNA, 559 bp, Pichia pastoris
aaataagcatgtttgtttcagatcaaagattagcgtttcaaagttgtggaaaagtgaccatgcaacaatatgcaacacattcggattatctgataagtttcaaagctactaagtaagcccgtttcaagtctccagaccgacatctgccatccagtgattttcttagtcctgaaaaatacgatgtgtaaacataaaccacaaagatcggcctccgaggttgaacccttacgaaagagacatctggtagcgccaatgccaaaaaaaaatcacaccagaaggacaattcccttcccccccagcccattaaagcttaccatttcctattccaatacgttccatagagggcatcgctcggctcattttcgcgtgggtcatactagagcggctagctagtcggctgtttgagctctctaatcgaggggtaaggatgtctaatatgtcataatggctcactatataaagaacccgcttgctcaaccttcgactcctttcccgatcctttgcttgttgcttcttcttttataacaggaaacaaaggaatttatacactttaagaatt

SEQ ID NO: 41 - DNA, 798 bp, Pichia pastoris
caggtgaacccacctaactatttttaactggcatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgt

SEQ ID NO: 42 - DNA, 1009 bp, Pichia pastoris
aaataaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacagaatt

SEQ ID NO: 43 - DNA, 686 bp, Pichia pastoris
tgcttaagtaattgaaaacagtgttgtgattatataagcatggtatttgaatagaactactggggttaacttatctagtaggatggaagttgagggagatcaagatgcttaaagaaaaggattggccaatatgaaagccataattagcaatacttatttaatcagataattgtggggcattgtgacttgacttttaccaggacttcaaacctcaaccatttaaacagttatagaagacgtaccgtcacttttgcttttaatgtgatctaaatgtgatcacatgaactcaaactaaaatgatatcttttactggacaaaaatgttatcctgcaaacagaaagctttcttctattctaagaagaacatttacattggtgggaaacctgaaaacagaaaataaatactccccagtgaccctatgagcaggatttttgcatccctattgtaggcctttcaaactcacacctaatatttcccgccactcacactatcaatgatcacttcccagttctcttcttcccctattcgtaccatgcaacccttacacgccttttccatttcggttcggatgcgacttccagtctgtggggtacgtagcctattctcttagccggtatttaaacatacaaattcacccaaattctaccttgataaggtaattgattaatttcataaatgaattcgcg

SEQ ID NO: 44 - DNA, 477 bp, Pichia pastoris
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactat

SEQ ID NO: 45 - DNA, 1018 bp, Pichia pastoris
aaatagcagtttgcggtttcttgatttcatggggggaacaaacaatagtgttgccttaattctaattggcattgttgcttggaatcgaaattgggggataacgtcatatctgaaaagtaaacaacttcgggaaatcaggctgtttgaatggcttggaagcgagatagaaaggggatagcgagatagagggggcggagtagacgaagggtgttaaactgctgaaatctctcaatctggaagaaacggaataaattaactccttgcgataataaaatccgagtccgttatgaccccacaccgtgttgaccacggcataccccatggaatctggtacaaagcgtcagtcttgaagacaccatcacgtgtaggagactgattgtctgaccgtccagcaaaaagggcattataaatcttgctgttaaaggggtgaggggagatgcaggttgttcttttattcgccttgaactttttaattttcccggggttgcggagcgtgaacagttagcccgatctgatagcttgcaagattcaacagtttatccactacaggtcagagagatcgccgcagaagaaatgctcgtctcgtgttccagcacacatactggtgaagtcgttattttgccgaagggggggtaataaggttatgcaccccctctccacaccccagaatcattttttagctgggttcaaggcattagactttgcacatttttcccttaaacacccttgaaacgcggataaacagttgcatgtgcatcctaaaactaggtgagatgcgtactccgtgctccgataataacagtggtgttggggttgctgctagctcacgcactccgttcttttttttcaaccagcaaaattcgatggggagaaacttggggtactttgccgactcctccaccatgctggtatataaataatactcgcccacttttcgtttgctgcttttatatttcatagactgaaaaagactcttcttctactttttcataatatatctcagatatcactactatag

SEQ ID NO: 46 - DNA, 1020 bp, Pichia pastoris
cgcatttaaattgacttccttacaaaggggcttctgtttttgaggttccagttttctcataaactccaaccctgtagctctctctaatgcttctaatggtacttcaaaatctgtgagtttgacagaatttggtattggctcgtttggaaggacgaaagctgccagcgcaacatcaccagggtttcgtctattcttcgggtcctcggctacgaccaatttaaagaaatgcgtcggcactgcaactgatggcggacttccaatgagttcatatgttaccttccatttaccatcattaccatcctgcttaggcaaaaaaagaggacctgtaacaatgcgaactgatcgaaaatattgagttagagtacgagtaaagtactccaaatgagcccaataatctctgttaaaaccatctccaacttggggtgacatgttggtcaaaaaaaagtttcatccattgcgttttgagagaacttagcgtttgccgctggtgcttgatgccctctatcataaccagatcgaaaatagtcctttaatcttgccctaaatatgcttggaatttgctcatcttccttaaaaaaacaattctttctatcagcattgtgactggctaaagaatctggggtcaaatgttcaacgacataatatggattccgggtttgacggttgtatactgagacaaattctgctctggtttgtaaatcatggatgggaccaggaaaaccatacttgaaaaaatcagaaggtctcactattggagtctctagcgaaacagatgttgttggaggagataatgagctaggacttatggtagttggatttgcaactatagtgtcctttgccttactccaaaacattgatctggcaaaagctgagtatatagggaaagttactggtggaattgactaacctgcttagtttctggagcgcgctaaaacttcaattctttttccccgcgacaaaactttcaagtgtttgaaaccaaagctagcaccttcgaatagtcaaattagcgaattcgcg

SEQ ID NO: 47 - DNA, 508 bp, Pichia pastoris
gcgatttaaattcgcgaaagaacagcctaataaactccgaagcatgatggcctctatccggaaaacgttaagagatgtggcaacaggagggcacatagaatttttaaagacgctgaagaatgctatcatagtccgtaaaaatgtgatagtactttgtttagtgcgtacgccacttattcggggccaatagctaaacccaggtttgctggcagcaaattcaactgtagattgaatctctctaacaataatggtgttcaatcccctggctggtcacggggaggactatcttgcgtgatccgcttggaaaatgttgtgtatccctttctcaattgcggaaagcatctgctacttcccataggcaccagttacccaattgatatttccaaaaaagattaccatatgttcatctagaagtataaatacaagtggacattcaatgaatatttcattcaattagtcattgacactttcatcaacttactacgtcttattcaacaatgaattcgcg

SEQ ID NO: 48 - protein, 18 aa, Unknown
  Description of Unknown Signal sequence
MQVKSIVNLLLACSLAVA

SEQ ID NO: 49 - protein, 22 aa, Unknown
  Description of Unknown Signal sequence
MQFNWNIKTVASILSALTLAQA

SEQ ID NO: 50 - protein, 59 aa, Unknown
  Description of Unknown Signal sequence
MYRNLIIATALTCGAYSAYVPSEPWSTLTPDASLESALKDYSQTFGIAIKSLDADKIKR

SEQ ID NO: 51 - protein, 22 aa, Unknown
  Description of Unknown Signal sequence
MNLYLITLLFASLCSAITLPKR

SEQ ID NO: 52 - protein, 25 aa, Unknown
  Description of Unknown Signal sequence
MFEKSKFVVSFLLLLQLFCVLGVHG

SEQ ID NO: 53 - protein, 21 aa, Unknown
  Description of Unknown Signal sequence
MQFNSVVISQLLLTLASVSMG

SEQ ID NO: 54 - protein, 31 aa, Unknown
  Description of Unknown Signal sequence
MKSQLIFMALASLVASAPLEHQQQHHKHEKR

SEQ ID NO: 55 - protein, 19 aa, Unknown
  Description of Unknown Signal sequence
MKFAISTLLIILQAAAVFA

SEQ ID NO: 56 - protein, 23 aa, Unknown
  Description of Unknown Signal sequence
MKLLNFLLSFVTLFGLLSGSVFA

SEQ ID NO: 57 - protein, 20 aa, Unknown
  Description of Unknown Signal sequence
MIFNLKTLAAVAISISQVSA

SEQ ID NO: 58 - protein, 40 aa, Unknown
  Description of Unknown Signal sequence
MKISALTACAVTLAGLAIAAPAPKPEDCTTTVQKRHQHKR

SEQ ID NO: 59 - protein, 18 aa, Unknown
  Description of Unknown Signal sequence
MSYLKISALLSVLSVALA

SEQ ID NO: 60 - protein, 20 aa, Unknown
  Description of Unknown Signal sequence
MLSTILNIFILLLFIQASLQ

SEQ ID NO: 61 - protein, 36 aa, Unknown
  Description of Unknown Signal sequence
MKLSTNLILAIAAASAVVSAAPVAPAEEAANHLHKR

SEQ ID NO: 62 - protein, 19 aa, Unknown
  Description of Unknown Signal sequence
MFKSLCMLIGSCLLSSVLA

SEQ ID NO: 63 - protein, 19 aa, Unknown
  Description of Unknown Signal sequence
MKLAALSTIALTILPVALA

SEQ ID NO: 64 - protein, 23 aa, Unknown
  Description of Unknown Signal sequence
MSFSSNVPQLFLLLVLLTNIVSG

SEQ ID NO: 65 - protein, 46 aa, Unknown
  Description of Unknown Signal sequence
MQLQYLAVLCALLLNVQSKNVVDFSRFGDAKISPDDTDLESRERKR

SEQ ID NO: 66 - protein, 19 aa, Unknown
  Description of Unknown Signal sequence
MKIHSLLLWNLFFIPSILG

SEQ ID NO: 67 - protein, 19 aa, Unknown
  Description of Unknown Signal sequence
MSTLTLLAVLLSLQNSALA

SEQ ID NO: 68 - protein, 38 aa, Unknown
  Description of Unknown Signal sequence
MINLNSFLILTVTLLSPALALPKNVLEEQQAKDDLAKR

SEQ ID NO: 69 - protein, 16 aa, Unknown
  Description of Unknown Signal sequence
MFSLAVGALLLTQAFG

SEQ ID NO: 70 - protein, 16 aa, Unknown
  Description of Unknown Signal sequence
MKILSALLLLFTLAFA

SEQ ID NO: 71 - protein, 20 aa, Unknown
  Description of Unknown Signal sequence
MKVSTTKFLAVFLLVRLVCA

SEQ ID NO: 72 - protein, 19 aa, Unknown
  Description of Unknown Signal sequence
MQFGKVLFAISALAVTALG

SEQ ID NO: 73 - protein, 18 aa, Unknown
  Description of Unknown Signal sequence
MWSLFISGLLIFYPLVLG

SEQ ID NO: 74 - protein, 21 aa, Unknown
  Description of Unknown Signal sequence
MRNHLNDLVVLFLLLTVAAQA

SEQ ID NO: 75 - protein, 18 aa, Unknown
  Description of Unknown Signal sequence
MFLKSLLSFASILTLCKA

SEQ ID NO: 76 - protein, 20 aa, Unknown
  Description of Unknown Signal sequence
MFVFEPVLLAVLVASTCVTA

SEQ ID NO: 77 - protein, 22 aa, Unknown
  Description of Unknown Signal sequence
MVSLRSIFTSSILAAGLTRAHG

SEQ ID NO: 78 - protein, 22 aa, Unknown
  Description of Unknown Signal sequence
MFSPILSLEIILALATLQSVFA

SEQ ID NO: 79 - protein, 16 aa, Unknown
  Description of Unknown Signal sequence
MIINHLVLTALSIALA

SEQ ID NO: 80 - protein, 19 aa, Unknown
  Description of Unknown Signal sequence
MLALVRISTLLLLALTASA

SEQ ID NO: 81 - protein, 17 aa, Unknown
  Description of Unknown Signal sequence
MRPVLSLLLLLASSVLA

SEQ ID NO: 82 - protein, 24 aa, Unknown
  Description of Unknown Signal sequence
MVLIQNFLPLFAYTLFFNQRAALA

SEQ ID NO: 83 - protein, 21 aa, Unknown
  Description of Unknown Signal sequence
MKFPVPLLFLLQLFFIIATQG

SEQ ID NO: 84 - protein, 20 aa, Unknown
  Description of Unknown Signal sequence
MVSLTRLLITGIATALQVNA

SEQ ID NO: 85 - protein, 24 aa, Unknown
  Description of Unknown Signal sequence
MIFDGTTMSIAIGLLSTLGIGAEA

SEQ ID NO: 86 - protein, 31 aa, Unknown
  Description of Unknown Signal sequence
MVLVGLLTRLVPLVLLAGTVLLLVFVVLSGG

SEQ ID NO: 87 - protein, 16 aa, Unknown
  Description of Unknown Signal sequence
MLSILSALTLLGLSCA

SEQ ID NO: 88 - protein, 20 aa, Unknown
  Description of Unknown Signal sequence
MRLLHISLLSIISVLTKANA

SEQ ID NO: 89 - protein, 89 aa, Unknown
  Description of Unknown Signal sequence
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 90 - protein, 17 aa, Unknown
  Description of Unknown Signal sequence
MFKSVVYSILAASLANA

SEQ ID NO: 91 - protein, 19 aa, Unknown
  Description of Unknown Signal sequence
MLLQAFLFLLAGFAAKISA

SEQ ID NO: 92 - protein, 21 aa, Unknown
  Description of Unknown Signal sequence
MASSNLLSLALFLVLLTHANS

SEQ ID NO: 93 - protein, 29 aa, Unknown
  Description of Unknown Signal sequence
MNIFYIFLFLLSFVQGLEHTHRRGSLVKR

SEQ ID NO: 94 - protein, 48 aa, Unknown
  Description of Unknown Signal sequence
MLIIVLLFLATLANSLDCSGDVFFGYTRGDKTDVHKSQALTAVKNIKR

SEQ ID NO: 95 - protein, 49 aa, Unknown
  Description of Unknown Signal sequence
MESVSSLFNIFSTIMVNYKSLVLALLSVSNLKYARGMPTSERQQGLEER

SEQ ID NO: 96 - protein, 18 aa, Unknown
  Description of Unknown Signal sequence
MFAFYFLTACISLKGVFG

SEQ ID NO: 97 - protein, 23 aa, Unknown
  Description of Unknown Signal sequence
MRFSTTLATAATALFFTASQVSA

SEQ ID NO: 98 - protein, 23 aa, Unknown
  Description of Unknown Signal sequence
MKFAYSLLLPLAGVSASVINYKR

SEQ ID NO: 99 - protein, 22 aa, Unknown
  Description of Unknown Signal sequence
MKFFAIAALFAAAAVAQPLEDR

SEQ ID NO: 100 - protein, 15 aa, Unknown
  Description of Unknown Signal sequence
MQFFAVALFATSALA

SEQ ID NO: 101 - protein, 24 aa, Unknown
  Description of Unknown Signal sequence
MKWVTFISLLFLFSSAYSRGVFRR

SEQ ID NO: 102 - protein, 18 aa, Unknown
  Description of Unknown Signal sequence
MRSLLILVLCFLPLAALG

SEQ ID NO: 103 - protein, 15 aa, Unknown
  Description of Unknown Signal sequence
MKVLILACLVALALA

SEQ ID NO: 104 - protein, 18 aa, Unknown
  Description of Unknown Signal sequence
MFNLKTILISTLASIAVA

SEQ ID NO: 105 - protein, 20 aa, Unknown
  Description of Unknown Signal sequence
MYRKLAVISAFLATARAQSA

SEQ ID NO: 106 - protein, 85 aa, Saccharomyces cerevisiae
MRFPSIFTAVLFAASSALAAPVNTTTEDETAQIPAEAVIGYLDLEGDFDVAVLPFSNSTNNGLLFINTTIASIAAKEEGVQLDKR

SEQ ID NO: 107 - protein, 85 aa, Saccharomyces cerevisiae
MRFPPIFTAALFAASSALAAPANTTTEDETAQIPAEAVIGYLDSEGDSDVAVLPFSNSTNNGLSFINTTIASIAAKEEGVQLDKR

SEQ ID NO: 108 - protein, 85 aa, Saccharomyces cerevisiae
MRFPSIFTAVLFAASSALAAPANTTTEDETAQIPAEAVISYSDLEGDFDAAALPLSNSTNNGLSSTNTTIASIAAKEEGVQLDKR

SEQ ID NO: 109 - protein, 85 aa, Saccharomyces cerevisiae
MRPPSIFTAVLFAASSALAAPANTTTEDETTQIPAEAVATYLDLEGDVDVAVLPFSSSTNNGLSFINTTIASIAAKEEGVQLDKR

SEQ ID NO: 110 - protein, 85 aa, Saccharomyces cerevisiae
MRFPSIFTAALFAASSALAAPANTTTEGETAQTPAEAVIGYRDLEGDFDVAVLPFPNSTNNGLLFTNTTTASIAAKEEGVQLDKR

SEQ ID NO: 111 - protein, 85 aa, Saccharomyces cerevisiae
MRFPSIFTAVLLAAPSALAAPANATTEDEAAQIPAEAVIGYLDLEGDFDAAVLPFSNSTNNGLLSINTTIASIAAKEEGVQLDKR

SEQ ID NO: 112 - protein, 85 aa, Saccharomyces cerevisiae
MRFPSIFTAVVFAASSALAAPANTTAEDETAQIPAEAVIGYLGLEGDSDVAALPLSDSTNNGSLSTNTTIASIAAKEEGVQLDKR

SEQ ID NO: 113 - protein, 85 aa, Saccharomyces cerevisiae
MRLPSIFTAAVFAASSALAAPANTTTEDETAQIPAEAAIGYLDLEGDSDVAVLPLSNSTNNGLLFINTTIASIAAKEEGVQLDKR

SEQ ID NO: 114 - protein, 85 aa, Saccharomyces cerevisiae
MRFPSIFTAVLFAASSALAAPANTTTEDETAQIPAEAVIGYLDLEGDFDVAVLPFSNSTNDGLSFINTTTASIAAKEEGVQLDKR

SEQ ID NO: 115 - protein, 35 aa, Saccharomyces cerevisiae
MRFPSIFTAVLFAASSALAAPVNTTTEDETAQIPA

SEQ ID NO: 116 - protein, 37 aa, Pichia pastoris
MLSTILNIFILLLFIQASLQAPIPVVTKYVTEGIAVV

SEQ ID NO: 117 - protein, 36 aa, Pichia pastoris
MSFSSNVPQLFLLLVLLTNIVSGAVISVWSTSKVTK

SEQ ID NO: 118 - protein, 35 aa, Pichia pastoris
MNLYLITLLFASLCSAITLPKRDIIWDYSSEKIMG

SEQ ID NO: 119 - protein, 35 aa, Saccharomyces cerevisiae; identical to SEQ ID NO: 115

SEQ ID NO: 120 - protein, 35 aa, Pichia pastoris
MLSTILNIFILLLFIQASLQEFDYKDDDDKMVSKG

SEQ ID NO: 121 - protein, 35 aa, Unknown
  Description of Unknown D-EGFP sequence
MSFSSNVPQLFLLLVLLTNIVSGEFDYKDDDDKMV

SEQ ID NO: 122 - protein, 35 aa, Pichia pastoris
MNLYLITLLFASLCSAEFDYKDDDDKMVSKGEELF

SEQ ID NO: 123 - protein, 35 aa, Saccharomyces cerevisiae; identical to SEQ ID NO: 115

SEQ ID NO: 124 - protein, 35 aa, Pichia pastoris
MLSTILNIFILLLFIQASLQEFLPSGSDPAFSQPK

SEQ ID NO: 125 - protein, 35 aa, Unknown
  Description of Unknown D-CALB sequence
MSFSSNVPQLFLLLVLLTNIVSGEFLPSGSDPAFS

SEQ ID NO: 126 - protein, 35 aa, Pichia pastoris
MNLYLITLLFASLCSAEFLPSGSDPAFSQPKSVLD

SEQ ID NO: 127 - protein, 206 aa, Gallus gallus
MVAWWSLFLYGLQVAAPALAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 128 - protein, 243 aa, Gallus gallus
MRFPSIFTAVLFAASSALAAPVNTTTEDELEGDFDVAVLPFSASIAAKEEGVSLEKRAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 129 - protein, 205 aa, Gallus gallus
MRFPSIFTAVLFAASSALAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 130 - protein, 212 aa, Gallus gallus
MLGKNDPMCLVLVLLGLTALLGICQGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 131 - protein, 212 aa, Gallus gallus
MTKPTQVLVRSVSILFFITLLHLVVAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 132 - protein, 205 aa, Gallus gallus
MLLQAFLFLLAGFAAKISAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 133 - protein, 204 aa, Gallus gallus
MKWVTFISLLFLFSSAYSAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 134 - protein, 204 aa, Gallus gallus
MSFRSLLALSGLVCSGLAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 135 - protein, 202 aa, Gallus gallus
MKLAYSLLLPLAGVSAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 136 - protein, 247 aa, Gallus gallus
MRFPSIFTAVLFAASSALAAPVNTTTEDELEGDFDVAVLPFSASIAAKEEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 137 - protein, 24 aa, Gallus gallus
MAMAGVFVLFSFVLCGFLPDAAFG

SEQ ID NO: 138 - protein, 18 aa, Gallus gallus
MRSLLILVLCFLPLAALG

SEQ ID NO: 139 - protein, 89 aa, Gallus gallus
MRFPSIFTAVLFAASSALAAPVNTTTEDETAQIPAEAVIGYSDLEGDFDVAVLPFSNSTNNGLLFINTTIASIAAKEEGVSLDKREAEA

SEQ ID NO: 140 - protein, 19 aa, Gallus gallus
MKLILCTVLSLGIAAVCFA

SEQ ID NO: 141 - protein, 19 aa, Bos taurus
MKLFVPALLSLGALGLCLA

SEQ ID NO: 142 - protein, 19 aa, Sus scrofa
MKLFIPALLFLGTLGLCLA

SEQ ID NO: 143 - protein, 23 aa, Capra hircus
MESKALLLLALSVWLQSLTVSHG

SEQ ID NO: 144 - protein, 16 aa, Sus scrofa
MLLIWTLSLLLGAVLG

SEQ ID NO: 145 - protein, 674 aa, Gallus gallus
MYAAAAAAVAASPPRRDFISVTLSPEEAVGAGGYNNSKAWRRRSCWRKWKQLSRLQRSIILFLFAFLTVCGVISYTSVREPWKSLTSKSSDEHGTEPDAPGLRLANPAVLPAPQKADANAGDYPELSPQKPKLPHGRRNPSNFQIKPPWGDVRLQTRHDTRKAVEEPAQADKQEKTEKSVISWRGAVIEPDQSSEPPSSRVKEPEKPSSVEGESQKEPVPINERQMAVIEAFRHAWKGYKDFAWGHDELKPLSKSYSEWFGLGLTLIDALDTMWILGLREEFEEARKWVANDLAFDKNVDVNLFESTIRILGGLLSTYHLSGDSLFLEKAKDIGNRLMPAFKTPSKIPYSDVNIGRGTAHPPRWTSDSTVAEVTSIQLEFRELSRLTGDEKYQKAVDEVMKHVHTLSGKNDGLVPMFINTNSGQFTHLGVYTLGARADSYYEYLLKQWIQGGKTENELLEDYMKAIEGVKKHLLQRSQPKKLTFVGELAHGHFSAKMDHLVCFLPGTLALGAHNGLTADHMKLAEALIETCYQMYAQVETGLSPEIVHFNLHAQKGHKDVEIKPADRHNLLRPETVESLFYMYRFTGDKKYQDWGWEILQNFNKYTRVPTGGYTSINNVQNPSNPEPRDKMESFFLGETLKYMFLLFSDDIDLINLDKYVFNTEAHPLPIWVPA

SEQ ID NO: 146 - protein, 675 aa, Gallus gallus
MYAAAAAAVAASPPRRDFISVTLSPEEAVGAGGYNNSKAWRRRSCWRKWKQLSRLQRSIILFLFAFLTVCGVISYTSVREPWKSLTSKSSDEHGTEPDAPGLRLANPAVLPAPQKADANAGDYPELSPQKKPKLPHGRRNPSNFQIKPPWGDVRLQTRHDTRKAVEEPAQADKQEKTEKSVISWRGAVIEPDQSSEPPSSRVKEPEKPSSVEGESQKEPVPINERQMAVIEAFRHAWKGYKDFAWGHDELKPLSKSYSEWFGLGLTLIDALDTMWILGLREEFEEARKWVANDLAFDKNVDVNLFESTIRILGGLLSTYHLSGDSLFLEKAKDIGNRLMPAFKTPSKIPYSDVNIGRGTAHPPRWTSDSTVAEVTSIQLEFRELSRLTGDEKYQKAVDEVMKHVHTLSGKNDGLVPMFINTNSGQFTHLGVYTLGARADSYYEYLLKQWIQGGKTENELLEDYMKAIEGVKKHLLQRSQPKKLTFVGELAHGHFSAKMDHLVCFLPGTLALGAHNGLTADHMKLAEALIETCYQMYAQVETGLSPEIVHFNLHAQKGHKDVEIKPADRHNLLRPETVESLFYMYRFTGDKKYQDWGWEILQNFNKYTRVPTGGYTSINNVQNPSNPEPRDKMESFFLGETLKYMFLLFSDDIDLINLDKYVFNTEAHPLPIWVPA

SEQ ID NO: 147 - protein, 643 aa, Gallus gallus
MSAPALLPLAGRRLPALNLGASSFPHHRATLRLSEKFILLLILSAFITLCFGAFFFLPDSSKHKRFDLGLEDVLIPHVDTSKGGKHLGSFLIHGQGHDEHRHREEEERLRNKIRADHEKALEEAKEKLKKSRDEIQAEIQTEKNKVVQELKKKDSKPLPPVPLPNLVGINSGEPADPDIREKRNKIKEMMKHAWDNYRQYGWGHNELKPIARKGHSTNIFGNSQMGATIVDALDTLYIMGLRDEFREGQEWIDKNLDFSVNSEVSVFEVNIRFIGGLLAAYYLSGQEVFKIKAVQLAGKLLPAFNTPTGIPWAMVNLKSGVGRNWGWASAGSSILAEFGTLHMEFVHLSYLTGDPVYYNKVMHIRKLLQKMDRPNGLYPNYLNPRTGRWGQHHTSVGGLGDSFYEYLLKAWLMSDKTDTEARKMYDDAIEAIEKHLIRKSNGGLTFIGEWKNGHLERKMGHLTCFAGGMFALGADGSRDDKAGHYLQLGAEIAHTCHESYDRTTLKLGPEAFKFDGGVEAVAVRQNEKYYILRPEVIETYWYMWRFTHDPKYRQWGWEATQAIDKYCRVSGGFSGVKDVYSSSPTYDDVQQSFFLAETLKYLYLLFSNDDLLPLDNWVFNTEAHPLPVLHLANTTLSGNPAYR

SEQ ID NO: 148 - protein, 933 aa, Gallus gallus
MSGAAGCRGGGGERGPRWRRPWKLLALGLLSASSVLAAAPGAGAMSKEEKRRLGNQVLEMFDHAYSNYMDHAYPADELMPLTCRGRVRGQEPSRGDVDDALGKFSLTLIDTLDTLVVLNKTKEFEEAVKKVIKDVNLDNDIVVSVFETNIRVLGGLLGGHSVAIMLKDKGEYMQWYNGELLHMAKELGYKLLPAFNTTSGLPYPRVNLKFGVRHPEARTGTETDTCTACAGTLILEFAALSRFTGTSIFEEYARKALDFIWEKRQRSSNLVGVTINIHTGDWVRKDSGVGAGIDSYYEYLLKAYVLLGDDSFLERFNTHYDAIMKYISQPPLLLDVHIHKPMLNARTWMDSLLAFFPGLQVLKGDIRPAIETHEMLYQVIKKHNFLPEAFTTDFRVHWAQHPLRPEFAESTYFLYKATGDPYYLEVGKTLIENLNKYARVPCGFAAMKDVRTGSHEDRMDSFFLAEMFKYLYLLFADKEDMIFDIEDYIFTTEAHLLPLWLSTTNQTISKKNTTTEYTELDDSNFDWTCPNTQILFPNDPMFAQSIREPLKNVVDKSCPRSISRAEESLGTGPKPPLRARDFMASNPEHLEILKKMGVSLIHLKDGRVQLVQHAVQAASSLDAEDGLRFMQEMIELSSQQQKEQQLPPRAVQIVSHPFFGRVVLTAGPAQFGMDLSKHKSGTRGFVATIKPYNGCSEITNPEAVKEKIALMQRGQCMFAEKARNIQKAGAIGGIVIDDNEGSSSDTAPLFQMAGDGKNTDDITIPMLFLFNKEGNIILDAIREYEAVEVLLSDKAKDRDLEMENMDQKLSENDSHKQNSEEASSASQDVGAVSEEPEEGESSDVSDLDSLPPAQADTDSVSTSDQDSSIPGPGEAGAPEPACTQGDEQPQEQQTETESDSKVNWDNKVQPMESILADWNEDIEAFEMMEKDEL

SEQ ID NO: 149 - protein, 1007 aa, Felis catus
MGADARPLGVRAGGGGRGAARPGTSSRALPPPLPPLSFLLLLLAAPGARAAGYETCPMVHPDMLNVHLVAHTHDDVGWLKTVDQYFYGIHNDVQHAGVQYILDSVISSLLVEPTRRFIYVEIAFFSRWWHQQTNATQEVVRDLVRQGRLEFANGGWVMNDEAATHYGAIIDQMTLGLRFLEDTFGKDGRPRVAWHIDPFGHSREQASLFAQMGFDGLFFGRLDYQDKRVREENLGLEQVWRASASLKPPAADLFTSVLPNIYNPPEKLCWDTLCADKPFVEDRRSPEYNAEELVNYFLQLATAQGQHFRTNHTIMTMGSDFQYENANMWFRNLDRLIQLVNAQQQANGSRVNVLYSTPACYLWELNKANLTWSVKQDDFFPYADGPHQFWSGYFSSRPALKRYERLSYNFLQVCNQLEALAGPAANVGPYGSGDSAPLNQAMAVLQHHDAVSGTSKQHVADDYARQLAAGWDPCEVLLSNALARLSGSKEDFTYCRNLNVSVCPLSQTAKNFQVTIYNPLGRKIDWMVRLPVSKHGFVVRDPNGTVVPSDVVILPSSDGQELLFPASVPALGFSIYSVSQVPGQRPHAHKPQPRSQRPWSRVLAIQNEHIRARFDPDTGLLVEMENLDQNLLLPVRQAFYWYNASVGNNLSTQVSGAYIFRPNQEKPLMVSHWAQTRLVKTPLVQEVHQNFSAWCSQVVRLYRGQRHLELEWTVGPIPVGDGWGKEIISRFDTVLETKGLFYTDSNGREILERRRDYRPTWKLNQTETVAGNYYPVNSRIYIRDGNMQLTVLTDRSQGGSSLRDGSMELMVHRRLLKDDGRGVGEALLEDGLGRWVRGRHLVLLDKVRTAATGHRLQAEKEVLTPQVVLAPGGGAPYHLKVAPRKQFSGLRRELPPSVHLLTLARWDQKTLLLRLEHQFAVGEDSGNLSSPVTLDLTDLFSAFTITYLQETTLVANQLRASASRLKWTPNTGPTPLPSPSRLDPATITLQPMEIRTFLASVQWEEHG

SEQ ID NO: 150 - protein, 649 aa, Gallus gallus
MPAASLLPLFGSAAGPGALGGPAGGGAGGGGRKAAGPGAFRLTEKFVLLLVFSAFITLCFGAIFFLPDSSKLLSGVFFHSAALQPPPPPPGFQPRAPPQPGAGPAMPEEAGGAGSLERIRADHERALREAKETLQKLPEEIRRDIRQDKEKLLQDARGRKEAAAAGLPQRPFRQPVGAVGREPADLAVRQRRDKIKEMMKYAWDNYKRYAWGLNELKPISKQGHSSNLFGNIQGATIVDALDTLFIMEMKEEFKEAKEWVEKNLDFNVNAEISVFEVNIRFVGGLLSAYYLSGEEIFRKKAVELGEKLLPAFNTPTGIPWALLNIKSGIGRNWPWASGGSSILAEFGTLHLEFVHLSHLSGNPVFAEKVMNIRKVLSRLDKPEGLYPNYLNPSSGQWGQHHVSIGGLGDSFYEYLLKAWLMSDKTDEEGKKMYYDAVQAIETHLIRKSSGGLTYIAEWKGGLLEHKMGHLTCFAGGMFALGADGAPSDKTGHHIELGAEIARTCHESYDRTSMKLGPEAFRFDGGVEAIATRQNEKYYILRPEVIETYMYMWRLTHDPKYRQWAWEAVEALEKHCRVDGGYSGIRDVYSNHESHDDVQQSFFLSETLKYLYLLFSDDDLLPFEHWVFNTEAHPFPILRKEDGSKEEKEK

SEQ ID NO: 151 - protein, 893 aa, Trichoderma reesei; identical to SEQ ID NO: 7

SEQ ID NO: 152 - DNA, 1947 bp, Gallus gallus
atgccagctgcttctttgttgccattgtttggttctgctgctggtccaggtgctttgggtggtccagctggtggtggtgctggtggtggtggtagaaaggctgctggtccaggtgcttttagattgactgaaaagtttgttttgttgttggttttttctgcttttattactttgtgttttggtgctattttttttttgccagattcttctaagttgttgtctggtgttttttttcattctgctgctttgcaaccaccaccaccaccaccaggttttcaaccaagagctccaccacaaccaggtgctggtccagctatgccagaagaagctggtggtgctggttctttggaaagaattagagctgatcatgaaagagctttgagagaagctaaggaaactttgcaaaagttgccagaagaaattagaagagatattagacaagataaggaaaagttgttgcaagatgctagaggtagaaaggaagctgctgctgctggtttgccacaaagaccatttagacaaccagttggtgctgttggtagagaaccagctgatttggctgttagacaaagaagagataagattaaggaaatgatgaagtacgcttgggataactacaagagatacgcttggggtttgaacgaattgaagccaatttctaagcaaggtcattcttctaacttgtttggtaacattcaaggtgctactattgttgatgctttggatactttgtttattatggaaatgaaggaagaatttaaggaagctaaggaatgggttgaaaagaacttggattttaacgttaacgctgaaatttctgtttttgaagttaacattagatttgttggtggtttgttgtctgcttactacttgtctggtgaagaaatttttagaaagaaggctgttgaattgggtgaaaagttgttgccagcttttaacactccaactggtattccatgggctttgttgaacattaagtctggtattggtagaaactggccatgggcttctggtggttcttctattttggctgaatttggtactttgcatttggaatttgttcatttgtctcatttgtctggtaacccagtttttgctgaaaaggttatgaacattagaaaggttttgtctagattggataagccagaaggtttgtacccaaactacttgaacccatcttctggtcaatggggtcaacatcatgtttctattggtggtttgggtgattctttttacgaatacttgttgaaggcttggttgatgtctgataagactgatgaagaaggtaagaagatgtactacgatgctgttcaagctattgaaactcatttgattagaaagtcttctggtggtttgacttacattgctgaatggaagggtggtttgttggaacataagatgggtcatttgacttgttttgctggtggtatgtttgctttgggtgctgatggtgctccatctgataagactggtcatcatattgaattgggtgctgaaattgctagaacttgtcatgaatcttacgatagaacttctatgaagttgggtccagaagcttttagatttgatggtggtgttgaagctattgctactagacaaaacgaaaagtactacattttgagaccagaagttattgaaacttacatgtacatgtggagattgactcatgatccaaagtacagacaatgggcttgggaagctgttgaagctttggaaaagcattgtagagttgatggtggttactctggtattagagatgtttactctaaccatgaatctcatgatgatgttcaacaatctttttttttgtctgaaactttgaagtacttgtacttgttgttttctgatgatgatttgttgccatttgaacattgggtttttaacactgaagctcatccatttccaattttgagaaaggaagatggttctaaggaagaaaaggaaaag

SEQ ID NO: 153 - DNA, 1947 bp, Artificial Sequence
  Description of Artificial Sequence Synthetic polynucleotide
atgccagcagcatccttacttccattatttggctccgcagctggacctggcgctttaggtggtcctgctggcggcggagccggaggcggcggccgtaaagccgcaggtcctggtgcattcaggctgaccgagaaattcgtcctgctacttgtcttttcagcttttataacgctgtgtttcggcgcaattttttttcttcctgattcctccaaacttctttcaggtgtctttttccatagtgcagcacttcaacctcctcccccccctccaggtttccaacccagagctcctccacaaccaggagctggacctgccatgcccgaagaggcaggaggtgccggtagtctagaaagaataagggcagaccacgaaagagcacttcgtgaggctaaagaaaccctacagaaacttcccgaggagatccgtagggacataaggcaagataaagaaaaacttttacaagacgcacgtggtcgtaaagaagccgccgccgcaggactaccccaaagaccatttcgtcagcctgttggcgctgtcggaagggaacccgctgatcttgcagtaagacagagaagagacaaaatcaaggagatgatgaagtatgcctgggacaattataagcgttatgcctggggactaaatgagctaaaacctatttctaaacagggacacagttctaatttatttggaaacatccaaggtgccaccatagttgatgcacttgatactctgttcataatggagatgaaagaagagttcaaagaggcaaaagaatgggtagagaaaaaccttgatttcaacgtaaacgcagaaatcagtgtcttcgaagtaaatataagattcgttggaggcctactttccgcttattatttatcaggagaggaaatatttcgtaagaaggccgtggaattaggtgaaaaacttttgccagcttttaacaccccaacaggaattccttgggctttgttgaatatcaagagtggaatcggtagaaactggccttgggcttctggtggaagttcaatattggccgaatttggaactcttcatttagaattcgtccatttatcccatctaagtggtaacccagttttcgccgagaaagtaatgaatattcgtaaagttttgtctcgtcttgataagcctgagggcctgtaccctaactaccttaatccctcttcaggccaatggggccagcaccacgtgtccatcggcggtcttggagatagtttttatgagtatctgctgaaggcttggttaatgtccgacaagactgacgaagagggcaaaaagatgtattatgatgccgtccaagctatcgagactcacttaattaggaagtctagtggtggtctgacctatatagccgaatggaagggcggccttcttgaacacaaaatgggtcacttaacctgctttgcaggaggtatgtttgctttaggcgcagacggcgccccctcagataaaacgggacatcatattgagttaggagccgagattgccaggacatgccacgaatcatatgataggacgagtatgaagttaggtcctgaggcattcagatttgatggcggcgttgaggcaatcgctaccagacaaaatgagaaatactacattttaagaccagaagtcattgagacctacatgtacatgtggcgtctaactcatgaccccaaatatcgtcagtgggcatgggaggccgttgaagccctagaaaaacattgcagagttgacggcggttatagtggcatacgtgatgtctattcaaaccatgagtcccacgacgacgtacaacagtctttttttctttcagagacacttaagtacctatacctactattcagtgacgacgatcttctacctttcgaacattgggttttcaacaccgaagctcatcccttccccatcttacgtaaggaggacggttccaaagaggaaaaagagaaa

SEQ ID NO: 154 - protein, 272 aa, Homo sapiens
MRFPSIFTAVLFAASSALAAPVNTTTEDETAQIPAEAVIGYSDLEGDFDVAVLPFSNSTNNGLLFINTTIASIAAKEEGVSLDKREAEAQIPLCANLVPVPITNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 155 - protein, 4 aa, Unknown
  Description of Unknown ER retention signal sequence
HDEL

SEQ ID NO: 156 - protein, 4 aa, Unknown
  Description of Unknown ER retention signal sequence
KDEL